(19) United States
(12) Patent Application Publication
(10) Pub. No.: US 2025/0342959 A1
(43) Pub. Date: Nov. 6, 2025

(54) Prediction of Alzheimer's Disease

(71) Applicant: Bioscreening & Diagnostics LLC, Detroit, MI (US)

(72) Inventors: Ray Bahado-Singh, Grosse Pointe Shores, MI (US); Stewart F. Graham, Royal Oak, MI (US); Uppala Radhakrishna, Royal Oak, MI (US); Sangeetha Vishweswaraiah, Royal Oak, MI (US)

(73) Assignee: Bioscreening & Diagnostics LLC, Detroit, MI (US)

(21) Appl. No.: 18/866,362

(22) PCT Filed: May 16, 2023
(86) PCT No.: PCT/US2023/022401

§ 371 Date: Nov. 15, 2024

Related U.S. Application Data
(60) Provisional application No. 63/364,767, filed on May 16, 2022.

(51) Int. Cl. G16H 50/20; C12Q 1/6883; G06N 20/10; G16B 20/20; G16B 40/20; G16H 20/10; G16H 50/30; G16H 50/70

(52) U.S. Cl. CPC G16H 50/20; C12Q 1/6883; G06N 20/10; G16B 20/20; G16B 40/20; G16H 20/10; G16H 50/30; G16H 50/70; C12Q 2600/154

(57) ABSTRACT

A method for diagnosing Alzheimer's Disease or determining susceptibility to Alzheimer's Disease includes steps of obtaining a blood sample from a target subject and extracting cell-free (cf) DNA from the blood sample as extracted cf DNA. The degree of methylation in one or a plurality of Alzheimer indicator genes in the extracted cf DNA is identified. Each Alzheimer indicator gene identified is an indicator of the presence of or risk of developing Alzheimer's Disease where the plurality of Alzheimer indicators genes have been identified by a machine learning technique or by logistic regression. The target subject is identified as being at risk for Alzheimer's Disease if the amount of methylation of one or more Alzheimer's indicator genes differs from the amount of methylation established in control subjects not having Alzheimer's Disease to a statistically significant degree.

### CROSS-REFERENCE TO RELATED APPLICATIONS

This application claims the benefit of U.S. Provisional Patent Application 63/364,767, filed on May 16, 2022, which is hereby incorporated by reference in its entirety.

### TECHNICAL FIELD

In at least one aspect, the present invention is related to methods for diagnosing Alzheimer's Disease in a subject using circulating cell-free DNA.

### BACKGROUND

Late onset-Alzheimer's disease (AD) is the leading cause of severe dementia. The mechanism of the disease has not yet been resolved, however. The spectrum of AD patho-mechanisms is said to be wide and expanding (Hampel et al., 2018). Disease mechanistic information would yield very practical clinical benefits. For example, information on disease pathogenesis can set the stage for biomarker development and ultimately yield novel and druggable therapeutic targets. Given the long latency period and time course of AD, even in the absence of definitive treatment, therapies that slow disease progression or even reduce the amount of time spent in the severe dementia stages would reportedly significantly improve quality of life and yield substantial savings in healthcare costs (Winblad et al., 2016).

Epigenetic mechanisms regulate gene activity independent of DNA sequence changes (Handy et al., 2011) or mutations. DNA methylation is the most frequently studied epigenetic mechanism due to the wide availability of standardized laboratory techniques for its measurement (Kurdyukov and Bullock, 2016). DNA methylation changes are known to play a significant role in AD pathogenesis and offer the prospect of targeted correction given the current dearth of effective AD therapies (Esposito and Sherr, 2019).

There is intense research interest in the development of blood-based biomarkers for AD. The advantages include reduced reliance on invasive or expensive diagnostic techniques such as lumbar puncture, PET, and MRI imaging techniques (Hampel et al., 2019).

Circulating nucleic acid levels were found to be elevated in the plasma of AD patients, the plasma of a mouse model of AD, and in the culture medium of cells treated with amyloid-β (Pai et al., 2019) raising interest in using circulating nucleic acids as biomarkers for AD. Circulating cell-free DNA (cf DNA) is released from damaged, dead, and even living cells from different body tissues into the blood (Gai and Sun, 2019; Sun et al., 2015). Currently, circulating cf DNA, so-called ‘liquid biopsy’, is being used extensively in the study of cancer evolution. A major application has been the development of individualized drug therapies guided by patient-specific genetic and biological factors in cancer development (Hampel et al., 2019). There is significant interest in the application of cf DNA technologies in the study of AD. For example, neuronal, vascular, and inflammatory responses along with the anatomical and functional changes in the brain of AD cases could theoretically be monitored (Weinstein and Seshadri, 2014) given the fact that the DNA from cells from these different tissues contribute to the pool of circulating cf DNA.

Artificial Intelligence (AI) including Deep Learning (DL) offers distinct advantages in the analysis of the vast amount of biological data generated from ‘omics’ (including metabolomics and DNA-methylation) experiments (Alpay-Savasan et al., 2019; Bahado-Singh et al., 2018; Bahado-Singh et al., 2019b; Bahado-Singh et al., 2019d).

There is a need to develop new and more accurate methods for diagnosing Alzheimer's Disease.

### SUMMARY

In at least one aspect, a method for diagnosing Alzheimer's Disease or determining susceptibility to Alzheimer's Disease is provided. The method includes steps of obtaining a biological sample from a target subject and extracting cf DNA from the biological sample such as body fluid. The degree of methylation in one or a plurality of Alzheimer indicator genes (and more precisely epigenetically altered cytosine nucleotide aka CpG′ nucleotide(s) within these genes) from the extracted circulating cf DNA is identified. Each Alzheimer indicator gene identified is a marker of the presence of or risk of developing Alzheimer's Disease where the plurality of Alzheimer indicators genes have been identified by Artificial Intelligence (a machine learning technique) or by logistic regression. The target subject is identified as being at risk for Alzheimer's Disease if the amount of methylation of one or more Alzheimer indicator (CpG) genes differs from the amount of methylation established in control subjects not having Alzheimer's Disease by a predetermined amount or using a statistical threshold of significance.

In another aspect, a method for diagnosing Alzheimer's Disease or determining susceptibility to Alzheimer's Disease is provided. The method includes steps of obtaining a biological sample from a target subject and extracting circulating cf DNA from the biological sample. Gene methylation analysis is then performed on the extracted cf DNA to provide DNA methylation results. A trained neural network is applied to the gene methylation results to determine if the target subject is at increased risk for or has Alzheimer's disease, the trained neural network having been trained from genome-wide methylation training sets that include a first group of testing subjects having Alzheimer's disease and unaffected controls and a second independent group of the test (validation) subjects with and without Alzheimer's disease. The final objective is the development of a predictive algorithm that accurately identifies and distinguishes AD and unaffected cases.

In another aspect, methylation profiling of circulating cf DNA in AD cases and controls is performed.

In yet another aspect, pathway analysis is used to further understand the possible epigenetic and molecular mechanisms in AD where the pathway analysis is performed on the genes in the circulating cf DNA data.

In still another aspect, the accuracy of the epigenetic markers for AD prediction is evaluated.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIGS. 1A, 1B, 1C, 1D, 1E, and 1F show the detection of outliers in EPIC array methylation data. (A) Median signal intensity in sex chromosomes. (B) Median overall probe intensity. (C) Fraction of failed probes. Samples that deviate by more than 2 SD from the average fraction of failed probes are considered outliers. (D, E, and F) Principal component analysis.

FIGS. 2A, 2B, and 2C: Linear model of DNA methylation in association with cell-free circulating DNA in Alzheimer's disease: Robust linear models fitted to the DNA methylation data using Age, Sex, NeuN proportion, and Sentrix ID as covariates (A) Histogram based on p-value, showing CpGs with p-values less than 0.05, (B) Volcano plot showing CpGs with p-values less than 0.05 (orange colored nodes), (C) Overview of the methylation status of CpGs: Highest number of hyper-methylated CpGs (Green bar) were identified compared to hypo-methylated CpGs (Blue bar). The non-significant CpGs are presented using a grey scale.

FIG. 3 shows the visualization of Gene networks that have been epigenetically altered in AD and thus providing information on the molecular mechanisms of AD. The top 5 significant gene clusters (and significance levels) are depicted-Calcium signaling pathway (q=9.7×10⁻⁰⁵), Glutamatergic synapse (q=9.7×10⁻⁰⁵), Hedgehog signaling pathway (q=3.2×10⁻⁰⁴), Axon guidance (q=3.2×10⁻⁰⁴) and Olfactory transduction (q=4.4×10⁻⁰⁴).

FIG. 4 shows variance inflation analysis using all specified covariates (Full) and after the removal of inflated covariates (Reduced).

FIGS. 5A and 5B show the enrichment of genomic regions. (A) Enrichment of CpGs in various regions of the genome (CpG islands) and (B) the enrichment of genomic features including intergenic and within gene regions.

FIG. 6 shows the enrichment of differentially methylated genes in previously published neurological damage biomarkers gene panel. The correlation considered O'Connell et al., (2020) study with about 12,000 human subjects' mRNA expression data.

### DETAILED DESCRIPTION

Reference will now be made in detail to presently preferred compositions, embodiments, and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only to describe particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the invention can be obtained within a range of +/−5% of the indicated value.

The term “and/or” means that either all or only one of the elements of said group may be present.

It is also to be understood that this invention is not limited to the specific embodiments and methods described below, as specific components and/or conditions may, of course, vary. Furthermore, the terminology used herein is used only to describe particular embodiments of the present invention and is not intended to be limiting in any way.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5%, or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4, . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

The term “computing device” or “computer system” refers generally to any device or system that can perform at least one function, including communicating with another computing device or system for diagnosing AD. Sometimes the computing device is referred to as a computer.

When a computing device is described as performing an action or method step, it is understood that the computing devices are operable to perform the action or method step typically by executing one or more lines of source code. The actions or method steps can be encoded onto non-transitory memory (e.g., hard drives, optical drives, flash drives, and the like). In embodiments, the computing device has at least one processor and at least one memory, the memory comprising instructions executable by the processor to cause the processor to perform actions or stored in a data storage system.

Data storage system can include or be communicatively connected with one or more processor-accessible memories configured or otherwise adapted to store information for diagnosing AD. The memories can be, e.g., within a chassis or as parts of a distributed system. The phrase “processor-accessible memory” is intended to include any data storage device to or from which processor can transfer data (using appropriate components of peripheral system), whether volatile or nonvolatile; removable or fixed; electronic, magnetic, optical, chemical, mechanical, or otherwise. Exemplary processor-accessible memories include registers, floppy disks, hard disks, solid-state drives (SSDs), tapes, bar codes, Compact Discs, DVDs, read-only memories (ROM), erasable programmable read-only memories (EPROM, EEPROM, or Flash), and random-access memories (RAMs). The processor-accessible memories in the data storage system can be a tangible non-transitory computer-readable storage medium, i.e., a non-transitory device or article of manufacture that participates in storing instructions that can be provided to the processor for execution.

The processes, methods, or algorithms disclosed herein for diagnosing AD can be deliverable to or implemented by a computing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in a software executable object. Alternatively, the processes, methods, or algorithms can be embodied in whole or in part using suitable hardware components, such as Application Specific Integrated Circuits (ASICs), Field-Programmable Gate Arrays (FPGAs), state machines, controllers, or other hardware components or devices, or a combination of hardware, software and firmware components.

Machine learning (ML) teaches a machine how to perform a specific task and provide accurate results by identifying patterns. In embodiments, the computer device or computer system described herein is connected or includes a machine learning system for analyzing information for making a diagnosis of AD.

The term “subject” or “patient” refers to a human or other animals, including birds and fish as well as all mammals such as primates (particularly higher primates), horses, birds, fish sheep, dogs, rodents, guinea pigs, pig, cat, rabbits, and cows.

The term “biomarker” or “indicator (of a disease)” refers to any biological property, biochemical feature, or aspect that can be used to determine the presence or absence and/or the severity of a disease or disorder such as AD.

The term “cell-Free DNA (cf DNA)” refers to DNA that has been released from cells as a result of natural cell death/turnover etc or as a result of disease processes. The cf DNA is released into the circulation and rapidly broken down into DNA fragments and can ultimately end up in other body fluids. The techniques for the harvesting of cf DNA from the blood and other body fluids is well-known in the arts (Li Y et al. Size separation of circulatory DNA in maternal plasma permits ready detection of fetal DNA polymorphisms. Clin Chem 2004; 50:1002-1011; Zimmerman B et al. Noninvasive prenatal aneuploidy testing of chromosomes 13, 18, 21, X, and Y, using targeted sequencing of polymorphic loci. Prenat Diagn 2012; 32:1233-41).

The term “biological sample” refers to a sample from a subject. Examples of biological samples include tissue samples or body fluids. Examples of body fluids include blood, plasma, serum, urine, saliva, sputum, sweat, breath condensate, and tears.

Throughout this application, where publications are referenced, the disclosures of these publications in their entirety are hereby incorporated by reference into this application to more fully describe the state of the art to which this invention pertains.

### Abbreviations

“AD” means Alzheimer's Disease.
“AI” means artificial intelligence.
“cf DNA” or “CF DNA” means cell-free DNA.
“DL” means Deep Learning.
“FDR” means a false discovery rate.
“ML” means machine learning.
“SVM” means a support vector machine.
“GLM” means Generalized Linear Model (GLM).
“PAM” means Prediction Analysis for Microarrays.
“RF” means Random Forest (RF) and Linear Discriminant Analysis (LDA).

In embodiments, a method for diagnosing Alzheimer's Disease or determining susceptibility or risk to Alzheimer's Disease is provided. The method includes a step of obtaining a biological sample from a target subject, for example, a human, and extracting cf DNA from the biological sample, assaying the sample to determine the percentage of methylation of cytosine at loci throughout the genome; comparing the cytosine methylation level of the subject to control; and determining whether the subject has AD. The method can also include calculating the risk of the subject being diagnosed with AD based on the cytosine methylation level at multiple sites throughout the genome and integrating this information for accurate prediction. The control can be one or more characterized or known cases and/or a characterized or known group.

Examples of biological samples include body fluid, such as blood, plasma, serum, urine, saliva, sputum, sweat, breath condensate, and tears. The target subject can be an individual or a patient in need of (or in need thereof) diagnosis or experiencing symptoms of AD. The subject can also be undergoing routine screening for AD. Examples of target subjects include a human adult or an elderly human adult. In embodiments, the human adult is 50 years or older and the elderly human adult subject is 65 years or older.

The control subjects can be a well-characterized group of subjects or a population of normal (healthy) subjects. In embodiments, the control can be a well-characterized group of normal (healthy) people and/or a well-characterized population of AD patients.

Methylation Assays. Several quantitative methylation assays are available. These include COBRA™ which uses methylation-sensitive restriction endonuclease, gel electrophoresis, and detection based on labeled hybridization probes. Another available technique is the Methylation Specific PCR (MSP) for the amplification of DNA segments of interest. This is performed after sodium ‘bisulfite’ conversion of cytosine using methylation-sensitive probes. MethyLight™, a quantitative methylation assay-based, uses fluorescence-based PCR. Another method used is the Quantitative Methylation (QM™) assay, which combines PCR amplification with fluorescent probes designed to bind to putative methylation sites. Ms-SNuPET is a quantitative technique for determining differences in methylation levels in CpG sites. As with other techniques, bisulfite treatment is first performed leading to the conversion of unmethylated cytosine to uracil while methylcytosine is unaffected. PCR primers specific for bisulfite converted DNA are used to amplify the target sequence of interest. The amplified PCR product is isolated and used to quantitate the methylation status of the CpG site of interest. The preferred method of measurement of cytosine methylation is the Illumina method.

More comprehensive methylation information is provided by next-generation sequencing where DNA methylation information is provided at the of single cytosines throughout the entire genome. Sodium bisulfite conversion of the unmethylated cytosine to uracil which is then converted to thymine in a PCR reaction and then performing whole genome sequencing is performed. This is the gold standard for DNA methylation analysis and provides detailed information on gene regulation and transcription. Thus this approach may also be used in analyzing cytosine methylation in circulating cf DNA for AD detection. This technique is well-known in the arts.

Illumina Method. For DNA methylation assay the Illumina Infinium® Human Methylation 450 Beadchip or Illumina Infinium MethylationEPIC BeadChip assay can be used for quantitative methylation profiling. Briefly nucleic acid, for example, circulating cf DNA, is obtained. Using techniques widely known in the trade, the cf DNA is isolated using commercial kits. Proteins and other contaminants were removed from the cf DNA using proteinase K. The cf DNA is removed from the solution using available methods such as organic extraction, salting out, or binding the cf DNA to solid phase support.

Illumina's Infinium Human Methylation 450 Bead Chip system or Ilumina Infinium MethylationEPIC BeadCHip arrays can be used for genome-wide methylation analysis. Nucleic acid, such as circulating cf DNA, (500 ng) is subjected to bisulfite conversion to deaminate unmethylated cytosines to uracil with the EZ DNA Methylation Gold kit or EZ-96 Methylation Kit (Zymo Research) using the standard protocol for the Infinium assay. The cf DNA is enzymatically fragmented and hybridized to the Illumina BeadChips. BeadChips contain locus-specific oligomers and are in pairs, one specific for the methylated cytosine locus and the other for the unmethylated locus. A single base extension is performed to incorporate a biotin-labeled ddNTP. After fluorescent staining and washing, the BeadChip is scanned and the methylation status of each locus is determined using BeadStudio software (Illumina). Experimental quality was assessed using the Controls Dashboard that has sample-dependent and sample-independent controls for target removal, staining, hybridization, extension, bisulfite conversion, specificity, negative control, and non-polymorphic control. The methylation status is the ratio of the methylated probe signal relative to the sum of methylated and unmethylated probes. The resulting ratio indicates whether a locus is unmethylated (0) or fully methylated. Differentially methylated sites are determined using the Illumina Custom Model and filtered according to p value using 0.05 as a cutoff.

Bisulfite Conversion. As described in the Infinium® Assay Methylation Protocol Guide, nucleic acid, such as cf DNA, is treated with sodium bisulfite which converts unmethylated cytosine to uracil, while the methylated cytosine remains unchanged. The bisulfite converted cf DNA is then denatured and neutralized. The denatured cf DNA is then amplified. Bisulfite based analysis, the current technique for differentiating methylated from unmethylated cytosine, does not distinguish 5mC from 5hmC. New techniques include but are not limited to thin-layer chromatography assay, chemical tagging of 5hmC, immunoprecipitation, and commercially available 5hmC whole exome and even whole-genome sequencing techniques can be used to provide detailed information on epigenetic changes in cf DNA.

In embodiments, using the Illumina Infinium Assays for whole-genome (using genomic DNA) methylation studies, significant differences in the frequency (level or percentage) of methylation of specific cytosine nucleotides associated with particular CpGs within particular genes were demonstrated in the AD group when compared to a normal group. The differences in cytosine methylation levels are highly significant and of sufficient magnitude to accurately distinguish AD from the normal group. Thus, the methods described herein can be used to diagnose and screen for AD cases among a mixed population with AD and normal cases.

The whole-genome application process increases the amount of DNA by up to several thousand-fold. The next step uses enzymatic means to fragment the DNA. The fragmented DNA is next precipitated using isopropanol and separated by centrifugation. The separated DNA is next suspended in a hybridization buffer. The fragmented DNA is then hybridized to beads that have been covalently limited to 50mer nucleotide segments at a locus-specific to the cytosine nucleotide of interest in the genome. There is a total of over 500,000 bead types specifically designed to anneal to the locus where the particular cytosine is located. The beads are bound to silicon-based arrays. There are two bead types designed for each locus, one bead type represents a probe that is designed to match to the methylated locus at which the cytosine nucleotide will remain unchanged. The other bead type corresponds to an initially unmethylated cytosine which after bisulfite treatment is converted to a thiamine nucleotide. Unhybridized (not annealed to the beads) DNA is washed away leaving only DNA segments bound to the appropriate bead and containing the cytosine of interest. The bead-bound oligomer, after annealing to the corresponding patient DNA sequence, then undergoes single base extension with fluorescently-labeled nucleotide using the ‘overhang’ beyond the cytosine of interest in the patient DNA sequence as the template for extension.

If the cytosine of interest is unmethylated then it will match perfectly with the unmethylated or “U” bead probe. This enables single base extensions with fluorescent-labeled nucleotide probes and generates fluorescent signals for that bead probe that can be read in an automated fashion. If the cytosine is methylated, single base mismatch will occur with the “U” bead probe oligomer. No further nucleotide extension on the bead oligomer occurs however thus preventing the incorporation of the fluorescently tagged nucleotides on the bead. This will lead to a low fluorescent signal form the bead “U” bead. The reverse will happen on the “M” or methylated bead probe.

Laser is used to stimulate the fluorophore bound to the single base used for the sequence extension. The level of methylation at each cytosine locus is determined by the intensity of the fluorescence from the methylated compared to the unmethylated bead. Cytosine methylation level is expressed as “B” which is the ratio of the methylated bead probe signal to total signal intensity at that cytosine locus. These techniques for determining cytosine methylation have been previously described and are widely available for commercial use.

The present disclosure describes the use of a commercially available methylation technique to cover up to 99% Ref Seq genes involving close to 30,000 genes and 850,000 cytosine nucleotides down to the single nucleotide level, throughout the genome (Infinium MethylationEPIC BeadChip). The frequency of cytosine methylation at a single nucleotide level in a group of AD cases compared to controls is used to estimate the risk or probability of being diagnosed with AD. The cytosine nucleotides analyzed using this technique included cytosines within CpG islands and those at further distances outside of the CpG islands i.e. located in “CpG shores” and “CpG shelves” and even more distantly located from the island so-called “CpG seas”.

The cytosine evaluated as described herein includes but is not limited to cytosines in CpG islands located in the promoter regions of the genes. Other areas targeted and measured include the so-called CpG island ‘shores’ located up to 2000 base pairs distant from CpG islands and “shelves” which is the designation for DNA regions flanking shores. Even more distant areas from the CpG islands' so-called “seas” were analyzed for cytosine methylation differences. The extragenic cytosine loci, located outside of known genes (however they could potentially maintain long-distance control of unspecified genes) also detected AD with moderate, good, and excellent accuracy as indicated.

Identification of Specific Cytosine Nucleotides. Reliable identification of specific cytosine loci distributed throughout the genome has been detailed (Illumina) in the document: “CpG Loci Identification. A guide to Illumina's method for unambiguous CpG loci identification and tracking for the GoldenGate® and Infinium™ assays for Methylation.” A brief summary follows. Illumina has developed a unique CpG locus identifier that designates cytosine loci based on the actual or contextual sequence of nucleotides in which the cytosine is located. It uses a similar strategy as used by NCBI's re SNP IPS (rs #) and is based on the sequence flanking the cytosine of interest. Thus, a unique CpG locus cluster ID number is assigned to each of the cytosines undergoing evaluation. The system is reported to be consistent and will not be affected by changes in public databases and genome assemblies. Flanking sequences of 60 bases 5′ and 3′ to the CG locus (i.e. a total of 122 base sequences) are used to identify the locus. Thus, a unique “CpG cluster number” or cg # is assigned to the sequence of 122 bp which contains the CpG of interest. The cg # is based on Build 37 of the human genome (NCBI37). Accordingly, only if the 122 bp in the CpG cluster is identical is there a risk of a locus being assigned the same number and being located in more than one position in the genome. Three separate criteria are utilized to track individual CpG loci based on this unique ID system: chromosome number, genomic coordinate, and genome build. The lesser of the two coordinates “C” or “G” in CpG is used in the unique CG loci identification. The CG locus is also designated in relation to the first ‘unambiguous” pair of nucleotides containing either an ‘A’ (adenine) to ‘T’ (thiamine). If one of these nucleotides is 5′ to the CG then the arrangement is designated TOP and if such a nucleotide is 3′ it is designate BOT.

In addition, the forward or reverse DNA strand is indicated as being the location of the cytosine being evaluated. The assumption is made that the methylation status of cytosine bases within the specific chromosome region is synchronized.

As noted above Next Generation methylation sequencing is now considered the gold standard and can be used for and will even increase the precision and accuracy of AD detection using circulating cf DNA in patients being evaluated.

Cytosine Methylation for the diagnosing AD Using ROC Curve. To determine the accuracy of the methylation level of a particular cytosine locus for AD prediction, different threshold levels of methylation e.g. ≥5%, ≥10%, ≥20%, ≥30%, ≥40%, etc. at the site were used to calculate sensitivity and specificity for AD diagnosis or prediction of risk. Thus, for example, using ≥10% methylation at a particular cg locus, cases with methylation levels above this threshold would be considered to have a positive test, and those with lower than this threshold are interpreted as a negative methylation test. The percentage of AD cases with a positive test in this example, 10% methylation at this particular cytosine locus, would be equal to the sensitivity of the test. The percentage of normal (non-AD) cases with cytosine methylation levels of <10% at this locus would be considered the specificity of the test. False positive rate is here defined as the number of normal cases with a (falsely) abnormal test result and sensitivity is defined as the number of AD cases with (correctly) abnormal test result e.g. the level of methylation 10% at this particular CG location. A series of threshold methylation values are evaluated e.g. ≥5%, ≥1/10, ≥1/20, ≥1/30, etc., and used to generate a series of paired sensitivity and false positive values for each locus. A receiver operating characteristic (ROC) curve which is a plot of data points with sensitivity values on the Y-axis and false positivity rate on the X-axis is generated. This approach can be used to generate ROC curves for each individual cytosine locus that displays significant methylation differences between cases and AD groups. In this instance, the computer program ROCR package-version 3.4 (https://CRAN.R-project.org/package=ROCR) was used to generate the area under the ROC curves.

The ROC curve is a graph plotting sensitivity-defined in this setting as the percentage of AD cases with a positive test or abnormal cytosine methylation levels at a particular cytosine locus on the Y axis and false positive rate (1—specificity or 100%—specificity, when the latter is expressed as a percentage)—i.e. the number of normal (non-AD) cases with abnormal cytosine methylation at the same locus on the X-axis. Specificity is defined as the percentage of normal (non-AD) cases with normal methylation levels at the locus of interest or a negative test. False positive rate refers to the percentage of normal individuals falsely found to have a positive test (i.e. abnormal methylation levels); it can be calculated as 100—specificity (%) or expressed as a decimal format [1—specificity (expressed as a decimal point)].

The area under the ROC curves (AUC) indicates the accuracy of the test in identifying normal from abnormal cases. The AUC is the area under the ROC plot from the curve to the diagonal line from the point of intersection of the X- and Y-axes with an angle of incline of 45°. The higher the area under the ROC curve the greater the accuracy of the test in predicting the condition of interest. An area under the ROC=1.0 indicates a perfect test, which is positive (abnormal) in all cases with the disorder and negative in all normal cases (without the disorder). Methylation assay refers to an assay, many of which are commercially available, for determining the level of methylation at a particular cytosine in the genome. In this particular context, this approach can be used to distinguish the level of methylation in affected cases (AD) compared to unaffected controls.

Logistic regression analysis can be used for the calculation of sensitivity and specificity for the prediction of AD based on the methylation of cytosine loci.

Standard statistical testing using p-values to express the probability that the observed difference between cytosine methylation at a given locus between AD and control specimens can be performed. More stringent testing of statistical significance using the False Discovery Rate (FDR) for multiple comparisons was also performed. The FDR gives the probability that positive results were due to chance when multiple hypothesis testing is performed using multiple comparisons.

Statistical Analyses. The present disclosure describes a method for predicting, diagnosing, detecting AD in a subject, and/or calculating the risk of the subject being diagnosed with AD. One potential approach to this calculation can be based on logistic regression analysis leading to the identification of the significant independent predictors (e.g. clinical, demographic, etc) among a number of possible predictors (e.g. methylation loci) known to be associated with AD or increased risk of being diagnosed with AD. Cytosine methylation levels at different loci can be used by themselves or in combination with other known risk predictors for AD, such as prenatal exposure to toxins—“yes” or “no” (e.g. diabetes, age, gender combined with methylation levels in single or multiple loci) which are known to be associated with increased risk of AD as described in this application. For example, the probability of an individual being affected can be derived from the probability equation based on the logistic regression:

$${P}_{AD} = 1 / 1 + {e}^{- ( B 1 \times 1 + B 2 \times 2 + B 3 \times 3 … Bn \times n )}$$

where ‘x’ refers to the magnitude or quantity of the particular predictor (e.g. methylation level at a particular locus) and “β” or β-coefficient refers to the magnitude of change in the probability of the outcome (e.g., AD) for each unit change in the level of the particular predictor (x), the β values are derived from the results of the logistic regression analysis. These β values would be derived from multivariable logistic regression analysis in a large population of affected and unaffected individuals. Values for x1, x2, x3, etc., representing in this instance methylation percentage at different cytosine loci would be derived from the individual being tested while the β-values would be derived from the logistic regression analysis of the large reference population of affected (AD) and unaffected cases mentioned above. Based on these values, an individual's probability of having a type of AD can be quantitatively estimated. Probability thresholds are used to define individuals at high risk (e.g. a probability of ≥1/100 of AD may be used to define a high-risk individual triggering further evaluation involving memory impairment and cognitive ability, while individuals with risk <1/100 would require no further follow-up. Psychological testing is performed on individuals suspected of having AD. Numerous such tests exist. Among the most commonly used are the Mini-Mental State Exam (MMSE) and the Mini-Cog tests. The MMSE for example is composed of a series of questions that are designed to assess mental skills that are used in everyday functioning. designed The pathway for evaluation of patients for possible AD has been described by the National Institute of Aging and is summarized as follows. 1. Administer psychiatric evaluation to make sure that the symptoms are not due to depression or other mental health issues 2. Tests of memory, problem-solving, attention, counting, and language 3. Appropriate medical tests to rule out medical disorders that can explain symptoms and findings in the patient 4. Specialized tests such as CT scan, MRI, and Positron Emission tomography (PET) to support a diagnosis of AD. (Alzheimer's Disease and Related Dementias. National Institute of Aging). The threshold used will among other factors be based on the diagnostic sensitivity (number of AD cases correctly identified), specificity (number of non-AD cases correctly identified as normal), risk, and cost of related interventions pursuant to the designation of an individual as “high risk” for AD. Logistic regression analysis is well-known as a method in disease screening for estimating an individual's risk of having a disorder. (Royston P, Thompson S G. Model-based screening by risk with application in Down's syndrome. Stat Med 1992; 11:257-68.)

Individual risk of AD can also be calculated by using methylation percentages (reported as β-coefficients) at the individual discriminating cytosine locus by themselves or using different combinations of loci based on the method of overlapping Gaussian distribution or multivariate Gaussian distribution (Wald N J, Cuckle H S, Deusem J W, et al. (1988) Maternal serum screening for down syndrome in early pregnancy. BMJ 297, 883-887.) where the variable would be methylation level/percentage methylation at a particular (or multiple) loci so-called. Alternatively, if methylation percentages or β-coefficients are not normally distributed (i.e. non-Gaussian), normal Gaussian distribution would be achieved if necessary by the logarithmic transformation of these percentages.

As an example, two Gaussian distribution curves are derived for methylation at particular loci in the AD group and the normal populations. Mean, standard deviation and the degree of overlap between the two curves are then calculated. The ratio of the heights of the distribution curves at a given level of methylation will give the likelihood ratio or factor by which the risk of having AD is increased (or decreased) at a particular level of methylation at a given locus. The likelihood ratio (LR) value can be multiplied by the background risk of AD in the general population and thus give an individual's risk of AD based on methylation level at the CG site(s) chosen.

Each AD indicator CpG or biomarker is identified as being an indicator of the presence of or risk of developing AD. Characteristically, at least one or the plurality of AD indicator CpGs in multiple genes have been identified by a machine learning technique or by logistic regression. Finally, the target subject is identified as being at risk for Alzheimer's Disease if the amount of methylation of one or more Alzheimer's indicators genes differs from the amount of methylation established in control subjects (for the same genes) not having Alzheimer's Disease by a predetermined amount or using a statistical threshold of significance. In a refinement, the predetermined amount is at least a 30 percent difference in the amount of methylation as compared to control subjects (for corresponding genes between target subjects and controls). The percent different is ((|control−target subject|/control)*100%). In other refinements, the predetermined amount is at least, in increasing order of preference, 1 percent, 2, percent, 5 percent, 10 percent, 15 percent, 20 percent, 30 percent, 50 percent, 100 percent, or 200 percent difference in the amount of methylation as compared to control subjects (for corresponding genes between target subject and controls). It should be appreciated that ultimately, the predetermined amount is based on statistically significant differences in the amount of methylation as determined by statistical tests and/or statistical significance tests. In another refinement, the p-value is less than in increasing order of preference 0.05, 0.01, or 0.001 where the p-value is the probability of obtaining test results at least as extreme as the results actually observed during the test, assuming that the null hypothesis is correct.

Methylation refers to the enzymatic addition of a “methyl group” or single carbon atom to position #5 of the pyrimidine ring of cytosine which leads to the conversion of cytosine to 5-methyl-cytosine. The methylation of cytosine as described is accomplished by the actions of a family of enzymes named DNA methyltransferases (DNMTs). The κ-methyl-cytosine when formed is prone to mutation or the chemical transformation of the original cytosine to form thymine. Five-methyl-cytosines account for about 1% of the nucleotide bases overall in the normal genome. A gene can be hypermethylated or hypomethylated. Hypermethylation refers to increased frequency or percentage of methylation at a particular cytosine locus when specimens from an individual or group of interest are compared to a normal or control group. Hypomethylation refers to decreased frequency or percentage of methylation at a particular cytosine locus when specimens from an individual or group of interest are compared to a normal or control group.

The methylation of cytosines associated with or located in a gene is classically associated with the suppression of gene transcription. In some genes, however, increased methylation has the opposite effect and results in activation or increased transcription of a gene. One potential mechanism explaining the latter phenomenon is that methylation of cytosine could potentially inhibit the binding of gene suppressor elements thus releasing the gene from inhibition. Epigenetic modification, including DNA methylation, is the mechanism by which cells that contain identical DNA and genes experience the activation of different genes and result in the differentiation into unique tissues e.g. heart or intestines.

Artificial intelligence refers to the ability of computers to perform functions that were previously thought to require human intelligence. Aspects of AI include speech recognition and voice recognition. An advantage of AI is that it is able to segregate or classify groups e.g. AD cases as separate from controls based on the simultaneous use of a large number of discriminators e.g. CpG methylation level at multiple different CpG loci throughout the genome. The ability to simultaneously employ a large number of predictors e.g. 1000s or 100,000s significantly enhances the accuracy of detecting/predicting and discriminating disease cases from normal cases. AI is superior to conventional statistical techniques and logistic regression or human intelligence in these tasks. AI largely automates the process of generating a summary risk of AD based on the integration of data on DNA methylation across a large number of cytosines in the genome. As set forth above, a plurality of Alzheimer indicators CpGs have been identified using artificial intelligence (AI) including machine learning techniques or logistic regression. A particularly useful type of machine learning technique is a neural network method. Neural network refers to a machine learning model that can be trained with training input to approximate unknown functions. In a refinement, neural networks include a model of interconnected digital neurons that communicate and learn to approximate complex functions and generate outputs based on a plurality of inputs provided to the model. Additional examples of machine learning techniques that can be applied include but are not limited to support vector machine (SVM), a Generalized linear Model (GLM), Prediction Analysis for Microarrays (PAM), Random Forest (RF), and Linear Discriminant Analysis (LDA). Each of these approaches can be used to estimate AD risk. One or more AI algorithms, such as SVM, GLM, PAM, RF, LDA, and DL, can be used to improve the accuracy of predicting and/or diagnosing AD.

Deep Learning (DL): Deep-learning methods are representation-learning approaches with multiple levels of representation, obtained by composing simple but non-linear modules that each transform the representation at one level (starting with the raw input) into a representation at a higher, slightly more abstract level. With multiple such transformations, very complex functions can be learned. For classification tasks, higher layers of representation precisely target aspects of the input that are important for group discrimination while suppressing irrelevant variations. This type of hierarchical learning approach is particularly powerful as it allows the program to learn complex representations directly from the raw data. The approach is applicable to multiple disciplines.

Random Forest (RF): This is an increasingly utilized approach. RF generates many classifiers and aggregates their results. Common methods include boosting (Schapire and Yoram, 1998) and bagging (Breiman, 1996) of the classification trees. With boosting, successive trees give extra weight to points incorrectly predicted by earlier predictors. With bagging, successive trees do not depend on earlier trees—each is independently constructed using a bootstrap sample of the data set. RF adds an additional layer of randomness to bagging (Breiman, 2001). In addition to constructing each tree using a different bootstrap sample of the data, RF alters how the classification or regression trees are constructed. In standard trees, each node is split using the best split among all variables. In a random forest, each node is split using the best among a subset of predictors randomly chosen at that node. This approach performs very well compared to many other classifiers and is robust against overfitting (Breiman, 2001). In addition, it has only two parameters (the number of variables in the random subset at each node and the number of trees in the forest) and is generally not very sensitive to their values.

Support vector machine (SVM): SVMs (Cristianini and Shawe-Taylor, 2000) algorithms are relatively new. They display significant robustness even in the analysis of limited and noisy data. This has made them a platform of choice for varied applications from text categorization to bioinformatic analysis. SVMs are excellent classifiers and can separate a given set of binary labeled training data with a hyper-plane that is maximally distant from them (known as “the maximal margin hyper-plane”) (Boser et al., 1992). For situations in which linear separation of groups is not possible, SVMs can be combined with the technique of ‘kernels’ that automatically generates a non-linear mapping and separation to a feature space. The hyper-plane found by the SVM in the feature space corresponds to a non-linear decision boundary in the input space.

Linear Discriminant Analysis (LDA): Principal Component Analysis (PCA) and Linear Discriminant Analysis (LDA) are two commonly used techniques for data classification and dimensionality reduction. Linear Discriminant Analysis easily handles situations where the within-group frequencies are unequal, and their performances have been examined on randomly generated test data. LDA maximizes the ratio of between-class variance to the within-class variance in a data set thus guaranteeing maximal separation between groups (Balakrishnama and Ganapathiraju, 1998).

Prediction Analysis for Microarrays (PAM): is a statistical technique for class prediction using gene expression data using nearest shrunken centroids. The average gene expression level for each gene in each class is determined and divided by the within-class Standard Deviation. Thereafter the nearest shrunken centroid classification is calculated. This takes the gene expression profile of a new test group and compares it to each of the class centroids of the previously tested group. The class whose centroid it turns out to be the closest to is predicted to be the class of the new group. The nearest shrunken centroid refers to a further modification by which each of the class centroids is ‘shrunken’ to approach the values of the overall class centroid by a factor that is called the ‘threshold’ value. This is said to improve the accuracy of classification by minimizing the effect of less important contributing genes (Tibshirani et al., 2002). Thus class prediction is performed on a validation set. This method, therefore, identifies the subsets of genes that best characterizes and thus discriminates each class.

Generalized Linear Model (GLM): The generalized linear models (GLMs) are a broad class of models that include linear regression, ANOVA, Poisson regression, log-linear models, etc. But there are some limitations to GLM, such as linear function, which can have only a linear predictor in the systematic component, and responses must be independent.

In embodiments, an AI program executing on a computing device for calculating the risk of AD based on cf DNA methylation analysis executing at least part of the method is provided.

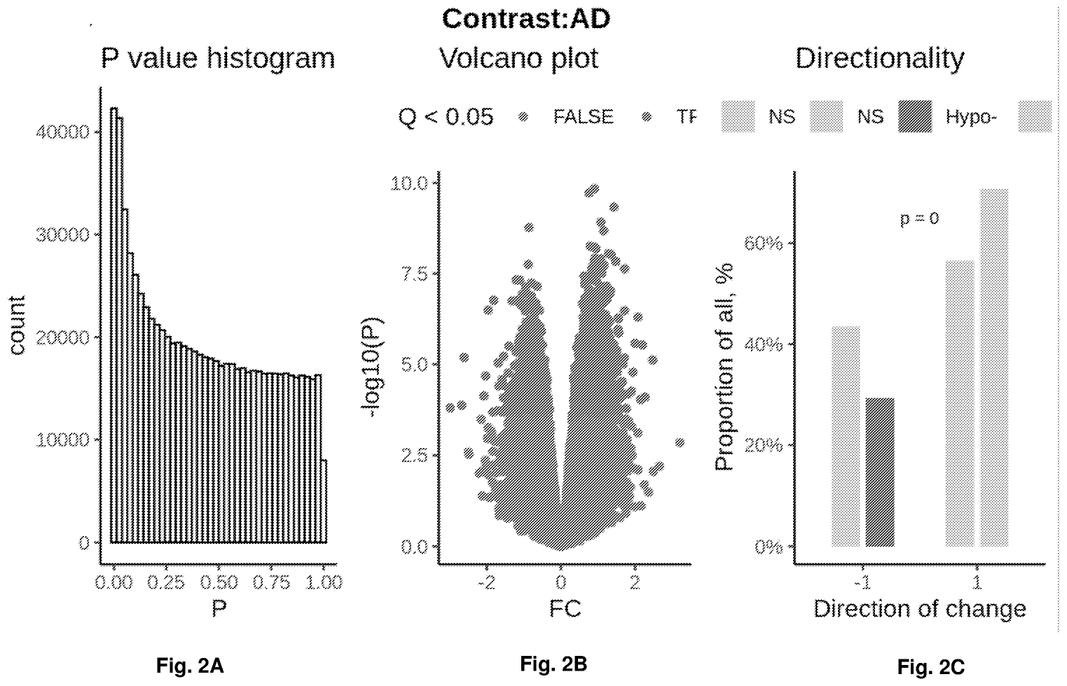

The present disclosure describes an abundance of cytosines with significantly altered methylation status. Based on the p-value histogram, a significant number of CpG methylation changes having a significance value less than 0.05 (FIG. 2A) was identified by the methods described herein, The number of CpG methylation changes is also reflected in the volcano plot (FIG. 2B). Overall, the methods described herein yielded a significantly higher number of hypermethylated CpGs (FIG. 2C). A statistically significant change in methylation (adjusted p<0.05) in a total of 3,684 CpGs was identified; among which 2,729 CpGs were found to be hypermethylated and the remaining 955 CpGs were hypomethylated in AD. 920 differentially methylated regions (DMRs) (adjusted p<0.05) were identified; among them, 854 DMRs were hypermethylated and the remaining 66 DMRs were hypomethylated.

Tables 1B, 2B, 3B, and 4B provide genomic loci that can be selected individually for use in the methods described herein to predict, detect, or diagnose AD in patients. One or more of Tables 1B, 2B, 3B, or 4B and one or more machine learning algorithms can be selected. One or more genomic loci from one of Tables 1B to 4B and one or more of the machine learning algorithms can be selected for predicting, detecting, or diagnosing AD in patients. In embodiments, one or more, two or more, three or more, four or more, up to and including all 100 of the genomic loci from one of Tables 1B to 4B (and one of the machine learning algorithms) can be selected. In embodiments, 5, 10, 15, 20, 25, 30, 35, 40, 45, 50, 55, 60, 65, 70, 75, 80, 85, 90, 95, or 100 genomic loci disclosed in Table 1B, 2B, 3B, or 4B (and one of the machine learning algorithms) can be selected to predict, detect, or diagnose AD in patients.

TABLE 1A
Results of cf-DNA AD-Intragenic (100 Variables
Cross-validation - Training Group)

| | SVM | GLM | PAM | RF | LDA | DL |
|---|---|---|---|---|---|---|
| AUC | 0.9810 | 0.9690 | 0.9890 | 0.9854 | 0.9493 | 0.9910 |
| 95% CI | (0.8800-1) | (0.8900-1) | (0.8900-1) | (0.8800-1) | (0.8800-1) | (0.9300-1) |
| SENSITIVITY | 0.9200 | 0.9200 | 0.9200 | 0.9200 | 0.9250 | 0.9350 |
| SPEC | 0.9220 | 0.9090 | 0.9080 | 0.9200 | 0.9250 | 0.9350 |

TABLE 1B
Results of cf-DNA AD-Intragenic (100 Variables
Cross-validation - Independent Test)

| | SVM | GLM | PAM | RF | LDA | DL |
|---|---|---|---|---|---|---|
| AUC | 0.9780 | 0.9683 | 0.9790 | 0.9755 | 0.9393 | 0.9890 |
| 95% CI | (0.8700-1) | (0.8800-1) | (0.8800-1) | (0.8800-1) | (0.8700-1) | (0.9250-1) |
| SENSITIVITY | 0.9100 | 0.9100 | 0.9100 | 0.9200 | 0.9250 | 0.9250 |
| SPEC | 0.9220 | 0.8990 | 0.8980 | 0.9100 | 0.9100 | 0.9350 |

SVM: cg14523095, cg10504568, cg08623971, cg16166011, cg07748806, cg04863005,
cg00360534, cg07018367, cg23313274, cg23736989, cg06183001, cg12647020, cg00249383,
cg02308140, cg24744710, cg06981876, cg12477067, cg14197110, cg16198754, cg07674600,
cg06288234, cg20227161, cg14209540, cg16667510, cg24621952, cg10287786, cg19681037,
cg07136344, cg15452937, cg06580014, cg02951237, cg07891658, cg15783299, cg13757935,
cg03585795, cg15721243, cg24268966, cg14016620, cg14488317, cg00182087, cg11101813,
cg14756780, cg10635347, cg27435943, cg23666682, cg04833918, cg18091083, cg05105770,
cg26019549, cg19290797, cg08500128, cg26952618, cg08429817, cg13286698, cg01317818,
cg04500050, cg27593649, cg05521175, cg07656025, cg27004481, cg18504632, cg13119036,
cg05147616, cg02374388, cg11658067, cg22888007, cg17898289, cg11646986, cg23283609,
cg15156528, cg25365217, cg20725500, cg00653017, cg11220060, cg24161613, cg13240253,
cg27421385, cg10640064, cg19781863, cg20987153, cg15186333, cg23145382, cg00151565,
cg07330481, cg01268901, cg05725404, cg13610910, cg01933778, cg10932166, cg02654372,
cg15448681, cg05981968, cg10349674, cg17006282, cg11625005, cg11169814, cg19731777,
cg12836863, cg12218359, cg07584910
cg19781863, cg20987153, cg15186333, cg23145382, cg00151565, cg07330481,
cg10640064, cg19781863, cg20987153, cg15186333, cg23145382, cg00151565, cg07330481,
cg01268901, cg05725404, cg13610910, cg01933778, cg10932166, cg02654372, cg15448681,
cg05981968, cg10349674, cg17006282, cg11625005, cg11169814, cg19731777, cg12836863,
cg12218359, cg07584910, cg19760734, cg05876416, cg00234736, cg21243612, cg24040188,
cg17674653, cg21942438, cg18322696, cg11748187, cg00266619, cg25645008, cg05210497,
cg04955826, cg14139646, cg19144827, cg19038282, cg20573828, cg23301353, cg21317441,
cg23962555, cg23576694, cg02749804, cg27304701, cg07188000, cg06601081, cg07295520,
cg25309859, cg05477521, cg06071033, cg07634627, cg19080490, cg21292587, cg22349396,
cg01321839, cg26176246, cg07604902, cg17307989, cg15399369, cg06080858, cg25592977,
cg15633396, cg03080505, cg04001333, cg20337969, cg04026948, cg00487979, cg23608903,
cg24818772, cg13672136, cg15512736
PAM: cg06183001, cg12647020, cg00249383, cg02308140, cg24744710, cg06981876,
cg12477067, cg14197110, cg16198754, cg07674600, cg14523095, cg10504568, cg08623971,
cg16166011, cg07748806, cg04863005, cg00360534, cg07018367, cg23313274, cg23736989,
cg06361127, cg11580390, cg06736683, cg06419732, cg07588934, cg05876950, cg10388349,
cg18149996, cg14544492, cg00637826, cg17359227, cg20074307, cg26807386, cg18546165,
cg01174459, cg26043567, cg07176064, cg10937807, cg27358947, cg21381949, cg18928066,
cg01779806, cg18105979, cg02214878, cg24736471, cg08484423, cg26174797, cg24582618,
cg27418687, cg23091723, cg26313511, cg07895657, cg14097631, cg01174708, cg22390660,
cg12724001, cg20642011, cg11146062, cg01821018, cg10593472, cg21694373, cg06198925,
cg22161147, cg21021332, cg21147040, cg15212455, cg19992375, cg23835821, cg11427310,
cg06235424, cg16549361, cg26160460, cg02734358, cg17190729, cg05962092, cg13722096,
cg18602114, cg16250093, cg27502912, cg25340983, cg03752609, cg06054410, cg15844438,
cg09535443, cg17375798, cg25902453, cg10087985, cg26799816, cg25958911, cg01626125,
cg26057559, cg09446760, cg17971695, cg18236571, cg24472965, cg20005649, cg22787826,
cg01978221, cg08189694, cg19419650
RF: cg24339519, cg14526576, cg02176715, cg09403277, cg12968558, cg04069932, cg17096965,
cg15135067, cg19086309, cg08558340, cg00651099, cg26975727, cg15275748, cg01385679,
cg18583094, cg02786267, cg11607339, cg10451247, cg03508346, cg04902126, cg13469814,
cg05289353, cg27269130, cg21402419, cg19397885, cg25411902, cg00782708, cg14161159,
cg11394247, cg10572670, cg07481154, cg27025857, cg14625772, cg04634182, cg00443946,
cg16897216, cg26401492, cg22551578, cg08514547, cg13982823, cg20040691, cg21695771,
cg00695458, cg23388763, cg04020590, cg18127680, cg11161318, cg24908186, cg01264438,
cg00625670, cg19285539, cg25068991, cg20955836, cg09738410, cg19411084, cg02747823,
cg09969919, cg16259171, cg02392667, cg22363621, cg01389234, cg16437904, cg05054124,
cg12723059, cg10922264, cg16445041, cg16519495, cg19643792, cg17034181, cg04845545,
cg00997754, cg17550299, cg07986378, cg04926736, cg05575436, cg00346623, cg25224620,
cg13447684, cg02861298, cg05781294, cg04070007, cg23300810, cg17412678, cg00343839,
cg23279578, cg15383187, cg04645130, cg00585187, cg05516004, cg19407331, cg10664053,
cg04752284, cg17514558, cg27085717, cg12798017, cg10886350, cg19645258, cg12648201,
cg23717186, cg11409367
cg02176715, cg09403277, cg12968558, cg04069932, cg17096965, cg15135067, cg19086309,
cg08558340, cg00651099, cg26975727, cg15275748, cg01385679, cg18583094, cg02786267,
cg11607339, cg10451247, cg03508346, cg04902126, cg13469814, cg05289353, cg27269130,
cg21402419, cg19397885, cg25411902, cg00782708, cg14161159, cg11394247, cg10572670,
cg07481154, cg27025857, cg14625772, cg04634182, cg00443946, cg16897216, cg26401492,
cg22551578, cg08514547, cg13982823
DL: cg19760734, cg05876416, cg00234736, cg21243612, cg24040188, cg17674653, cg21942438,
cg18322696, cg11748187, cg00266619, cg25645008, cg05210497, cg04955826, cg14139646,
cg19144827, cg19038282, cg20573828, cg23301353, cg21317441, cg23962555, cg23576694,
cg02749804, cg27304701, cg07188000, cg06601081, cg07295520, cg25309859, cg05477521,
cg06071033, cg07634627, cg19080490, cg21292587, cg22349396, cg01321839, cg26176246,
cg07604902, cg17307989, cg15399369, cg06080858, cg25592977, cg15633396, cg03080505,
cg04001333, cg20337969, cg04026948, cg00487979, cg23608903, cg24818772, cg13672136,
cg15512736, cg08432204, cg04238983, cg10421214, cg02083322, cg07572223, cg23659377,
cg12455465, cg17322500, cg27385729, cg26858144, cg08382737, cg21681168, cg20822767,
cg18461693, cg04184394, cg22661247, cg12795179, cg07738859, cg01894750, cg22174257,
cg02891314, cg23138872, cg11471498, cg16320684, cg01311909, cg00595051, cg22437221,
cg17040092, cg05856951, cg12647491, cg01638193, cg01916962, cg24489015, cg16579043,
cg17896683, cg11583863, cg20029201, cg14136101, cg19101624, cg20421983, cg14215483,
cg19714723, cg06773306, cg12255123, cg03551401, cg12000995, cg08259307, cg04895360,
cg09999719, cg04354845, cg14136101, cg19101624, cg20421983, cg14215483, cg19714723,
cg06773306, cg12255123, cg03551401, cg12000995, cg08259307, cg04895360, cg09999719,
cg04354845
GLM: cg08500128, cg26952618, cg08429817, cg13286698, cg01317818, cg04500050,
cg27593649, cg05521175, cg07656025, cg27004481, cg18504632, cg13119036, cg05147616,
cg02374388, cg11658067, cg22888007, cg17898289, cg11646986, cg23283609, cg15156528,
cg25365217, cg20725500, cg00653017, cg11220060, cg24161613, cg13240253, cg27421385,
1Q
LDA: cg15633396, cg03080505, cg04001333, cg20337969, cg04026948, cg00487979,
cg23608903, cg24818772, cg13672136, cg15512736, cg08432204, cg04238983, cg10421214,
cg02083322, cg07572223, cg23659377, cg12455465, cg17322500, cg27385729, cg26858144,
cg08382737, cg21681168, cg20822767, cg18461693, cg04184394, cg22661247, cg12795179,
cg07738859, cg01894750, cg22174257, cg02891314, cg23138872, cg11471498, cg16320684,
cg01311909, cg00595051, cg22437221, cg17040092, cg05856951, cg12647491, cg01638193,
cg01916962, cg24489015, cg16579043, cg17896683, cg11583863, cg20029201, cg14136101,
cg19101624, cg20421983, cg14215483, cg19714723, cg06773306, cg12255123, cg03551401,
cg12000995, cg08259307, cg04895360, cg09999719, cg04354845, cg24339519, cg14526576,

TABLE 2A
Results of 1-cf-DNA AD-Intragenic (100 Variables
Bootstrapping - Training Group)

| | SVM | GLM | PAM | RF | LDA | DL |
|---|---|---|---|---|---|---|
| AUC | 0.9890 | 0.9790 | 0.9890 | 0.9877 | 0.9524 | 1.0000 |
| 95% CI | (0.8700-1) | (0.8800-1) | (0.8800-1) | (0.8800-1) | (0.8700-1) | (0.9250-1) |
| SENSITIVITY | 0.9100 | 0.9200 | 0.9300 | 0.9200 | 0.9250 | 0.9350 |
| SPEC | 0.9120 | 0.8890 | 0.9180 | 0.9100 | 0.9200 | 0.9350 |

TABLE 2B
Results of cf-DNA AD-Intragenic (100 Variables
Bootstrapping - Independent Test Group)

| | SVM | GLM | PAM | RF | LDA | DL |
|---|---|---|---|---|---|---|
| AUC | 0.9810 | 0.9690 | 0.9810 | 0.9777 | 0.9424 | 0.9955 |
| 95% CI | (0.8700-1) | (0.8800-1) | (0.8800-1) | (0.8800-1) | (0.8700-1) | (0.9250-1) |
| SENSITIVITY | 0.9100 | 0.9100 | 0.9100 | 0.9200 | 0.9250 | 0.9250 |
| SPEC | 0.9220 | 0.8990 | 0.9080 | 0.9100 | 0.9200 | 0.9350 |

SVM: cg14523095, cg10504568, cg08623971, cg16166011, cg07748806, cg04863005,
cg00360534, cg07018367, cg23313274, cg23736989, cg06183001, cg12647020, cg00249383,
cg02308140, cg24744710, cg06981876, cg12477067, cg14197110, cg16198754, cg07674600,
cg06288234, cg20227161, cg14209540, cg16667510, cg24621952, cg10287786, cg19681037,
cg07136344, cg15452937, cg06580014, cg02951237, cg07891658, cg15783299, cg13757935,
cg03585795, cg15721243, cg24268966, cg14016620, cg14488317, cg00182087, cg11101813,
cg14756780, cg10635347, cg27435943, cg23666682, cg04833918, cg18091083, cg05105770,
cg26019549, cg19290797, cg08500128, cg26952618, cg08429817, cg13286698, cg01317818,
cg04500050, cg27593649, cg05521175, cg07656025, cg27004481, cg18504632, cg13119036,
cg05147616, cg02374388, cg11658067, cg22888007, cg17898289, cg11646986, cg23283609,
cg15156528, cg25365217, cg20725500, cg00653017, cg11220060, cg24161613, cg13240253,
cg27421385, cg10640064, cg19781863, cg20987153, cg15186333, cg23145382, cg00151565,
cg07330481, cg01268901, cg05725404, cg13610910, cg01933778, cg10932166, cg02654372,
cg15448681, cg05981968, cg10349674, cg17006282, cg11625005, cg11169814, cg19731777,
cg12836863, cg12218359, cg07584910
GLM: cg08500128, cg26952618, cg08429817, cg13286698, cg01317818, cg04500050,
cg27593649, cg05521175, cg07656025, cg27004481, cg18504632, cg13119036, cg05147616,
cg02374388, cg11658067, cg22888007, cg17898289, cg11646986, cg23283609, cg15156528,
cg25365217, cg20725500, cg00653017, cg11220060, cg24161613, cg13240253, cg27421385,
cg10640064, cg19781863, cg20987153, cg15186333, cg23145382, cg00151565, cg07330481,
cg01268901, cg05725404, cg13610910, cg01933778, cg10932166, cg02654372, cg15448681,
cg05981968, cg10349674, cg17006282, cg11625005, cg11169814, cg19731777, cg12836863,
cg12218359, cg07584910, cg19760734, cg05876416, cg00234736, cg21243612, cg24040188,
cg17674653, cg21942438, cg18322696, cg11748187, cg00266619, cg25645008, cg05210497,
cg04955826, cg14139646, cg19144827, cg19038282, cg20573828, cg23301353, cg21317441,
cg23962555, cg23576694, cg02749804, cg27304701, cg07188000, cg06601081, cg07295520,
cg25309859, cg05477521, cg06071033, cg07634627, cg19080490, cg21292587, cg22349396,
cg01321839, cg26176246, cg07604902, cg17307989, cg15399369, cg06080858, cg25592977,
cg15633396, cg03080505, cg04001333, cg20337969, cg04026948, cg00487979, cg23608903,
cg24818772, cg13672136, cg15512736
PAM: cg06183001, cg12647020, cg00249383, cg02308140, cg24744710, cg06981876,
cg12477067, cg14197110, cg16198754, cg07674600, cg14523095, cg10504568, cg08623971,
cg16166011, cg07748806, cg04863005, cg00360534, cg07018367, cg23313274, cg23736989,
cg06361127, cg11580390, cg06736683, cg06419732, cg07588934, cg05876950, cg10388349,
cg18149996, cg14544492, cg00637826, cg17359227, cg20074307, cg26807386, cg18546165,
cg01174459, cg26043567, cg07176064, cg10937807, cg27358947, cg21381949, cg18928066,
cg01779806, cg18105979, cg02214878, cg24736471, cg08484423, cg26174797, cg24582618,
cg27418687, cg23091723, cg26313511, cg07895657, cg14097631, cg01174708, cg22390660,
cg12724001, cg20642011, cg11146062, cg01821018, cg10593472, cg21694373, cg06198925,
cg22161147, cg21021332, cg21147040, cg15212455, cg19992375, cg23835821, cg11427310,
cg06235424, cg16549361, cg26160460, cg02734358, cg17190729, cg05962092, cg13722096,
cg18602114, cg16250093, cg27502912, cg25340983, cg03752609, cg06054410, cg15844438,
cg09535443, cg17375798, cg25902453, cg10087985, cg26799816, cg25958911, cg01626125,
cg26057559, cg09446760, cg17971695, cg18236571, cg24472965, cg20005649, cg22787826,
cg01978221, cg08189694, cg19419650
RF: cg24339519, cg14526576, cg02176715, cg09403277, cg12968558, cg04069932, cg17096965,
cg15135067, cg19086309, cg08558340, cg00651099, cg26975727, cg15275748, cg01385679,
cg18583094, cg02786267, cg11607339, cg10451247, cg03508346, cg04902126, cg13469814,
cg05289353, cg27269130, cg21402419, cg19397885, cg25411902, cg00782708, cg14161159,
cg11394247, cg10572670, cg07481154, cg27025857, cg14625772, cg04634182, cg00443946,
cg16897216, cg26401492, cg22551578, cg08514547, cg13982823, cg20040691, cg21695771,
cg00695458, cg23388763, cg04020590, cq18127680, cg11161318, cg24908186, cg01264438,
cg00625670, cg19285539, cg25068991, cg20955836, cg09738410, cg19411084, cg02747823,
cg09969919, cg16259171, cg02392667, cg22363621, cg01389234, cg16437904, cg05054124,
cg12723059, cg10922264, cg16445041, cg16519495, cg19643792, cg17034181, cg04845545,
cg00997754, cg17550299, cg07986378, cg04926736, cg05575436, cg00346623, cg25224620,
cg13447684, cg02861298, cg05781294, cg04070007, cg23300810, cg17412678, cg00343839,
cg23279578, cg15383187, cg04645130, cg00585187, cg05516004, cg19407331, cg10664053,
cg04752284, cg17514558, cg27085717, cg12798017, cg10886350, cg19645258, cg12648201,
cg23717186, cg11409367
LDA: cg15633396, cg03080505, cg04001333,
cg23608903, cg24818772, cg13672136, cg15512736, cg08432204, cg04238983, cg10421214,
cg02083322, cg07572223, cg23659377, cg12455465, cg17322500, cg27385729, cg26858144,
cg08382737, cg21681168, cg20822767, cg18461693, cg04184394, cg22661247, cg12795179,
cg07738859, cg01894750, cg22174257, cg02891314, cg23138872, cg11471498, cg16320684,
cg01311909, cg00595051, cg22437221, cg17040092, cg05856951, cg12647491, cg01638193,
cg01916962, cg24489015, cg16579043, cg17896683, cg11583863, cg20029201, cg14136101,
cg19101624, cg20421983, cg14215483, cg19714723, cg06773306, cg12255123, cg03551401,
cg12000995, cg08259307, cg04895360, cg09999719, cg04354845, cg24339519, cg14526576,
cg02176715, cg09403277, cg12968558, cg04069932, cg17096965, cg15135067, cg19086309,
cg08558340, cg00651099, cg26975727, cg15275748, cg01385679, cg18583094, cg02786267,
cg11607339, cg10451247, cg03508346, cg04902126, cg13469814, cg05289353, cg27269130,
cg21402419, cg19397885, cg25411902, cg00782708, cg14161159, cg11394247, cg10572670,
cg07481154, cg27025857, cg14625772, cg04634182, cg00443946, cg16897216, cg26401492,
cg22551578, cg08514547, cg13982823
DL: cg19760734, cg05876416, cg00234736, cg21243612, cg24040188, cg17674653, cg21942438,
cg18322696, cg11748187, cg00266619, cg25645008, cg05210497, cg04955826, cg14139646,
cg19144827, cg19038282, cg20573828, cg23301353, cg21317441, cg23962555, cg23576694,
cg02749804, cg27304701, cg07188000, cg06601081, cg07295520, cg25309859, cg05477521,
cg06071033, cg07634627, cg19080490, cg21292587, cg22349396, cg01321839, cg26176246,
cg07604902, cg17307989, cg15399369, cg06080858, cg25592977, cg15633396, cg03080505,
cg04001333, cg20337969, cg04026948, cg00487979, cg23608903, cg24818772, cg13672136,
cg15512736, cg08432204, cg04238983, cg10421214, cg02083322, cg07572223, cg23659377,
cg12455465, cg17322500, cg27385729, cg26858144, cg08382737, cg21681168, cg20822767,
cg18461693, cg04184394, cg22661247, cg12795179, cg07738859, cg01894750, cg22174257,
cg02891314, cg23138872, cg11471498, cg16320684, cg01311909, cg00595051, cg22437221,
cg17040092, cg05856951, cg12647491, cg01638193, cg01916962, cg24489015, cg16579043,
cg17896683, cg11583863, cg20029201, cg14136101, cg19101624, cg20421983, cg14215483,
cg19714723, cg06773306, cg12255123, cg03551401, cg12000995, cg08259307, cg04895360,
cg09999719, cg04354845

TABLE 3A
Results of 1-cf-DNA AD-Extragenic (100 Variables
Cross-validation - Training Group)

| | SVM | GLM | PAM | RF | LDA | DL |
|---|---|---|---|---|---|---|
| AUC | 0.9780 | 0.9610 | 0.9740 | 0.9880 | 0.9500 | 0.9933 |
| 95% CI | (0.8680-1) | (0.8776-1) | (0.8780-1) | (0.8866-1) | (0.8560-1) | (0.9120-1) |
| SENSITIVITY | 0.9200 | 0.9200 | 0.9200 | 0.9200 | 0.9250 | 0.9350 |
| SPEC | 0.9220 | 0.9090 | 0.9080 | 0.9200 | 0.9250 | 0.9350 |

Markers used are the same ones listed in Table 3 B

TABLE 3B
Results of cf-DNA AD-Extragenic (100 Variables
Cross-validation - Independent Test Group)

| | SVM | GLM | PAM | RF | LDA | DL |
|---|---|---|---|---|---|---|
| AUC | 0.9730 | 0.9455 | 0.9625 | 0.9610 | 0.9420 | 0.9899 |
| 95% CI | (0.8680-1) | (0.8776-1) | (0.8780-1) | (0.8866-1) | (0.8560-1) | (0.9120-1) |
| SENSITIVITY | 0.9100 | 0.9100 | 0.9100 | 0.9200 | 0.9250 | 0.9250 |
| SPEC | 0.9120 | 0.9090 | 0.9080 | 0.9100 | 0.9250 | 0.9350 |

Predictors in order:

cg16549063, cg17731069,
SVM: cg10163508, cg00631551, cg01699998, cg12308770, cg16549063, cg17731069,
cg00156330, cg07863545, cg10037749, cg13215579, cg22773231, cg03964954, cg18571488,
cg06070817, cg26026951, cg15572235, cg26373582, cg15979885, cg27614666, cg21828559,
cg18578690, cg11347946, cg04587141, cg02174133, cg20454464, cg12143028, cg04526584,
cg04196263, cg07030646, cg12081070, cg23330928, cg05031851, cg01799359, cg03073189,
cg16334555, cg03995102, cg12592387, cg11546554, cg01134758, cg18908062, cg10124079,
cg05089925, cg23948843, cg10678749, cg21776682, cg23901212, cg20932630, cg17379749,
cg14654363, cg08471498, cg04739153, cg13018639, cg24621754, cg14214257, cg06094776,
cg09547570, cg24400656, cg08781146, cg04071630, cg16557792, cg01969403, cg23680067,
cg20961509, cg20005578, cg13309071, cg23492823, cg02639223, cg19536605, cg07656520,
cg24650171, cg02756989, cg17626683, cg08679638, cg25432371, cg04938830, cg05506959,
cg08326079, cg25949806, cg12350164, cg08710469, cg26144909, cg25474687, cg09947625,
cg22759516, cg20786670, cg13605781, cg10067942, cg04747834, cg15773072, cg04871472,
cg15349886, cg24087404, cg16523364, cg01214923, cg10804656, cg04375046, cg14947623,
cg00442205, cg19062298, cg24561419
cg10809252, cg20604028, cg08628010, cg17864015, cg03668602, cg13708803, cg16703660,
cg16201634, cg21052905, cg12606317, cg23737109, cg24032030, cg21039341, cg11505731,
cg20355311, cg09590377, cg10228304, cg26044670, cg21583986, cg08200446, cg07195296,
cg21708703, cg16153919, cg07744798, cg12448977, cg18804499, cg01199628, cg25544413,
cg26570550, cg01680081, cg14449209, cg03625007, cg09368827, cg11296421, cg09596391,
cg08048268, cg07018435, cg07790752, cg10242172, cg02536698, cg21394171, cg09039561,
cg23491387, cg25801034, cg06585645, cg13557337, cg14454338, cg16236009, cg19395684,
cg03534031, cg13105425, cg15444358, cg11283860, cg15245556, cg10168494, cg22114896,
cg22509807, cg06055561, cg02179707, cg26074499, cg14089267, cg08576856, cg23001918,
cg01277599, cg15931375, cg17683100
RF: cg10168494, cg22114896, cg22509807, cg06055561, cg02179707, cg26074499, cg14089267,
cg08576856, cg23001918, cg01277599, cg15931375, cg17683100, cg16703660, cg16201634,
cg21052905, cg12606317, cg23737109, cg24032030, cg21039341, cg11505731, cg20355311,
cg09590377, cg10228304, cg26044670, cg21583986, cg08200446, cg07195296, cg21708703,
cg16153919, cg07744798, cg12448977, cg18804499, cg01199628, cg25544413, cg26570550,
cg01680081, cg14449209, cg03625007, cg09368827, cg11296421, cg09596391, cg08048268,
cg07018435, cg07790752, cg10242172, cg02536698, cg21394171, cg09039561, cg23491387,
cg25801034, cg06585645, cg13557337, cg14454338, cg16236009, cg19395684, cg03534031,
cg13105425, cg15444358, cg11283860, cg15245556, cg22521707, cg26237810, cg15153114,
cg23235671, cg24530489, cg18062092, cg17602206, cg02851625, cg15498294, cg11168104,
cg18340948, cg08451797, cg23951776, cg11188572, cg01256877, cg16045838, cg14294215,
cg01699762, cg21710377, cg06573787, cg15443223, cg22889444, cg03475293, cg02277646,
cg12893905, cg00460983, cg04597753, cg01796038, cg13171679, cg12271668, cg12485572,
cg06931676, cg15321570, cg21312057, cg02255986, cg04864378, cg15960490, cg16579144,
cg02739429, cg22790013
LDA: cg18340948, cg08451797, cg23951776, cg11188572, cg01256877, cg16045838,
cg14294215, cg01699762, cg21710377, cg06573787, cg15443223, cg22889444, cg03475293,
cg02277646, cg12893905, cg00460983, cg04597753, cg01796038, cg13171679, cg12271668,
cg12485572, cg06931676, cg15321570, cg21312057, cg02255986, cg04864378, cg15960490,
cg16579144, cg02739429, cg22790013, cg22521707, cg26237810, cg15153114, cg23235671,
cg24530489, cg18062092, cg17602206, cg02851625, cg15498294, cg11168104, cg21917512,
cg05232371, cg13565129, cg16271486, cg13160166, cg01640660, cg04897646, cg27127773,
cg27023252, cg24031760, cg16320141, cg16141338, cg07505327, cg08835755, cg16058196,
cg09145882, cg05624577, cg14701108, cg05785038, cg25178900, cg15079483, cg21279677,
cg24331722, cg14662218, cg14167603, cg00071446, cg02052531, cg01616085, cg07292773,
cg21155111, cg23609929, cg08657654, cg03431447, cg00019351, cg06310633, cg16232058,
cg13908477, cg06578342, cg24971112, cg12614325, cg07264726, cg24460235, cg01033191,
cg17174814, cg22417827, cg16153601, cg00813343, cg23829273, cg12695537, cg18774117,
cg02661473, cg05370462, cg03759229, cg05407003, cg07412315, cg19267910, cg11193213,
cg22265441, cg13529695, cg13423759
DL: cg00543415, cg12918536, cg19222397, cg17489635, cg13474332, cg19828063, cg18981569,
cg11737757, cg22534288, cg11826726, cg12945611, cg26102435, cg02160323, cg11861487,
cg13315609, cg10809252, cg16826168, cg20604028, cg05593139, cg08628010, cg24016690,
cg17864015, cg19341425, cg03668602, cg10367939, cg13708803, cg13666174, cg21136104,
cg12520929, cg17454247, cg24499764, cg07617678, cg04395970, cg16613631, cg03489427,
cg27102141, cg22045256, cg01780781, cg06203009, cg10843280, cg16703660, cg16201634,
cg21052905, cg12606317, cg23737109, cg24032030, cg21039341, cg11505731, cg20355311,
cg09590377, cg10228304, cg26044670, cg21583986, cg08200446, cg07195296, cg21708703,
cg16153919, cg07744798, cg12448977, cg18804499, cg01199628, cg25544413, cg26570550,
cg01680081, cg14449209, cg03625007, cg09368827, cg11296421, cg09596391,
cg07018435, cg07790752, cg10242172, cg02536698, cg21394171, cg09039561, cg23491387,
cg25801034, cg06585645, cg13557337, cg14454338, cg16236009, cg19395684, cg03534031,
cg13105425, cg15444358, cg11283860, cg15245556, cg10168494, cg22114896, cg22509807,
cg06055561, cg02179707, cg26074499, cg14089267, cg08576856, cg23001918, cg01277599,
cg15931375, cg17683100
GLM: cg15773072, cg04871472, cg15349886, cg24087404, cg16523364, cg01214923,
cg10804656, cg04375046, cg14947623, cg00442205, cg19062298, cg24561419, cg01969403,
cg23680067, cg20961509, cg20005578, cg13309071, cg23492823, cg02639223, cg19536605,
cg07656520, cg24650171, cg02756989, cg17626683, cg08679638, cg25432371, cg04938830,
cg05506959, cg08326079, cg25949806, cg12350164, cg08710469, cg26144909, cg25474687,
cg09947625, cg22759516, cg20786670, cg13605781, cg10067942, cg04747834, cg10124079,
cg05089925, cg23948843, cg10678749, cg21776682, cg23901212, cg20932630, cg17379749,
cg14654363, cg08471498, cg04739153, cg13018639, cg24621754, cg14214257, cg06094776,
cg09547570, cg24400656, cg08781146, cg04071630, cg16557792, cg02098816, cg07421597,
cg19508726, cg16661769, cg16058195, cg13667488, cg05442234, cg11169363, cg25468555,
cg09188096, cg04201021, cg26911448, cg18419576, cg08727218, cg10939445, cg18617411,
cg07535244, cg14395298, cg15368732, cg13666822, cg11829486, cg07184321, cg23122321,
cg16066205, cg08651677, cg04080417, cg19286744, cg27284586, cg19063162, cg23821954,
cg03785755, cg00953809, cg04604259, cg27298420, cg27609375, cg08711711, cg15782771,
cg04015057, cg11070274, cg19488431
PAM: cg12520929, cg17454247, cg24499764, cg07617678, cg04395970, cg16613631,
cg03489427, cg27102141, cg22045256, cg01780781, cg12945611, cg02160323, cg13315609,
cg16826168, cg05593139, cg24016690, cg19341425, cg10367939, cg13666174, cg21136104,
cg06203009, cg10843280, cg00543415, cg12918536, cg19222397, cg17489635, cg13474332,
cg19828063, cg18981569, cg11737757, cg22534288, cg11826726, cg26102435, cg11861487,

TABLE 4A
Results of 1-cf-DNA AD-Extragenic (100 Variables
Bootstrapping - Training Group)

| | SVM | GLM | PAM | RF | LDA | DL |
|---|---|---|---|---|---|---|
| AUC | 0.9920 | 0.9915 | 0.9977 | 0.9933 | 0.9677 | 1.0000 |
| 95% CI | (0.9000-1) | (0.9000-1) | (0.9000-1) | (0.9000-1) | (0.9500-1) | (0.9600-1) |
| SENSITIVITY | 0.9300 | 0.9300 | 0.9300 | 0.9300 | 0.9350 | 0.9550 |
| SPEC | 0.9420 | 0.9220 | 0.9280 | 0.9200 | 0.9350 | 0.9550 |

Markers used are the same ones listed in Table 4B

TABLE 4B
Results of cf-DNA AD-Extragenic (100 Variables
Bootstrapping - Independent Test Group)

| | SVM | GLM | PAM | RF | LDA | DL |
|---|---|---|---|---|---|---|
| AUC | 0.9870 | 0.9855 | 0.9925 | 0.9899 | 0.9599 | 0.9995 |
| 95% CI | (0.8900-1) | (0.8500-1) | (0.9000-1) | (0.9000-1) | (0.9500-1) | (0.9500-1) |
| SENSITIVITY | 0.9200 | 0.9200 | 0.9200 | 0.9300 | 0.9350 | 0.9450 |
| SPEC | 0.9320 | 0.9190 | 0.9180 | 0.9200 | 0.9350 | 0.9450 |

Predictors in order:

cg00631551,
SVM: cg10163508, cg00631551, cg01699998, cg12308770, cg16549063, cg17731069,
cg00156330, cg07863545, cg10037749, cg13215579, cg22773231, cg03964954, cg18571488,
cg06070817, cg26026951, cg15572235, cg26373582, cg15979885, cg27614666, cg21828559,
cg18578690, cg11347946, cg04587141, cg02174133, cg20454464, cg12143028, cg04526584,
cg04196263, cg07030646, cg12081070, cg23330928, cg05031851, cg01799359, cg03073189,
cg16334555, cg03995102, cg12592387, cg11546554, cg01134758, cg18908062, cg10124079,
cg05089925, cg23948843, cg10678749, cg21776682, cg23901212, cg20932630, cg17379749,
cg14654363, cg08471498, cg04739153, cg13018639, cg24621754, cg14214257, cg06094776,
cg09547570, cg24400656, cg08781146, cg04071630, cg16557792, cg01969403, cg23680067,
cg20961509, cg20005578, cg13309071, cg23492823, cg02639223, cg19536605, cg07656520,
cg24650171, cg02756989, cg17626683, cg08679638, cg25432371, cg04938830, cg05506959,
cg08326079, cg25949806, cg12350164, cg08710469, cg26144909, cg25474687, cg09947625,
cg22759516, cg20786670, cg13605781, cg10067942, cg04747834, cg15773072, cg04871472,
cg15349886, cg24087404, cg16523364, cg01214923, cg10804656, cg04375046, cg14947623,
cg00442205, cg19062298, cg24561419
cg09947625, cg22759516, cg20786670, cg13605781, cg10067942, cg04747834, cg10124079,
cg05089925, cg23948843, cg10678749, cg21776682, cg23901212, cg20932630, cg17379749,
cg14654363, cg08471498, cg04739153, cg13018639, cg24621754, cg14214257, cg06094776,
cg09547570, cg24400656, cg08781146, cg04071630, cg16557792, cg02098816, cg07421597,
cg19508726, cg16661769, cg16058195, cg13667488, cg05442234, cg11169363, cg25468555,
cg09188096, cg04201021, cg26911448, cg18419576, cg08727218, cg10939445, cg18617411,
cg07535244, cg14395298, cg15368732, cg13666822, cg11829486, cg07184321, cg23122321,
cg16066205, cg08651677, cg04080417, cg19286744, cg27284586, cg19063162, cg23821954,
cg03785755, cg00953809, cg04604259, cg27298420, cg27609375, cg08711711, cg15782771,
cg04015057, cg11070274, cg19488431
PAM: cg12520929, cg17454247, cg24499764, cg07617678, cg04395970, cg16613631,
cg03489427, cg27102141, cg22045256, cg01780781, cg12945611, cg02160323, cg13315609,
cg16826168, cg05593139, cg24016690, cg19341425, cg10367939, cg13666174, cg21136104,
cg06203009, cg10843280, cg00543415, cg12918536, cg19222397, cg17489635, cg13474332,
cg19828063, cg18981569, cg11737757, cg22534288, cg11826726, cg26102435, cg11861487,
cg10809252, cg20604028, cg08628010, cg17864015, cg03668602, cg13708803, cg16703660,
cg16201634, cg21052905, cg12606317, cg23737109, cg24032030, cg21039341, cg11505731,
cg20355311, cg09590377, cg10228304, cg26044670, cg21583986, cg08200446, cg07195296,
cg21708703, cg16153919, cg07744798, cg12448977, cg18804499, cg01199628, cg25544413,
cg26570550, cg01680081, cg14449209, cg03625007, cg09368827, cg11296421, cg09596391,
cg08048268, cg07018435, cg07790752, cg10242172, cg02536698, cg21394171, cg09039561,
cg23491387, cg25801034, cg06585645, cg13557337, cg14454338, cg16236009, cg19395684,
cg03534031, cg13105425, cg15444358, cg11283860, cg15245556, cg10168494, cg22114896,
cg22509807, cg06055561, cg02179707, cg26074499, cg14089267, cg08576856, cg23001918,
cg01277599, cg15931375, cg17683100
RF: cg10168494, cg22114896, cg22509807, cg06055561, cg02179707, cg26074499, cg14089267,
cg08576856, cg23001918, cg01277599, cg15931375, cg17683100, cg16703660, cg16201634,
cg21052905, cg12606317, cg23737109, cg24032030, cg21039341, cg11505731, cg20355311,
cg09590377, cg10228304, cg26044670, cg21583986, cg08200446, cg07195296, cg21708703,
cg16153919, cg07744798, cg12448977, cg18804499, cg01199628, cg25544413, cg26570550,
cg01680081, cg14449209, cg03625007, cg09368827, cg11296421, cg09596391, cg08048268,
cg07018435, cg07790752, cg10242172, cg02536698, cg21394171, cg09039561, cg23491387,
cg25801034, cg06585645, cg13557337, cg14454338, cg16236009, cg19395684, cg03534031,
cg13105425, cg15444358, cg11283860, cg15245556, cg22521707, cg26237810, cg15153114,
cg23235671, cg24530489, cg18062092, cg17602206, cg02851625, cg15498294, cg11168104,
cg18340948, cg08451797, cg23951776, cg11188572, cg01256877, cg16045838, cg14294215,
cg01699762, cg21710377, cg06573787, cg15443223, cg22889444, cg03475293, cg02277646,
cg12893905, cg00460983, cg04597753, cg01796038, cg13171679, cg12271668, cg12485572,
cg06931676, cg15321570, cg21312057, cg02255986, cg04864378, cg15960490, cg16579144,
cg02739429, cg22790013
cg24331722, cg14662218, cg14167603, cg00071446, cg02052531, cg01616085, cg07292773,
cg21155111, cg23609929, cg08657654, cg03431447, cg00019351, cg06310633, cg16232058,
cg13908477, cg06578342, cg24971112, cg12614325, cg07264726, cg24460235, cg01033191,
cg17174814, cg22417827, cg16153601, cg00813343, cg23829273, cg12695537, cg18774117,
cg02661473, cg05370462, cg03759229, cg05407003, cg07412315, cg19267910, cg11193213,
cg22265441, cg13529695, cg13423759
DL: cg00543415, cg12918536, cg19222397, cg17489635, cg13474332, cg19828063, cg18981569,
cg11737757, cg22534288, cg11826726, cg12945611, cg26102435, cg02160323, cg11861487,
cg13315609, cg10809252, cg16826168, cg20604028, cg05593139, cg08628010, cg24016690,
cg17864015, cg19341425, cg03668602, cg10367939, cg13708803, cg13666174, cg21136104,
cg12520929, cg17454247, cg24499764, cg07617678, cg04395970, cg16613631, cg03489427,
cg27102141, cg22045256, cg01780781, cg06203009, cg10843280, cg16703660, cg16201634,
cg21052905, cg12606317, cg23737109, cg24032030, cg21039341, cg11505731, cg20355311,
cg09590377, cg10228304, cg26044670, cg21583986, cg08200446, cg07195296, cg21708703,
cg16153919, cg07744798, cg12448977, cg18804499, cg01199628, cg25544413, cg26570550,
cg01680081, cg14449209, cg03625007, cg09368827, cg11296421, cg09596391, cg08048268,
cg07018435, cg07790752, cg10242172, cg02536698, cg21394171, cg09039561, cg23491387,
cg25801034, cg06585645, cg13557337, cg14454338, cg16236009, cg19395684, cg03534031,
cg13105425, cg15444358, cg11283860, cg15245556, cg10168494, cg22114896, cg22509807,
cg06055561, cg02179707, cg26074499, cg14089267, cg08576856, cg23001918, cg01277599,
GLM: cg15773072, cg04871472, cg15349886, cg24087404, cg16523364, cg01214923,
cg10804656, cg04375046, cg14947623, cg00442205, cg19062298, cg24561419, cg01969403,
cg23680067, cg20961509, cg20005578, cg13309071, cg23492823, cg02639223, cg19536605,
cg07656520, cg24650171, cg02756989, cg17626683, cg08679638, cg25432371, cg04938830,
cg05506959, cg08326079, cg25949806, cg12350164, cg08710469, cg26144909, cg25474687,
LDA: cg18340948, cg08451797, cg23951776, cg11188572, cg01256877, cg16045838,
cg14294215, cg01699762, cg21710377, cg06573787, cg15443223, cg22889444, cg03475293,
cg02277646, cg12893905, cg00460983, cg04597753, cg01796038, cg13171679, cg12271668,
cg12485572, cg06931676, cg15321570, cg21312057, cg02255986, cg04864378, cg15960490,
cg16579144, cg02739429, cg22790013, cg22521707, cg26237810, cg15153114, cg23235671,
cg24530489, cg18062092, cg17602206, cg02851625, cg15498294, cg11168104, cg21917512,
cg05232371, cg13565129, cg16271486, cg13160166, cg01640660, cg04897646, cg27127773,
cg27023252, cg24031760, cg16320141, cg16141338, cg07505327, cg08835755, cg16058196,
cg09145882, cg05624577, cg14701108, cg05785038, cg25178900, cg15079483, cg21279677,

For each of the AI platforms using intragenic CpG markers, there is extensive overlap between CpGs used in the different AI algorithms. The same applies to the extragenic CpGs. Table 5 (Intragenic markers and genes-consolidated list) is a consolidated list of all the separate intragenic CpGs (and associated genes) that have been used in the different AI algorithms. Similarly, Table 6 (Extragenic markers-consolidated list) lists all the independent extragenic CpG markers used in the 6 different AI algorithms for AD prediction and for which we are laying claims. Table 5 or 6 can be selected, and one or more genomic loci from one of Table 5 or 6 can be selected for predicting, detecting, or diagnosing AD in patients. In embodiments, one or more, two or more, three or more, four or more, up to and including all of the genomic loci from one of Table 5 or 6 can be selected. In embodiments, 5, 10, 15, 20, 25, 30, 35, 40, 45, 50, 55, 60, 65, 70, 75, 80, 85, 90, 95, or 100 genomic loci disclosed in Table 5 or 6 can be selected to predict, detect or diagnose AD in patients.

TABLE 5
Intragenic Markers and Genes Consolidated

| Cf DNA AD - Intragenic CpG | |
|---|---|
| markers (and genes) - Used in | |
| Cross-validation and | |
| Bootstrapping combined markers | Genes |
| cg14523095 | GCLC |
| cg16166011 | ADD2 |
| cg07748806 | ACTR3B |
| cg04863005 | TACSTD2 |
| cg00360534 | GALK2; MIR4716 |
| cg07018367 | RANBP17 |
| cg23313274 | TMEM98 |
| cg23736989 | RAD18 |
| cg06183001 | SIAH1 |
| cg12647020 | ARFGAP3 |
| cg00249383 | DNASE1L2 |
| cg02308140 | SNX10 |
| cg24744710 | KCNC2 |
| cg06981876 | MDFIC |
| cg12477067 | EIF5B |
| cg14197110 | CEP44 |
| cg16198754 | INO80 |
| cg07674600 | E2F7 |
| cg06288234 | TTC39A |
| cg20227161 | UCMA |
| cg14209540 | TMPO |
| cg16667510 | FNDC3A |
| cg24621952 | KIF3B |
| cg10287786 | DSCAML1 |
| cg19681037 | ENTPD1-AS1 |
| cg07136344 | GIT2 |
| cg15452937 | IPO11; LRRC70 |
| cg06580014 | C4orf52 |
| cg02951237 | NUFIP1 |
| cg07891658 | BANP |
| cg15783299 | MTUS1 |
| cg13757935 | MCTP1 |
| cg03585795 | ATP11B |
| cg15721243 | ZNF468 |
| cg24040188 | RBBP8 |
| cg17674653 | ARHGAP24 |
| cg21942438 | ZNF619 |
| cg18322696 | BNIP3L |
| cg11748187 | TCF7L2 |
| cg00266619 | FREM1 |
| cg25645008 | NEMP1 |
| cg05210497 | TIGD3 |
| cg04955826 | METAP1D |
| cg14139646 | OR5M8 |
| cg19144827 | ASCC3 |
| cg19038282 | DLG2 |
| cg20573828 | PARD3B |
| cg23301353 | SFMBT2 |
| cg21317441 | UBAC2; MIR548AN |
| cg23962555 | ANKRD12 |
| cg23576694 | DHX36 |
| cg02749804 | CASC3 |
| cg27304701 | LOC101929584 |
| cg07188000 | SRD5A3 |
| cg06601081 | HTT |
| cg07295520 | FRS2 |
| cg25309859 | CMPK1 |
| cg05477521 | YPEL2 |
| cg06071033 | HCN4 |
| cg07634627 | MECR |
| cg19080490 | SNAP23 |
| cg21292587 | FOXP2 |
| cg22349396 | CHRNE; C17orf107 |
| cg01321839 | ICOS |
| cg26176246 | SLC34A2 |
| cg07604902 | PBX1 |
| cg17307989 | RGS6 |
| cg17190729 | LIMK2 |
| cg05962092 | KCNA7 |
| cg13722096 | LINC00689 |
| cg18602114 | C10orf116 |
| cg16250093 | MGRN1 |
| cg27502912 | CPT1B; CHKB-CPT1B |
| cg25340983 | TBCD |
| cg03752609 | MLLT10 |
| cg06054410 | SLC44A1 |
| cg15844438 | MAML3 |
| cg09535443 | LOC283140 |
| cg17375798 | NMI |
| cg25902453 | LINC01047; LINC00440 |
| cg10087985 | PAQR9-AS1 |
| cg26799816 | ADAM23 |
| cg25958911 | BOP1 |
| cg01626125 | ZNF84 |
| cg26057559 | MKLN1 |
| cg09446760 | IPO8 |
| cg17971695 | FAM178B |
| cg18236571 | PABPC4L |
| cg24472965 | NXPH2 |
| cg20005649 | LOC285766 |
| cg22787826 | SIM1 |
| cg01978221 | CTSL |
| cg08189694 | LOC100507424 |
| cg19419650 | EVI5L |
| cg24339519 | KLHL24 |
| cg14526576 | HIPK2 |
| cg02176715 | OR3A2 |
| cg09403277 | WDR60 |
| cg12968558 | C15orf41 |
| cg04069932 | NR4A1 |
| cg05575436 | DOPEY1 |
| cg00346623 | BTBD9 |
| cg25224620 | IPO5 |
| cg13447684 | MAD1L1 |
| cg02861298 | AKAP2; PALM2-AKAP2 |
| cg05781294 | BAT1; SNORD117 |
| cg04070007 | CELF2 |
| cg23300810 | HBS1L |
| cg17412678 | LARS2 |
| cg00343839 | LOC728392 |
| cg23279578 | TMEM43 |
| cg15383187 | MORC2 |
| cg04645130 | RAB31 |
| cg00585187 | CTDSPL |
| cg05516004 | ADARB1 |
| cg19407331 | LOC100505716; BRE |
| cg10664053 | ZNF148 |
| cg04752284 | FAM207A |
| cg17514558 | PCDHB19P |
| cg27085717 | KMT2A |
| cg12798017 | SGMS2; LOC101929595 |
| cg10886350 | SMARCA2 |
| cg19645258 | AKAP13 |
| cg10504568 | DENND1A |
| cg24268966 | HMGA2 |
| cg14016620 | GNAQ |
| cg14488317 | OSBPL5 |
| cg00182087 | DUXA |
| cg11101813 | RPRD1A |
| cg14756780 | MYH11 |
| cg10635347 | TM2D3 |
| cg27435943 | ADK; LOC102723439 |
| cg23666682 | COPS7B |
| cg04833918 | PRKD3 |
| cg18091083 | RPTOR |
| cg05105770 | HIF1A |
| cg26019549 | TNFRSF11B |
| cg19290797 | BRE |
| cg08500128 | MAGOHB |
| cg26952618 | FAM18A |
| cg08429817 | SLC35B4 |
| cg13286698 | TDH |
| cg01317818 | HACD4 |
| cg04500050 | TRPS1 |
| cg27593649 | SLC29A1 |
| cg05521175 | OBSL1 |
| cg07656025 | FAM89A |
| cg27004481 | PDLIM5 |
| cg18504632 | EIF4E3 |
| cg13119036 | BRE |
| cg05147616 | SCAPER |
| cg02374388 | JPH2 |
| cg11658067 | GAPVD1 |
| cg22888007 | FNIP2 |
| cg17898289 | CEP152 |
| cg11646986 | PID1 |
| cg23283609 | USP32 |
| cg15156528 | TUBA 1A |
| cg15399369 | LOC101927292 |
| cg06080858 | OSBP2 |
| cg25592977 | CA10 |
| cg15633396 | AZIN1-AS1 |
| cg03080505 | FBXO36 |
| cg04001333 | FLVCR2 |
| cg20337969 | WDR77 |
| cg04026948 | PGCP |
| cg00487979 | MAML3 |
| cg23608903 | TM2D1 |
| cg24818772 | GSTO2 |
| cg13672136 | LTF |
| cg15512736 | GLIPR1 |
| cg06361127 | ZNF648 |
| cg11580390 | SRP14 |
| cg06736683 | SLC4A5 |
| cg06419732 | CDK18 |
| cg07588934 | FGFR1OP2 |
| cg05876950 | ARL6 |
| cg10388349 | ZSWIM6 |
| cg18149996 | EDEM2 |
| cg14544492 | LOC339529 |
| cg00637826 | DUSP16 |
| cg17359227 | WAPAL |
| cg20074307 | SAMD4A |
| cg26807386 | CLMN |
| cg18546165 | CMKLR1 |
| cg01174459 | C12orf75 |
| cg26043567 | SNF8 |
| cg07176064 | HMGXB4 |
| cg10937807 | DIP2C |
| cg27358947 | ENTPD1; ENTPD1-AS1 |
| cg21381949 | LEPREL1 |
| cg18928066 | BTBD19 |
| cg17096965 | PSME3 |
| cg15135067 | MAP3K7CL |
| cg19086309 | CALD1 |
| cg08558340 | SRRT |
| cg00651099 | ANKRD50 |
| cg26975727 | DNAJC5B |
| cg15275748 | CFAP70 |
| cg01385679 | ELMO2 |
| cg18583094 | C11orf63 |
| cg02786267 | HLA-DQA2 |
| cg11607339 | ZNF407 |
| cg10451247 | DLG2 |
| cg03508346 | NOX4 |
| cg04902126 | SLC39A10 |
| cg13469814 | HTR7 |
| cg05289353 | MON2 |
| cg27269130 | CDC42EP5 |
| cg21402419 | PCCA |
| cg19397885 | VWDE |
| cg25411902 | ISM1 |
| cg00782708 | C2orf34 |
| cg14161159 | C2orf27A |
| cg11394247 | HEATR5B |
| cg10572670 | RGNEF |
| cg07481154 | LPP |
| cg27025857 | LOC400655 |
| cg14625772 | CCDC59; METTL25 |
| cg04634182 | ZBTB47 |
| cg00443946 | LOC732275 |
| cg16897216 | HSD11B1 |
| cg26401492 | SFMBT1 |
| cg22551578 | BLCAP; NNAT |
| cg08514547 | SGMS1-AS1 |
| cg13982823 | HMGB1 |
| cg23717186 | ITPKB |
| cg11409367 | ACTL6A |
| cg08432204 | NCOA7 |
| cg04238983 | MIR612 |
| cg10421214 | BCKDHB |
| cg02083322 | MTHFD1L |
| cg07572223 | TTC18 |
| cg23659377 | GABARAPL1 |
| cg12455465 | OR4F15 |
| cg17322500 | ZNF44 |
| cg27385729 | DDX6 |
| cg26858144 | CACNG8 |
| cg08382737 | LIN7B |
| cg21681168 | CCK |
| cg20822767 | CYP20A1 |
| cg18461693 | NSF |
| cg04184394 | MCF2L2 |
| cg22661247 | PILRA |
| cg12795179 | FKTN |
| cg07738859 | MAD1L1 |
| cg01894750 | CEP57 |
| cg22174257 | IL27 |
| cg02891314 | GFPT2 |
| cg23138872 | TCAF1 |
| cg11471498 | BUB1 |
| cg16320684 | HAUS3 |
| cg01311909 | SFRS2IP |
| cg08623971 | PRSS3 |
| cg25365217 | VTI1A |
| cg20725500 | MAEL |
| cg00653017 | ITCH |
| cg11220060 | KLF1 |
| cg24161613 | MAML1 |
| cg13240253 | FOXP1 |
| cg27421385 | RNF145 |
| cg10640064 | C9orf156 |
| cg19781863 | MON2 |
| cg20987153 | AVEN |
| cg15186333 | LRRC69 |
| cg23145382 | TNRC6B |
| cg00151565 | RC3H1 |
| cg07330481 | ARL5C |
| cg01268901 | WNT5B |
| cg05725404 | NDRG4 |
| cg13610910 | PEX3; ADAT2 |
| cg01933778 | TEX10 |
| cg10932166 | ZFHX3 |
| cg02654372 | KCNQ5 |
| cg15448681 | BAZ2B |
| cg05981968 | PSEN1 |
| cg10349674 | CER1 |
| cg17006282 | RPL36 |
| cg11625005 | TERT |
| cg11169814 | OXCT1 |
| cg19731777 | ALKBH3-AS1 |
| cg12836863 | BRCA2 |
| cg12218359 | CBX7 |
| cg07584910 | ANAPC5 |
| cg19760734 | TACC1 |
| cg05876416 | FAM173B |
| cg00234736 | ELMO1 |
| cg21243612 | C9orf6 |
| cg01779806 | ATP10B |
| cg18105979 | FLT4 |
| cg02214878 | RIT2 |
| cg24736471 | SHC4; EID1 |
| cg08484423 | PARD3 |
| cg26174797 | C2orf53 |
| cg24582618 | VTI1A |
| cg27418687 | POPDC3 |
| cg23091723 | KIAA0319L |
| cg26313511 | ZNF148 |
| cg07895657 | PANX2 |
| cg14097631 | TLN2 |
| cg01174708 | ACACA |
| cg22390660 | P3H2; P3H2-AS1 |
| cg12724001 | RUNX1 |
| cg20642011 | NT5C3A |
| cg11146062 | ARID4B |
| cg01821018 | TACSTD2 |
| cg10593472 | ATG2B |
| cg21694373 | BLOC1S5-TXNDC5 |
| cg06198925 | NIPBL |
| cg22161147 | AFAP1; LOC84740 |
| cg21021332 | MIR6130 |
| cg21147040 | HHAT |
| cg15212455 | POU6F2 |
| cg19992375 | RASSF3 |
| cg23835821 | SULT2A1 |
| cg11427310 | TUBA 1A |
| cg06235424 | CTTNBP2 |
| cg16549361 | CEACAM6 |
| cg26160460 | MIR181B1; MIR181A1 |
| cg02734358 | GPRIN3 |
| cg20040691 | ASB4 |
| cg21695771 | COX7A1 |
| cg00695458 | TRAPPC9 |
| cg23388763 | ZNF146 |
| cg04020590 | GRTP1 |
| cg18127680 | LPP |
| cg11161318 | CYP20A1 |
| cg24908186 | SH3BP5 |
| cg01264438 | GRXCR1 |
| cg00625670 | MGC27382 |
| cg19285539 | SEPT3; WBP2NL |
| cg25068991 | ZNF638 |
| cg20955836 | BMP7 |
| cg09738410 | TRPM5 |
| cg19411084 | PDSS2 |
| cg02747823 | RBM20 |
| cg09969919 | FOXP2 |
| cg16259171 | DNAJB13 |
| cg02392667 | ANKRD46 |
| cg22363621 | NR2C1 |
| cg01389234 | ZBTB20 |
| cg16437904 | MAPKAP1 |
| cg05054124 | ATP6V1H |
| cg12723059 | SLC9B2 |
| cg10922264 | COL20A1 |
| cg16445041 | PIBF1 |
| cg16519495 | ENAH |
| cg19643792 | PTPN12 |
| cg17034181 | SLC30A7 |
| cg04845545 | ZMYND11 |
| cg00997754 | WWP2 |
| cg17550299 | ARHGAP39 |
| cg07986378 | ETV6 |
| cg04926736 | ARID5B |
| cg22437221 | FYB |
| cg17040092 | NCOA2 |
| cg05856951 | HMOX2 |
| cg12647491 | SLK |
| cg01638193 | RAD51L1 |
| cg01916962 | DNAJC5 |
| cg24489015 | LPO |
| cg16579043 | WASF3 |
| cg17896683 | DOCK5 |
| cg11583863 | PPP1R11 |
| cg20029201 | BCL9L |
| cg14136101 | SNX25 |
| cg19101624 | ALG6 |
| cg20421983 | LPPR5 |
| cg14215483 | SLC35A3 |
| cg19714723 | CDH18 |
| cg06773306 | LAMP1 |
| cg12255123 | EPB41L5 |
| cg03551401 | ADCY8 |
| cg12000995 | KRTCAP3 |
| cg08259307 | ZMYND11 |
| cg04895360 | NPAS3 |
| cg09999719 | IL1RAP |
| cg04354845 | GLT6D1 |

TABLE 6
Extragenic Markers Consolidated

Extragenic
markers - Used
in Algorithm
Development
cg10163508
cg00631551
cg01699998
cg12308770
cg16549063
cg17731069
cg00156330
cg07863545
cg10037749
cg13215579
cg22773231
cg03964954
cg18571488
cg06070817
cg26026951
cg15572235
cg26373582
cg15979885
cg27614666
cg21828559
cg18578690
cg11347946
cg04587141
cg02174133
cg20454464
cg12143028
cg04526584
cg04196263
cg07030646
cg12081070
cg23330928
cg05031851
cg01799359
cg03073189
cg16334555
cg12520929
cg17454247
cg24499764
cg07617678
cg04395970
cg16613631
cg03489427
cg27102141
cg22045256
cg01780781
cg12945611
cg02160323
cg13315609
cg16826168
cg05593139
cg24016690
cg19341425
cg10367939
cg13666174
cg21136104
cg06203009
cg10843280
cg00543415
cg12918536
cg19222397
cg17489635
cg13474332
cg19828063
cg18981569
cg11737757
cg22534288
cg11826726
cg26102435
cg11861487
cg10809252
cg20604028
cg08628010
cg17864015
cg07505327
cg08835755
cg16058196
cg09145882
cg05624577
cg14701108
cg05785038
cg25178900
cg15079483
cg21279677
cg24331722
cg14662218
cg03995102
cg12592387
cg11546554
cg01134758
cg18908062
cg10124079
cg05089925
cg23948843
cg10678749
cg21776682
cg23901212
cg20932630
cg17379749
cg14654363
cg08471498
cg04739153
cg13018639
cg24621754
cg14214257
cg06094776
cg09547570
cg24400656
cg08781146
cg04071630
cg16557792
cg01969403
cg23680067
cg20961509
cg20005578
cg13309071
cg23492823
cg02639223
cg19536605
cg07656520
cg24650171
cg03668602
cg13708803
cg16703660
cg16201634
cg21052905
cg12606317
cg23737109
cg24032030
cg21039341
cg11505731
cg20355311
cg09590377
cg10228304
cg26044670
cg21583986
cg08200446
cg07195296
cg21708703
cg16153919
cg07744798
cg12448977
cg18804499
cg01199628
cg25544413
cg26570550
cg01680081
cg14449209
cg03625007
cg09368827
cg11296421
cg09596391
cg08048268
cg07018435
cg07790752
cg10242172
cg02536698
cg21394171
cg09039561
cg14167603
cg00071446
cg02052531
cg01616085
cg07292773
cg21155111
cg23609929
cg08657654
cg03431447
cg00019351
cg06310633
cg16232058
cg02756989
cg17626683
cg08679638
cg25432371
cg04938830
cg05506959
cg08326079
cg25949806
cg12350164
cg08710469
cg26144909
cg25474687
cg09947625
cg22759516
cg20786670
cg13605781
cg10067942
cg04747834
cg15773072
cg04871472
cg15349886
cg24087404
cg16523364
cg01214923
cg10804656
cg04375046
cg14947623
cg00442205
cg19062298
cg24561419
cg02098816
cg07421597
cg19508726
cg16661769
cg16058195
cg23491387
cg25801034
cg06585645
cg13557337
cg14454338
cg16236009
cg19395684
cg03534031
cg13105425
cg15444358
cg11283860
cg15245556
cg10168494
cg22114896
cg22509807
cg06055561
cg02179707
cg26074499
cg14089267
cg08576856
cg23001918
cg01277599
cg15931375
cg17683100
cg22521707
cg26237810
cg15153114
cg23235671
cg24530489
cg18062092
cg17602206
cg02851625
cg15498294
cg11168104
cg18340948
cg08451797
cg23951776
cg11188572
cg13908477
cg06578342
cg24971112
cg12614325
cg07264726
cg24460235
cg01033191
cg17174814
cg22417827
cg16153601
cg00813343
cg23829273
cg13667488
cg05442234
cg11169363
cg25468555
cg09188096
cg04201021
cg26911448
cg18419576
cg08727218
cg10939445
cg18617411
cg07535244
cg14395298
cg15368732
cg13666822
cg11829486
cg07184321
cg23122321
cg16066205
cg08651677
cg04080417
cg19286744
cg27284586
cg19063162
cg23821954
cg03785755
cg00953809
cg04604259
cg27298420
cg27609375
cg08711711
cg15782771
cg04015057
cg11070274
cg19488431
cg01256877
cg16045838
cg14294215
cg01699762
cg21710377
cg06573787
cg15443223
cg22889444
cg03475293
cg02277646
cg12893905
cg00460983
cg04597753
cg01796038
cg13171679
cg12271668
cg12485572
cg06931676
cg15321570
cg21312057
cg02255986
cg04864378
cg15960490
cg16579144
cg02739429
cg22790013
cg21917512
cg05232371
cg13565129
cg16271486
cg13160166
cg01640660
cg04897646
cg27127773
cg27023252
cg24031760
cg16320141
cg16141338
cg12695537
cg18774117
cg02661473
cg05370462
cg03759229
cg05407003
cg07412315
cg19267910
cg11193213
cg22265441
cg13529695
cg13423759

In embodiments, the genomic loci have an AUC (with 95% CI) greater than 0.70, 0.75, 0.80 0.85, 0.86, 0.87, 0.88, 0.89, 0.90, 0.91, 0.92, 0.93, 0.94, 0.95, 0.96, 0.97, 0.98, or 0.99. In embodiments, the genomic loci have an AUC (with 95% CI) of 1.00.

AUC integrates sensitivity and specificity values and gives a more precise indication of the accuracy of the test. AUC (with 95% CI) indicates an AUC with a statistically significant 95% confidence interval. An AUC of ≥0.70 indicates a clinically useful test. In embodiments, the genomic loci are selected from the algorithms having an AUC (with 95% CI), ≥0.8800, 0.8900, 0.9000, 0.9100, 0.9200, 0.9300, 0.9400, 0.9500, 0.9600, 0.9700, 0.9800, or 0.9900. In embodiments, the genomic loci are selected from the algorithms having an AUC (with 95% CI) of 1.0000. In embodiments, the genomic loci are selected from algorithms with a sensitivity and/or specificity of ≥0.8700, 0.8800, 0.8900, 0.9000, 0.9100, 0.9200, 0.9300, 0.9400, or 0.9500.

In embodiments, the genomic loci are selected using one or more of the different AI platforms.

The results presented herein confirm that in an independent validation group based on the differences in the level of methylation of the cytosine sites between AD and normal cases throughout the whole human genome, the predisposition to or risk of having AD can be determined.

The genomic loci reported enable targeted screening studies for the prediction and detection of AD based on cytosine methylation throughout the genome. In embodiments, the genomic loci are used in many different combinations to predict, detect, or diagnose AD in a subject. In embodiments, the genomic loci are used to determine or calculate the risk or predisposition of a patient to having AD at any time in an adult subject or an elderly subject.

In embodiments, the genomic loci for predicting, detecting, or diagnosing AD include cg19760734 (TACC1), cg05876416 (FAM173B), cg00234736 (ELMO1), cg21243612 (C9orf6), cg24040188 (RBBP8).

In embodiments, the plurality of Alzheimer indicator genes includes brain biopsy differentially expressed genes along with demonstrated significant methylation changes. Examples of such genes include at least one or any combinations of RNPS1, CLEC4G, NBL1, BTBD3, C16orf58, DPYSL3, KLF6, MXI1. FRMD4A, GSTM1, SHF, IFIT3, STX6, SLC35F3, CDC14A, COPS7A, IFI16, ALDH2, HS3ST2, VAC14, GNA12, SYNJ1, NPAS1, CAPN2, PLCB1, HCG9, SYT7, APC, SLC47A1, GPR98, TOR1AIP1, ACHE, GNA13, RALB, GFOD2, SP110, CHD5, DPY19L1, WASF2, FDPS, SLC1A2, DDX21, MUTED, ATP6VOE1, PPIL5, ECH1, B4GALNT1, KBTBD8, SEC31A, DYNLT1, CEBPB, LRP4, RASSF4, TRIM6, SLC25A11, PLD3, IMP4, PPME1, RUNDC3B, NCDN, KIAA1712, MRPS11, ACTR1A, MRPS12, PKIB, and ASB3.

In embodiments, the AD indicator genes that are also CpG biomarkers in genes previously believed to be linked to brain injury include C11orf87, FBXL16, GABRA5, GNG13, GPM6A, GRM4, HPCA, KCNN1, KLHL1, LRTM2, NR2E1, SLC17A7, SLC1A2, SNCB, SOX1, and SYNPR that were identified as being epigenetically dysregulated in our circulating cf DNA analysis.

In embodiments, the method further includes a step of further comprising identifying a subject having a mild cognitive impairment and applying the method to determine the risk of Alzheimer's disease for the subject having mild cognitive impairment.

In embodiments, an AI program for calculating the risk of AD based on cf DNA methylation analysis executing at least part of the method is provided.

In embodiments, a method for diagnosing AD or determining susceptibility to AD is provided. The method includes steps of obtaining a biological sample from a target subject, extracting cf DNA from the biological sample, and performing cytosine methylation analysis of genes in cf DNA. In embodiments, the biological sample is blood. A trained neural network is applied to determine if the target subject is at risk for or has AD. Characteristically, the trained neural network is trained from genome-wide methylation test sets that include a first group of testing subjects having AD and a second group of test subjects not having AD diagnosed my current antemortem tests including clinical history and physical exam, psychological testing, and imaging techniques including MRI. Post-mortem confirmation of the diagnosis can further be achieved by pathological examination of the brain specimens to identify the characteristic histological changes that are the gold standard for confirmation of AD. The genome-wide methylation is restricted to a plurality of AD indicators genes. The details and examples for such a plurality of AD indicators genes are set forth above.

In embodiments, the method further includes a step of treating the target subject for Alzheimer's Disease if the target subject is identified as being at risk. In a refinement, the target subject is treated after proper clinical evaluation for Alzheimer's Disease if the target subject is identified as being at risk in a clinical trial. Early and accurate diagnosis is now regarded as critical for interventions for mitigating the disease, prolonging productive years, and the identification of appropriate subjects for early intervention pharmacological trials.

In embodiments, gene methylation analysis is performed genome-wide. Some genes have been reported to be differently expressed in the brains of patients who died of AD. In a refinement, the target subject is identified as having or being at risk for or has AD if there is a methylation difference in one or more CpGs in one or more genes in the plurality of previously identified and AD indicators described herein from those of control subjects not having AD. Methylation levels are generally expressed as (beta) β-values. As per Illumina Corporation, which manufactures the assay probes used, the β-value is defined as an estimate of the methylation level using the ratio of fluorescent intensities between fluoroscopic probes binding to methylated and unmethylated cytosine loci. β-value=Methylated allele intensity (M)/(Unmethylated allele intensity (U)+Methylated allele intensity (M). Thus, for each cytosine locus, the average β-value is calculated for the AD group and also for the control group. The absolute percentage difference in methylation levels-increased (hypermethylated) or decreased (hypomethylation) can be determined. Conversely, the fold change in methylation level in AD cases relative to controls e.g., >1.5 fold or >2.0 fold can be determined.

In embodiments, the method includes a further step of identifying a subject having mild cognitive impairment and applying the method to determine the risk of AD for the subject having mild cognitive impairment as DNA methylation changes are known to precede the development of clinical changes.

In embodiments, an AI program executing on a computing device for calculating the risk of AD based on cf DNA methylation analysis executing at least part of the method is provided.

Treatment. In embodiments, the methods described herein further include a step of treating the target subject for Alzheimer's Disease as the target subject is identified as being at increased risk. In embodiments, the target subject is treated in a clinical trial for Alzheimer's Disease if the target subject is identified as being at risk in a clinical trial.

10106| AD can be treated by medication including Aduhelm, Aricept, Razadyne, Exelon, Memantine, Namzaric, and a combination thereof. Aduhelm (aducanumab) is an approved drug for reducing amyloid beta plaques in the brain. Aricept (donepezil) is an approved drug for treating all stages of AD, mild, moderate, and severe. Razadyne (formerly Reminyl, galantamine) is for treating mild to moderate AD. Excelon (rivastigmine) is also for treating mild to moderate AD. Memantine (Namenda) treats moderate to severe AD. Namzaric is a mix of Namenda and Aricept and is for treating patients with moderate to severe AD who already take the two drugs separately.

Aricept, Razadyne, and Exelon work by inhibiting the breakdown of acetylcholine in the brain, which is important for memory and learning. Memantine works by changing the amount of glutamate, a brain chemical that plays a role in learning and memory. Brain cells in AD patients give off too much glutamate, so Memantine is able to keep the levels of the chemical in check.

The methods described herein enable early diagnosis of AD since methylation changes are known to occur early in or possibly involved in the initiation of the disease process and provide AD patients with the benefits of access to the right services and support to help them take control of their condition, live independently in their own home for longer, and maintain a good quality of life for themselves, their family, and care-givers. Good quality of life in the early phases of the illness can be maintained for several years. Early diagnosis enables AD patients to access available treatments that may improve their cognition and enhance their quality of life. Moreover, early diagnosis allows caregivers time to adjust to the changes in the AD patient and adapt to their role as a caregiver. Early diagnosis of AD allows for lifestyle changes that can slow or prevent the development of future diseases. Vascular disease and dementia syndromes have many shared risk factors including hypertension, type 2 diabetes, smoking, and poor diet and exercise habits.

Microarray. Differential methylation can be analyzed using a microarray system. Nucleic acids can be linked to chips, such as microchips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Binding to nucleic acids, such as cf DNA, on microarrays can be detected by scanning the microarray with a variety of laser or charge-coupled device (CCD)-based scanners, and extracting features with software packages, for example, Imagene (Biodiscovery, Hawthorne, CA), Feature Extraction Software (Agilent), Scanalyze (Eisen, M. 1999. SCANALYZE User Manual; Stanford Univ., Stanford, Calif. Ver 2.3.2.), or GenePix (Axon Instruments). A full panel of loci would include one or more genomic loci listed in Table 1B, 2B, 3B, or 4B that have been shown individually to be potentially clinically useful tests AUC≥0.70.

Kits. Kits for predicting and diagnosing AD based on methylation of CpG loci in nucleic acids from any source whether cellular-based or extracellular, such as circulating cf DNA, are described. The kits can include the components for extracting cf DNA from the biological sample, the components of a microarray system, and/or for analysis of the differentially methylated genomic sites.

Biomarker diagnosis and prediction of AD as described herein can lead to early and accurate diagnosis and thus facilitate the management and long-term care objectives. Given the evidence of an increase in AD cases, accurate biomarkers are a critical necessary complement to any effective treatment strategy.

Methods disclosed herein include predicting, detecting, or diagnosing AD and/or calculating risk or disposition to developing AD. The methods described herein can be used in the prevention and/or treatment (including mitigating or alleviating symptoms) of patients at an early stage of the development of other diseases. Subjects or patients in need of (in need thereof) predicting, diagnosing, and/or treating are subjects that may have AD and/or need to be diagnosed and treated.

As will be understood by one of ordinary skill in the art, each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. Examples of steps that do not materially affect an embodiment of the subject matter described herein include steps that do not materially affect the detection, prediction, or diagnosis of AD, or do not materially affect the prevention or treating of AD of a patient.

In addition, unless otherwise indicated, numbers expressing quantities of ingredients, constituents, reaction conditions, and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

When further clarity is required, the term “about” has the meaning reasonably ascribed to it by a person skilled in the art when used in conjunction with a stated numerical value or range, i.e. denoting somewhat more or somewhat less than the stated value or range, to within a range of +20% of the stated value; +15% of the stated value; +10% of the stated value; +5% of the stated value; +4% of the stated value; +3% of the stated value; +2% of the stated value; +1% of the stated value; or ±any percentage between 1% and 20% of the stated value.

The following examples illustrate the various embodiments of the present invention. Those skilled in the art will recognize many variations that are within the spirit of the present invention and scope of the claims.

### EXAMPLES

### Example 1

Brief Summary Despite extensive efforts, significant gaps remain in our understanding of Alzheimer's disease (AD) pathophysiology. Novel approaches using circulating cell-free DNA (cf DNA) analysis have the potential to revolutionize our understanding of neurodegenerative disorders. In addition, there is a great need for accurate non-invasive AD biomarkers. A genome-wide methylation profiling of cf DNA from AD patients was performed and compared to cognitively normal controls. Six Artificial Intelligence (AI) platforms were utilized for the diagnosis of AD while enrichment analysis was used to help elucidate the molecular pathogenesis of AD. A total of 3684 CpGs were significantly (adjusted p-value<0.05) differentially methylated in AD versus controls. All of the six AI algorithms evaluated achieved high predictive accuracy (AUC=0.949-0.998) in an independent test group. For example, Deep Learning (DL) achieved an AUC (95% CI)=0.99 (0.95-1.0), with 94.5% sensitivity and specificity using intragenic CpG markers. Similar predictive accuracies were achieved using extragenic markers only. CpG markers both within and outside of genes were identified by AI. Subanalyses of CpGs in genes previously known to be expressed in the brain or have been previously linked to AD were also performed. Enrichment in the Calcium signaling pathway. Glutamatergic synapse, Hedgehog signaling pathway, Axon guidance, and Olfactory transduction in those patients suffering from AD are highlighted. Further, numerous epigenetically altered cf DNA genes were previously reported to be differentially expressed in the brain of AD sufferers are described. This is the first reported genome-wide DNA methylation study using cf DNA to detect AD.

Introduction. Alzheimer's disease (AD) is the leading cause of severe dementia, however, the etiological mechanisms of the disease have yet to be elucidated. The spectrum of putative AD pathophysiology is wide and expanding.¹ Mechanistic information on AD could yield clinical benefits. For example, information on disease pathogenesis could lead to the development of novel biomarkers and therapeutic targets. Given the long latency period and time course of AD, even in the absence of definitive treatment, therapies that slow disease progression or reduce the dementia burden can significantly improve the quality of life and yield substantial healthcare savings².

Epigenetic mechanisms regulate gene expression independent of DNA sequence changes.³ DNA methylation is the most commonly studied epigenetic mechanism⁴ and is known to play a significant role in AD pathogenesis while offering the prospect of targeted correction.⁵ Currently, circulating cf DNA, so-called ‘liquid biopsy’, is being used extensively in the study of cancer evolution,^{6, 7} cardiomyocyte death,⁸ and for non-invasive biomarkers for transplant rejection⁹⁻¹¹. Circulating nucleic acid levels were found to be elevated in the plasma of AD patients, the plasma of a transgenic mouse model of AD, and in the culture medium of cells treated with amyloid-β¹² raising interest in its potential as AD biomarkers. Theoretically, neuronal, vascular, and inflammatory responses along with the anatomical and functional changes in the brain of AD sufferers, could be non-invasively monitored¹³, in the future, given the fact that the DNA of cells from brain tissues contribute to the pool of circulating cf DNA.

There is intense research interest in the development of non-invasive blood-based biomarkers for AD. Potential advantages include reduced reliance on invasive or expensive diagnostic techniques such as lumbar puncture, PET scans, and MRI imaging techniques.¹⁴ Artificial Intelligence (AI) including Deep Learning (DL) offers distinct advantages in the analysis of the vast troves of biological data generated from omics experiments such as DNA-methylation.¹⁵⁻¹⁸

In this study, methylation profiling of circulating cf DNA collected from individuals suffering from AD was performed and compared to cognitively healthy controls. Using AI analysis, the accuracy of putative cytosine (CpG) epigenetic markers for AD diagnosis was analyzed. Pathway analysis was used to further understand the molecular pathogenesis of AD.

Methods and Materials. The study was approved by the Human Investigation Committee of William Beaumont Hospital, Royal Oak, Michigan, USA (IRB #2017-214). Written consent was obtained from study participants or their legal representatives. A total of 52 subjects were prospectively recruited (26 AD cases and cognitively healthy 26 controls). The diagnosis of AD was based on existing clinical and laboratory criteria according to NINCDS-ADRDA.¹⁹ Blood samples were collected from each subject in Streck Cell-Free DNA BCT® tubes. This minimizes further dilution and confounding from DNA that is released due to leukocyte lysis at the time of collection and during storage.²⁰ The samples were processed within 24 hours of the blood draw. For initial sample processing, specimens were centrifuged for 15 minutes at 3000×g and the plasma was aliquoted into 2.0 ml Eppendorf Safe-Lock micro-centrifuge tubes without disturbing the buffy coat and subsequently stored at −80° C. for further processing.²¹ The cf DNA was extracted from plasma using the QIAamp circulating nucleic acid kit (Qiagen Cat #55114) and a manual vacuum as per the manufacturer's standardized protocol.

DNA methylation profiling. The extracted cf DNA was subjected to bisulfite conversion using the EZ DNA Methylation Kit (Zymo, USA) per the manufacturer's instructions and the bisulfite converted DNA was eluted using 10 μl of elution buffer.²² Following bisulfite conversion, the Illumina Infinium MethylationEPIC BeadChip arrays for methylation profiling as per the manufacturer's instructions were performed. The vacuum-dried BeadChips were imaged immediately on an Illumina iScan System (Illumina, Inc.).

Statistical and bioinformatic analysis. All data analysis was performed using R version 4.1.1. Raw EPIC array data were processed using the package “minfi”. Noob normalization was used to normalize the signal.

Outlier detection: Probe values not passing the detection threshold were marked as missing. Sex chromosome methylation probes were removed from the analysis to avoid gender-specific methylation bias and to avoid the possible difficulties of having matched X and Y chromosome methylation markers caused by the epigenetic inactivation of one X chromosome in females 23. The fraction of missing probe values was estimated for all samples and those with the fraction more than two standard deviations (95% confidence) away from the mean were deemed outliers. The K nearest neighbor algorithm with default parameters implemented in the “impute” package was used to impute missing values. Probes with variability higher than 0.01 across all samples were retained for further analysis. Immune cell-type deconvolution was performed using the minfi package.

Variance inflation: The proportion of granulocyte markers was identified as a strongly inflated covariate and correlated with other variables (Bcell, CD4T, CD8T, NK). After the removal of the inflated covariate (granulocyte markers), other variables did not show any correlation with each other.

The methylation beta values were transformed into M values and robust linear regression (M˜b0+b1*ConditionAD+b2*Age+b3*GenderFemale+b4*BMI+b5*CD8T+b6*CD4T+b7*NK+b8*Bcell+b9*Mono+error) as implemented in the “limma” package was used to establish differentially methylated cytosines. The reported fold change (log FC) is the value of coefficient b1.

Variance inflation. The regression model included concurrent medical disorders, age, gender, and BMI as covariates, as well as the cell type proportions of CD8T, CD4T, NK, Bcell, and monocytes. As noted, hemolysis of these cell types can add to the apparent cf DNA pool in plasma. Other estimated immune cell type proportions were found to be colinear with the aforementioned ones and were not included in the model. Fisher's exact test comparing the number of significant hyper-methylated cytosines among all the significant cytosines to the total number of hyper-methylated cytosines among all interrogated cytosines was used to determine the overall trend towards hyper-methylation among significantly differentially methylated cytosines. Similarly, all cytosines were annotated with genomic and CpG island regions, and enrichment of such regions with differentially modified cytosines was tested using Fisher's exact test.

Enrichment analysis. Pathway enrichment analysis was performed by annotating each EPIC array probe with the UCSC reference gene symbol. For each gene, the CpG locus with the lowest overall p-value was retained. The genes were subsequently ranked by negative log transformed p-values and passed to the g: profiler service for enrichment analysis. Next, genes were ranked by the sign of fold change multiplied by negative log transformed p-value and passed to the gene set enrichment function implemented in the clusterProfiler package.

Artificial Intelligence/Deep learning (AI/DL) Analysis. The detailed AI analysis is presented in our prior publications.¹⁸ In brief, the overall CpG markers after normalization in AD subjects as compared to controls were used. DL and five other AI algorithms were used: Support vector machine (SVM), Generalized Linear Model (GLM), Prediction Analysis for Microarrays (PAM), Random Forest (RF), and Linear Discriminant Analysis (LDA) to perform classification and regression analysis.²⁴ The study patients were randomly separated into a ‘training’ group for predictive algorithm development and an independent test group to determine its performance.

Random Forest (RF) is a supervised learning algorithm for classification, regression, and other functions. It is supervised in the respect that the function is inferred from initially labeled training data. A forest of decision trees is randomly created, and the mean prediction of the individual trees is determined. There is a direct correlation between the number of trees in the forest and the accuracy of the results that are generated. The accuracy of the results is increased by increasing the number of trees. RF has several benefits such as being able to work with missing values and analysis of categorical values.⁷³ Support Vector Machine (SVM) is first fed with labeled data (supervised learning) permitting identification of the different groups and from this, it builds a model for distinguishing the groups. Subsequently, when provided with unlabeled fresh data SVM develops models or hyperplanes to separate one group from another. SVM is capable of performing both regression and classification tasks and can handle both continuous and categorical variables.⁷⁴ SVM is resistant to overfitting, which is a risk in the analysis of small datasets. Linear Discriminant Analysis (LDA) reduces the number of features or predictors need to accurately classify and discriminate the groups. This is desirable for the dataset as it starts with close to 900,000 potential features to be used for AD detection. LDA is simple in approach but it still achieves excellent accuracy. The accuracy achieved is similar to that obtained with more complex methods. LDA is based on the identification of a linear combination of variables (predictors) that best separates the two classes (targets) 75. It is closely related to the analysis of variance (ANOVA) and regression analysis which attempts to define an outcome variable based on a combination of explanatory variables. Partitioning Around Medoids (PAM) is a statistical technique for class prediction from gene expression data using the nearest shrunken centroids.^{70, 76} This method identifies the subsets of genes that best characterize each class. Generalized Linear Models (GLMs) are a broad class of models that include linear regression, ANOVA, Poisson regression, log-linear models, and others.^{70, 76} Deep Learning (DL) is a form of representation learning that uses multiple transformation steps to create very complex features. DL is categorized into feed-forward artificial neural networks (ANNs), which use more than one hidden layer (y) that connects the input (x) and output layer (z) via a weight (W) matrix. The weight matrix is expected to minimize the difference between the input and output layers and is considered the best AI approach.^{70, 76}

Modeling & Evaluation: Two-step validation was utilized for these analyses. There were two different data sets: the first was utilized to build the model and test it, and the second one was used to validate the model.

While using the two-step validation method, two different techniques were utilized to find out the best model and calculate the performance metrics: 10-fold Cross-Validation and Bootstrapping.

Ten-fold Cross-Validation: The first data set was split into training the model with a portion of the data and testing the remaining portion on which the performance of the developed model is then determined. Here, the available set of samples was randomly divided into two parts: a training set and a test or hold-out set. The model was fitted on the training set, and the fitted model was used to predict the responses for the observations in the hold-out set. Estimates were used to select the best model and to give an idea of the test error of the final chosen model. The Idea was to randomly divide the data into 10 equal-sized parts. Part 10 was left out, and the model was based on the other 9 parts (combined); and then predictions were obtained for the left-out 10th part. This was done in turn for each part k=1, 2 . . . 10, and then the results were combined. This process was repeated a total of ten times and the average AUC, sensitivity, specificity, and 95% confidence intervals for the test set were calculated. Subsequently, as the validation step, AUC, sensitivity, specificity, and 95% confidence intervals for the validation data set were calculated.

Bootstrapping: The bootstrap is a flexible and powerful statistical tool that allowed the use of a computer to mimic the process of obtaining new data sets, enabling the estimation of the variability of the estimate without generating additional samples. Rather than repeatedly obtaining independent data sets from the population, distinct data sets were obtained by repeatedly sampling observations from the original data set with replacement. Each of these “bootstrap data sets” was created by sampling with replacement and was the same size as our original dataset. As a result, some observations appeared more than once in each bootstrap data set, and some did not appear at all. To estimate prediction error using the bootstrap, each bootstrap dataset was used as the training sample, and the original sample as the test sample. This process was repeated a total of ten times and the average AUC, sensitivity, specificity, and 95% confidence intervals for the test set were calculated. Subsequently, the validation step, AUC, sensitivity, specificity, and 95% confidence intervals for the validation data set were calculated.

To establish the robustness of the predictive algorithms, the biomarker combinations were first developed in a Training group (patient and controls) and the performance was validated in an independent patient Test group of cases and controls.

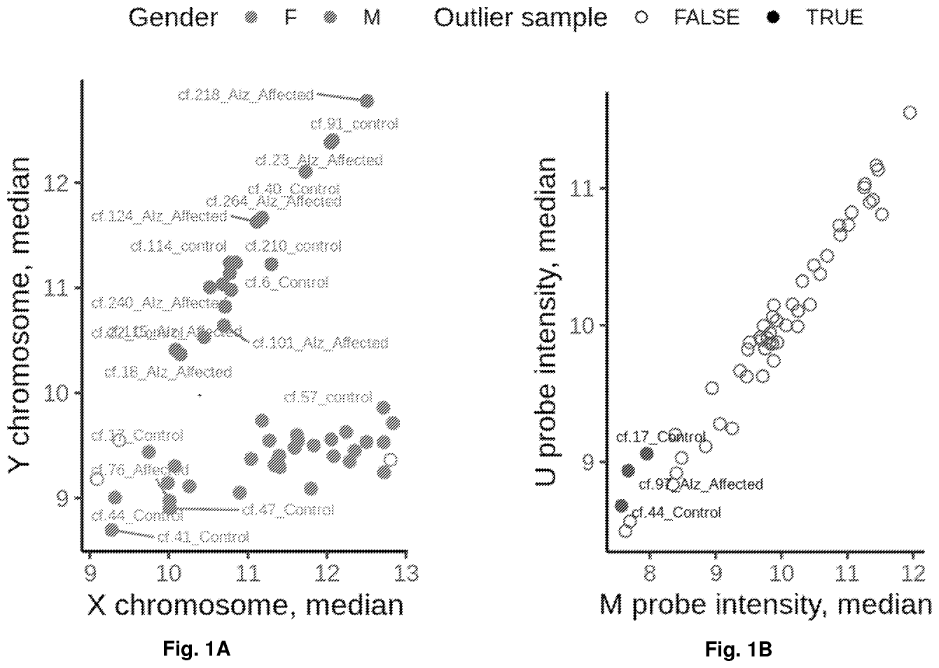

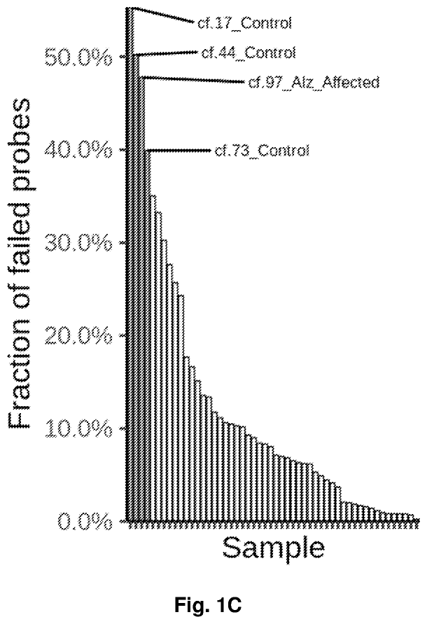

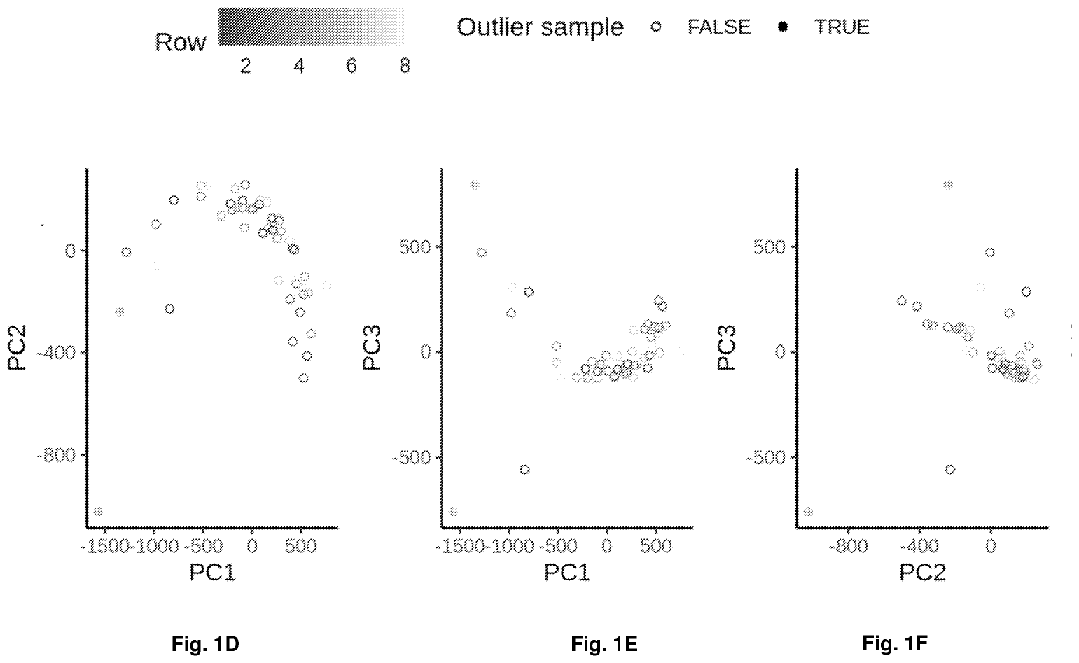

Results. Genome-wide DNA methylation of circulating cf DNA from 26 people suffering from AD was evaluated and compared to 26 cognitively healthy controls. However, one AD subject and three controls were outliers and removed from further analyses (FIGS. 1A-1F). Clinical and demographic details are presented in (Table 7). The mean (SD) age was slightly higher in AD cases [82 (7)] versus controls [79 (9)], p=0.01, and as such, all methylation changes were normalized for age. No other significant differences were noted for all other potential confounders including gender (p=0.52), ethnicity (p=0.48), cardiovascular diseases, or TBI (Table 7). As expected, the Mini-Mental State Exam (MMSE) score was significantly lower for AD cases compared to controls: Mean (SD)=20 (4) versus 29 (1), p<0.001.

TABLE 7
Comparison of demographics and clinical characteristics:
Alzheimer's disease cases vs. normal controls.

| | | | | | q-value | |
|---|---|---|---|---|---|---|
| Parameter | Cases | | Controls | | (FDR) | |
| Number of patients | 26 | | 26 | | — | |
| Age [Mean (Standard deviation)] | 82.45 | (7.11) | 79.26 | (9.63) | 0.01 | (W) |
| Gender (%) | | | | | | |
| Females | 50 | | 65.38 | | 0.52 | (W) |
| Males | 42.30 | | 34.61 | | | |
| Data unavailable | 7.69 | | 0 | | | |
| Race (%) | | | | | | |
| Non-Hispanic | 92.30 | | 88.46 | | 0.48 | (W) |
| Hispanic | 0 | | 7.69 | | | |
| Not reported | 7.69 | | 3.84 | | | |
| MMSE Score [Mean (Standard | 20.09 | (4.74) | 28.92 | (1.07) | <0.0001 | (W) |
| deviation)] | | | | | | |
| Stroke (%) | | | | | | |
| Yes | 7.69 | | 7.69 | | 0.11 | (W) |
| No | 80.76 | | 88.46 | | | |
| Data unavailable | 11.53 | | 3.84 | | | |
| Hyperlipidemia (%) | | | | | | |
| Yes | 73.07 | | 65.38 | | 0.52 | (W) |
| No | 19.23 | | 30.76 | | | |
| Data unavailable | 7.69 | | 3.84 | | | |
| Hypertension (%) | | | | | | |
| Yes | 65.38 | | 61.53 | | 0.40 | (W) |
| No | 26.92 | | 34.61 | | | |
| Data unavailable | 7.69 | | 3.84 | | | |
| Diabetes (%) | | | | | | |
| Yes | 19.23 | | 26.92 | | 0.14 | (W) |
| No | 73.07 | | 69.23 | | | |
| Data unavailable | 7.69 | | 3.84 | | | |
| Yes | 23.07 | | 3.84 | | 0.52 | (W) |
| No | 69.23 | | 92.30 | | | |
| Data unavailable | 7.69 | | 3.84 | | | |
| BMI [Mean (Standard deviation)] | 26.43 | (4.21) | 25.81 | (5.03) | 0.40 | (W) |

Traumatic Brain Injury (TBI) (%)
W—Wilcoxon Mann Whitney test

Abundance of significantly methylated cytosines: Based on the p-value histogram, a significant number of CpG methylation changes having a significance value less than 0.05 (FIG. 2A) was identified, which is also reflected in the volcano plot (FIG. 2B). Overall, the study yielded a significantly higher number of hypermethylated CpGs (FIG. 2C). A statistically significant change in methylation (adjusted p<0.05) in a total of 3,684 CpGs was identified, among which 2,729 CpGs were found to be hypermethylated and the remaining 955 CpGs were hypomethylated in AD. 920 differentially methylated regions (DMRs) (adjusted p<0.05) were also identified, among them, 854 DMRs were hypermethylated and the remaining 66 DMRs were hypomethylated.

AI analysis was performed in an unbiased fashion. All CpGs that met technical quality criteria (irrespective of statistical p-values) were considered in the identification, ranking of CpG biomarkers, and for the subsequent development of predictive algorithms.

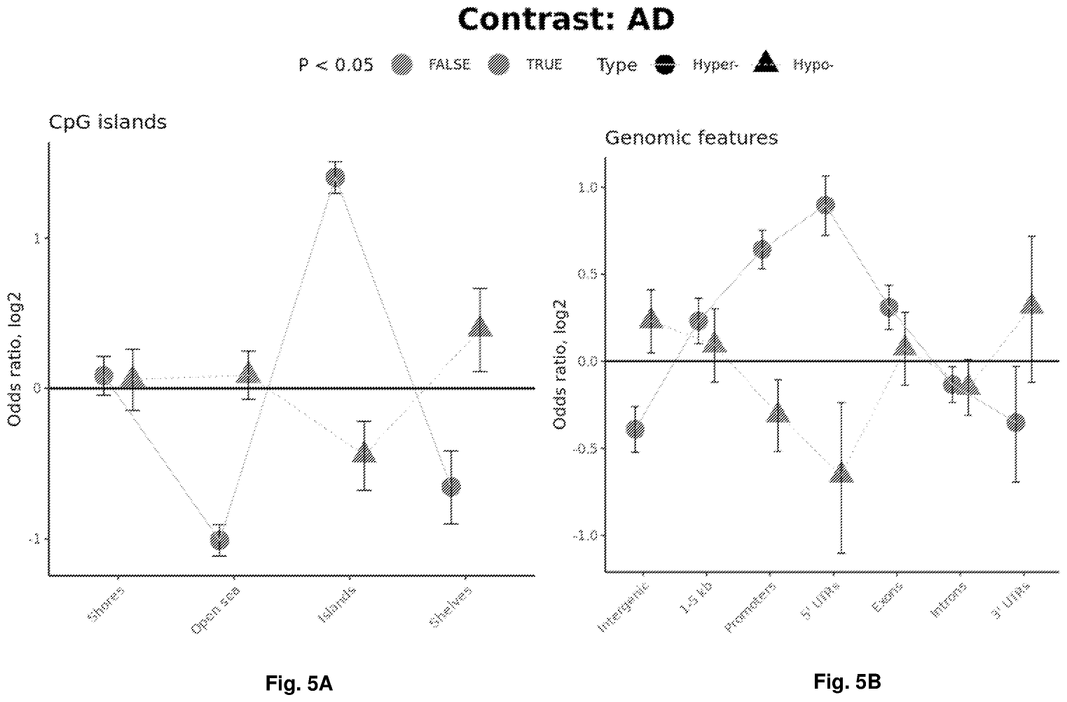

Enrichment analysis. Based on the enrichment of CpG regions, the CpGs on the islands were hypermethylated with an FDR p=1.4×10⁻¹³⁷. Based on the genomic regions, CpGs in the intergenic region were the most hypermethylated with FDR p=5.1×10⁻⁸³ followed by those in the promoter regions in AD cf DNA (FDR p=8.8×10⁻²⁹). Further details are provided in FIGS. 5A and 5B.

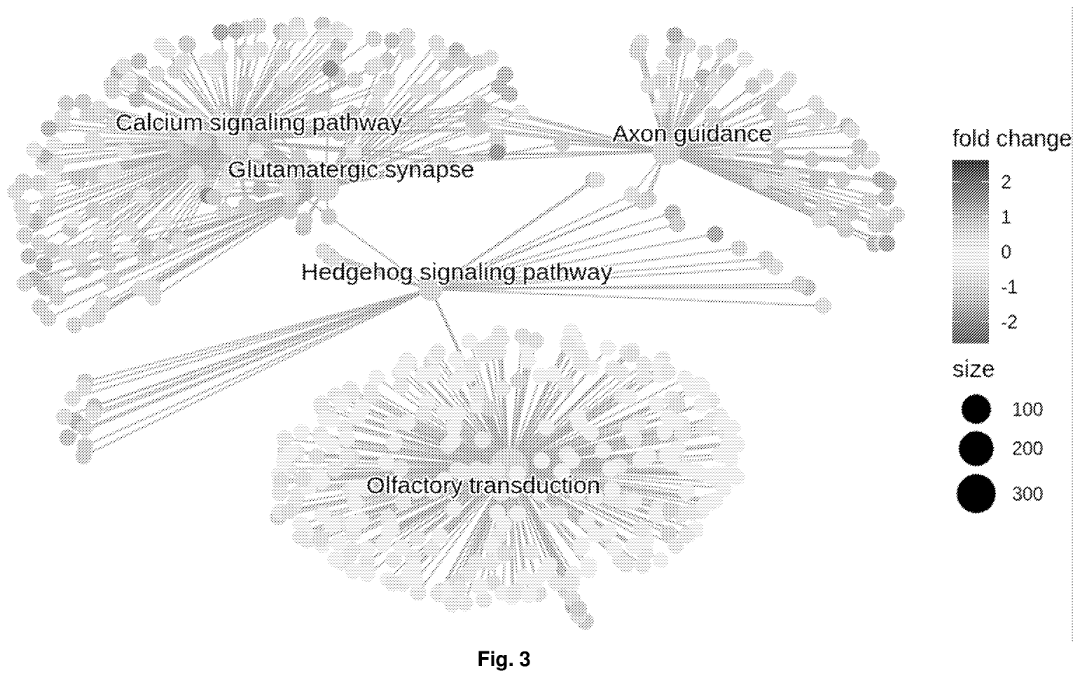

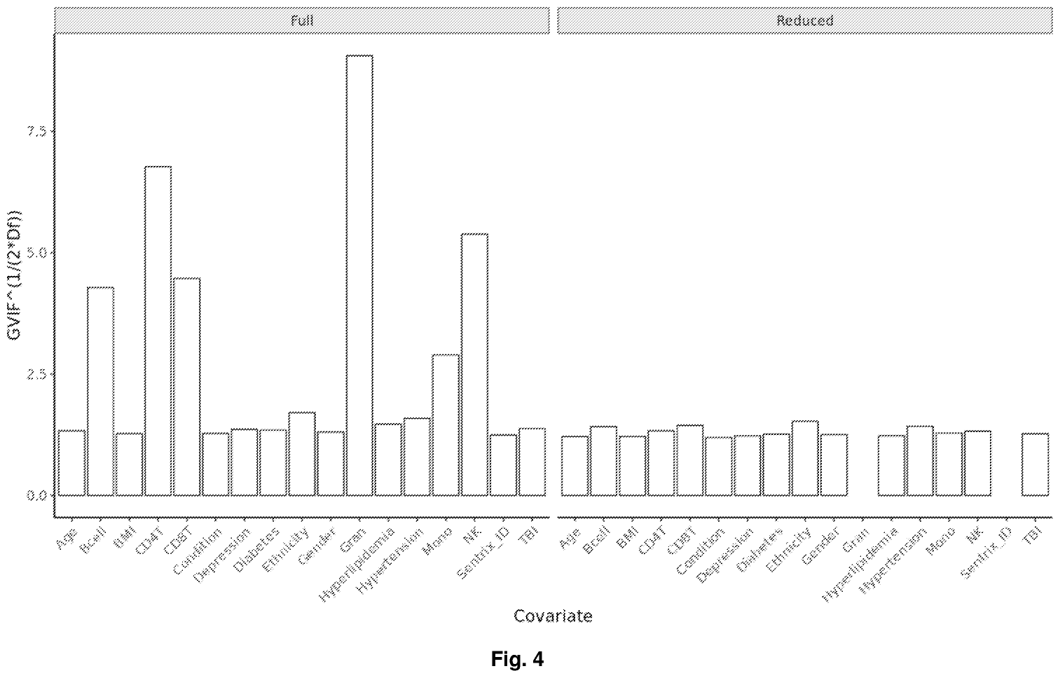

Disease and functional enrichment: Gene ontology analysis was used to identify biological processes and/or molecular functions associated with the differentially methylated genes. Analysis identified the Calcium signaling pathway (CpG set size=227) (q=9.77×10⁻⁰⁵), Glutamatergic synapse (CpG set size=109) (q=9.77×10⁻⁰⁵), Hedgehog signaling pathway (CpG set size=52) (q=0.00032), Axon guidance (CpG set size=174) (q=0.00032) and Olfactory transduction (CpG set size=387) (q=0.00044) as the top 5 perturbed networks. The cluster of genes encompassing these mechanisms is depicted in FIG. 3. Detailed information of KEGG pathway identifiers, pathway description, statistical significance, and the enriched genes list is provided in Table 8.

TABLE 8
List of Significant Pathways

| CF DNA Methylation in AD/EPIC Arrays | | | | | | | | | | | |
|---|---|---|---|---|---|---|---|---|---|---|---|
| | | Set | Enrichment | | | | | | Leading | | methylated |
| ID | Description | Size | Score | NES | pvalue | p.adjust | qvalues | rank | edge | core_enrichment | genes |
| hsa04020 | Calcium | 227 | 0.3341 | 1.998 | 6.46343 | 0.00012 | 9.77412 | 7473 | tags = 65%, | CACNA1C/MYLK/PLCD1/GRIN2C/PTG | CACNA1C/MY |
| | signaling | | 02936 | 19537 | 412516 | 8597 | E−05 | | list = 39%, | ER3/FGF19/TACR1/FGF3/STIM1/GNA | LK/PLCD1/GRI |
| | pathway | | | 6 | 302e−7 | | | | signal = 40% | Q/FGFR2/ADRA1D/PLCB1/GRIN2A/RY | N2C/PTGER3/ |
| | | | | | | | | | | R1/DRD5/ADCY9/TBXA2R/CHRM1/MC | FGF19/TACR1/ |
| | | | | | | | | | | U/GRIN2D/NOS2/SLC8A3/PDGFRA/CA | FGF3/STIM1/G |
| | | | | | | | | | | MK2D/FLT4/CHRM3/TPCN2/FGFR4/CA | NAQ/FRFR2/A |
| | | | | | | | | | | MK1/CHRM2/CAMK1D/FGF8/PPP3CB/I | DRA1D/PLCB1 |
| | | | | | | | | | | TPKA/ITPR1/GNAL/CD38/P2RX7/ADO | /GRIN2A/RYR1 |
| | | | | | | | | | | RA2B/PDGFA/ATP2A3/CASQ2/EGFR/S | /DRD5/ADCY9/ |
| | | | | | | | | | | LC8A1/PLCE1/PLCG2/ADRB3/PTGFR/ | TBXA2R/CHR |
| | | | | | | | | | | CALM3/NGF/VEGFC/PLCB4/TPCN1/M | M1/MCU/GRIN |
| | | | | | | | | | | YLK2/ITPKB/ADCY7/GNA11/NTSR1/RY | 2D/NOS2/SLC |
| | | | | | | | | | | R3/PLCB3/CACNA1B/GNAS/PTAFR/P | 8A3/PDGFRA/ |
| | | | | | | | | | | RKCG/FGFR1/RYR2/NTRK2/GRM5/PD | CAMK2D/FLT4 |
| | | | | | | | | | | GFD/CALML3/PDGFC/ATP2B4/ASPH/ | /CHRM3/TPCN |
| | | | | | | | | | | CAMK2B/HGF/PDE1B/ADCY4/ADCY8/ | 2/FGFR4/CAM |
| | | | | | | | | | | P2RX2/LHCGR/EDNRB/OXTR/CAMK2 | K1/CHRM2/CA |
| | | | | | | | | | | G/PHKG1/ERBB2/CALM1/PPP3CC/HT | MK1D/FGF8/P |
| | | | | | | | | | | R5A/PPP3R1/CAMK2A/PLCG1/SPHK2/ | PP3CB/ITPKA/ |
| | | | | | | | | | | CACNA1D/AVPR1A/PRKACA/VDAC1/P | ITPR1/GNAL/C |
| | | | | | | | | | | HKG2/ITPR3/PTGER1/ATP2B1/SPHK1/ | D38/P2RX7/M |
| | | | | | | | | | | FGF18/SLC8A2/PPP3CA/TACR3/KDR/ | COLN1 |
| | | | | | | | | | | LTB4R2/FGFR3/GRM1/P2RX4/VEGFA/ | |
| | | | | | | | | | | ERBB4/CXCR4/MCOLN3/PDE1C/PTK2 | |
| | | | | | | | | | | B/P2RX6/ADCY2/ADCY1/GRIN1/CALM | |
| | | | | | | | | | | 2/ORAI3/MCOLN1/FGF5/PLCD4/HRC/P | |
| | | | | | | | | | | HKB/HTR4/ADRA1A/CHRNA7/FGF2/E | |
| | | | | | | | | | | RBB3/PDGFRB/ADCY3/PLCB2/SLC25 | |
| | | | | | | | | | | A4/HRH1/EGF/AVPR1B/PRKACB/P2R | |
| | | | | | | | | | | X5/TRDN/PDGFB/EDNRA/MYLK3/ADO | |
| | | | | | | | | | | RA2A | |
| hsa04724 | Glutamatergic | 109 | 0.4083 | 2.181 | 7.77021 | 0.00012 | 9.77412 | 7318 | tags = 71%, | SLC1A2/HOMER1/CACNA1C/GRM6/G | SLC1A2/HOM |
| | synapse | | 23314 | 28493 | 931140 | 8597 | E−05 | | list = 38%, | RIN2C/GRIK4/SHANK1/ACDY5/SLC1A | ER1/CACNA1 |
| | | | | 9 | 558e−7 | | | | signal =44% | 6/GRIA4/GNAQ/GNAI1/PLCB1/GRIN2A | C/GRM6/GRIN |
| | | | | | | | | | | /GRM4/GRM7/SHANK2/GNG4/ADCY9/ | 2C/GRIK4/SHA |
| | | | | | | | | | | GRIN2D/JMJD7- | NK1/ADCY5/S |
| | | | | | | | | | | PLA2G4B/GRIK3/GRIN3A/PPP3CB/ITP | LC1A6/GRIA4/ |
| | | | | | | | | | | R1/SLC17A7/GNG13/GNB5/GNG2/GR | GNAQ/GNAI1/ |
| | | | | | | | | | | M3/GNB3/GRIK2/SLC1A1/PLCB4/ADC | PLCB1/GRIN2 |
| | | | | | | | | | | Y6/ADCY7/DLGAP1/PLCB3/GNG12/SL | A/GRM4/GRM |
| | | | | | | | | | | C1A3/GNAS/SLC38A2/HOMER3/PRKC | 7/SHANK2/GN |
| | | | | | | | | | | G/PLD1/GNB4/GRM5/GNB1/ADCY4/AD | G4/ADCY9/GR |
| | | | | | | | | | | CY8/GRIK1/GRM2/PPP3CC/PPP3R1/C | IN2D/JMJD7- |
| | | | | | | | | | | ACNA1D/GRIA2/PRKACA/ITPR3/HOM | PLA2G4B/GRI |
| | | | | | | | | | | ER2/PPP3CA/GRM1/PLA2G4D/KCNJ3/ | K3/GRIN3A/PP |
| | | | | | | | | | | GLUL/ADCY2/ADCY1/GRIN1/SHANK3/ | P3CB/ITPR1/S |
| | | | | | | | | | | SLC38A1/GNAI3/ADCY3/PLCB2/GLS/P | LC17A7/GNG13 |
| | | | | | | | | | | LA2G4A/MAPK1/PRKACB/SLC17A6 | |
| hsa04340 | Hedgehog | 52 | 0.4916 | 2.263 | 4.28743 | 0.00043 | 0.00032 | 5403 | tags = 69%, | KIF7/CDON/CUL3/CSNK1D/EFCAB7/G | KIF7/CDON/C |
| | signaling | | 23895 | 69618 | E−06 | 0697 | 7355 | | list = 28%, | LI3/SCUBE2/GSK3B/BCL2/IQCE/LRP2/ | UL3/CSNK1D/ |
| | pathway | | | 4 | | | | | signal = 50% | SHH/ARRB1/CCND2/CSNK1G2/BTRC/ | EFCAB7/GLI3/ |
| | | | | | | | | | | SMURF2/DISP1/PTCH2/GLI1/EVC2/SU | SCUBE2/GSK3 |
| | | | | | | | | | | FU/DHH/MGRN1/MEGF8/EVC/FBXW11 | B/BCL2/IQCE/ |
| | | | | | | | | | | /ARRB2/CSNK1A1/SMO/PRKACA/IHH/ | LRP2/SHH |
| | | | | | | | | | | HHIP/CSNK1E/SMURF1/SPOP | |
| hsa04360 | Axon | 174 | 0.3397 | 1.967 | 5.2048 | 0.00043 | 0.00032 | 4733 | tags = 47%, | ABLIM1/MYL9/PARD6G/SEMA6D/WNT | ABLIM1/MYL9/ |
| | guidance | | 91057 | 99276 | E−06 | 0697 | 7355 | | list = 25%, | 5A/CXCL12/RGMA/CFL2/PLXNC1/ABLI | PARD6G/SEM |
| | | | | 8 | | | | | signal = 35% | M2/MYL12B/PRKCZ/DCC/GNAI1/UNC5 | A6D/WNT5A/C |
| | | | | | | | | | | C/MYL12A/ROBO1/GSK3B/CAMK2D/E | XCL12/RGMA/ |
| | | | | | | | | | | PHB1/SEMA4B/SLIT1/SRGAP2/ROBO2 | VFL2/PLXNC1/ |
| | | | | | | | | | | /RASA1/LRRC4C/PTK2/PPP3CB/EPHA | ABLIM2/MYL1 |
| | | | | | | | | | | 8/BMP7/SLIT3/ITGB1/PIK3CD/SHH/UN | 2B/PRKCZ/DC |
| | | | | | | | | | | C5A/EFNA1/SSH3/FYN/NRP1/NTN3/N | C/GNAI1/UNC |
| | | | | | | | | | | FATC4/SEMA3G/PLCG2/UNC5D/SEMA5 | 5C/MYL12A/R |
| | | | | | | | | | | 4C/CDK5/SEMA5B/EPHA5/ROBO3/PA | OBO1/GSK3B/ |
| | | | | | | | | | | K2/RHOA/UNC5B/NRAS/NCK2/GDF7/S | CAMK2D/EPH |
| | | | | | | | | | | EMA23B/DPYSL5/EFNA3/NGEF/EPHA4/ | B1/SEMA4B/S |
| | | | | | | | | | | CAMK2B/SEMA6C/WNT5B/NFATC2/S | LIT1/SRGAP2/ |
| | | | | | | | | | | RGAP1/PLXNA4/SRC/BMPR1B/SSH1/ | ROBO2/RASA |
| | | | | | | | | | | NTN4/LRIG2/SLIT2/EFNB2/RAF1/CAM | 1/LRRC4C/PT |
| | | | | | | | | | | K2G/FES/SMO/PPP3CC/PPP3R1/CAM | K2/PPP3CB/E |
| | | | | | | | | | | K2A/PLCG1 | PHA8/BMP7/S |
| | | | | | | | | | | | LIT3/ITGB1/PI |
| | | | | | | | | | | | K3CD/SHH/UN |
| | | | | | | | | | | | C5A/EFNA1/S |
| | | | | | | | | | | | SH3 |
| hsa04740 | Olfactory | 387 | −0.1840 | −1.604 | 8.90505 | 0.00058 | 0.00044 | 9052 | tags = 80%, | OR4F15/OR10G2/OR2AE1/OR4F6/OR | SLC24A4/OR1 |
| | transduction | | 87535 | 08401 | E−06 | 9514 | 8065 | | list = 47%, | 10W1/OR5H15/OR51F2/OR10K2/OR12 | 0A4/OR1E1/N |
| | | | | 9 | | | | | signal = 43% | D3/OR5M10/OR5K4/OR10Q1/OR2M7/ | CALD/OR10G3 |
| | | | | | | | | | | OR5K3/OR5B12/OR5M3/OR51B2/OR5 | |
| | | | | | | | | | | M8/OR2A12/OR4A16/OR13C3/OR5K2/ | |
| | | | | | | | | | | OR56A4/OR4D9/OR4P4/OR6S1/OR2B | |
| | | | | | | | | | | 11/OR2A5/OR3A1/OR2T1/OR10T2/OR | |
| | | | | | | | | | | 2W3/OR4K5/OR6V1/OR10G7/OR13C9/ | |
| | | | | | | | | | | OR9Q2/OR10H3/OR8H2/CALML6/OR5 | |
| | | | | | | | | | | 2N1/OR10H1/OR10A7/OR2J3/OR51A4/ | |
| | | | | | | | | | | OR1I1/OR5P2/OR11L1/OR51L1/OR6C | |
| | | | | | | | | | | 65/OR52N4/OR2AG2/OR4D6/OR6C70/ | |
| | | | | | | | | | | OR4X1/OR8B12/OR56B1/OR1N2/OR52 | |
| | | | | | | | | | | B6/OR6M1/OR2T10/OR8K3/OR8B4/OR | |
| | | | | | | | | | | 7G3/OR1L1/OR8K1/OR5H6/OR5D13/O | |
| | | | | | | | | | | R10A6/OR10A5/OR1G1/OR6Y1/OR2A2 | |
| | | | | | | | | | | /OR6X1/OR2T34/OR52M1/OR5T3/OR5 | |
| | | | | | | | | | | 1V1/OR2T8/OR11H4/OR2Y1/OR10A3/ | |
| | | | | | | | | | | OR4F5/OR8A1/OR1D2/OR1B1/OR5AC | |
| | | | | | | | | | | 2/OR2G2/OR4C46/OR10G8/OR7G1/O | |
| | | | | | | | | | | R5H14/OR8G1/OR51B4/OR8B3/OR5A | |
| | | | | | | | | | | R1/OR4C15/OR5M11/OR4K14/OR5M1/ | |
| | | | | | | | | | | OR4D11/OR13J1/OR2T27/OR1J2/OR2 | |
| | | | | | | | | | | G6/OR13F1/OR2G3/OR2A25/OR1L4/O | |
| | | | | | | | | | | R2AP1/OR52E2/OR52K2/OR1L6/OR8D | |
| | | | | | | | | | | 2/OR8J3/OR14I1/OR4L1/OR52A1/OR5 | |
| | | | | | | | | | | T1/OR10A2/OR52J3/OR51G2/OR52E4/ | |
| | | | | | | | | | | OR5L1/OR52N5/OR8J1/OR5D16/OR4D | |
| | | | | | | | | | | 2/OR7D2/OR2AK2/OR52N2/OR56B4/O | |
| | | | | | | | | | | R2A1/OR5F1/OR10R2/OR812/OR51M1/ | |
| | | | | | | | | | | OR9G1/OR1L8/OR1D4/OR5T2/OR2F2/ | |
| | | | | | | | | | | OR13C2/OR6C6/OR13D1/OR56A5/OR | |
| | | | | | | | | | | 4K13/OR6Q1/OR4K15/OR4K2/OR2S2/ | |
| | | | | | | | | | | OR8K5/OR10K1/OR5AN1/OR4C12/OR | |
| | | | | | | | | | | 10S1/OR2T35/OR2D2/OR4D10/OR1Q1 | |
| | | | | | | | | | | /OR10Z1/OR8B2/OR7G2/OR51B6/OR5 | |
| | | | | | | | | | | A2/OR52W1/OR6K3/OR52E6/OR7A5/O | |
| | | | | | | | | | | R2M3/OR4C16/OR5111/OR2B2/OR2V1/ | |
| | | | | | | | | | | OR10J1/OR4A5/OR4S2/OR13C8/OR4 | |
| | | | | | | | | | | M1/OR5AP2/OR4K1/OR10AG1/OR8D1/ | |
| | | | | | | | | | | OR51Q1/OR4S1/OR2W1/OR51F1/OR1 | |
| | | | | | | | | | | 0G4/OR51A7/OR52L1/OR1C1/OR5W2/ | |
| | | | | | | | | | | OR13C4/OR52B2/OR6C74/OR52H1/O | |
| | | | | | | | | | | R11G2/OR2T12/OR4C45/OR11H6/OR6 | |
| | | | | | | | | | | P1/OR2T6/OR14C36/OR4N4/OR2C3/O | |
| | | | | | | | | | | R6C75/OR6C1/OR5H1/OR5AU1/OR7A | |
| | | | | | | | | | | 10/OR5AS1/OR10P1/OR51A2/OR13A1/ | |
| | | | | | | | | | | OR8D4/OR6A2/OR6C4/OR2F1/OR2K2/ | |
| | | | | | | | | | | OR3A3/OR10J5/OR6K6/OR4B1/OR1A1 | |
| | | | | | | | | | | /OR51T1/OR2J2/OR5V1/OR56A3/OR7 | |
| | | | | | | | | | | E24/OR8B8/OR4E2/OR7A17/OR2AT4/ | |
| | | | | | | | | | | OR10G9/OR9K2/OR4C3/OR9G4/PRKA | |
| | | | | | | | | | | CG/OR2A14/OR4N5/OR52A5/OR6B1/O | |
| | | | | | | | | | | R2T2/OR2B3/OR2M5/RGS2/OR56A1/O | |
| | | | | | | | | | | R2T11/OR911/OR6N1/OR1K1/OR5B17/ | |
| | | | | | | | | | | OR51E1/OR4X2/OR14A16/OR6K2/OR5 | |
| | | | | | | | | | | 2I1/OR6B2/OR4A15/OR52B4/OR52D1/ | |
| | | | | | | | | | | OR10H5/OR10H4/OR1A2/OR51G1/OR | |
| | | | | | | | | | | 10H2/OR4D5/OR5C1/OR2H1/OR4K17/ | |
| | | | | | | | | | | OR7C1/OR5A1/OR52E8/OR5B21/OR2L | |
| | | | | | | | | | | 13/OR1J4/OR52K1/OR2V2/OR2H2/OR | |
| | | | | | | | | | | 9A4/OR3A2/OR1L3/OR51S1/OR12D2/ | |
| | | | | | | | | | | OR4C6/OR6C3/OR5212/CNGA3/OR5J2 | |
| | | | | | | | | | | /OR5P3/OR1E2/PDE1A/OR1N1/OR4C1 | |
| | | | | | | | | | | 3/CNGA4/OR8H1/GNG7/CALML5/OR1 | |
| | | | | | | | | | | 3G1/OR2C1/OR1S2/OR9Q1/OR5D14/O | |
| | | | | | | | | | | R8U8/SLC24A4/OR10A4/OR1E1/NCAL | |
| | | | | | | | | | | D/OR10G3 | |
| hsa04713 | Circadian | 93 | 0.3918 | 2.039 | 2.93134 | 0.00147 | 0.00112 | 4903 | tags = 55%, | CACNA1C/GRIN2C/ADCYAP1/ADCY5/ | |
| | entrainment | | 27852 | 63255 | E−05 | 6058 | 1889 | | list = 25%, | CREB1/GRIA4/GNAQ/GNAI1/PLCB1/G | |
| | | | | 7 | | | | | signal = 41% | RIN2A/RYR1/GNG4/ADCY9/GRIN2D/G | |
| | | | | | | | | | | UCY1A2/CAMK2D/FOS/PRKG2/MTNR | |
| | | | | | | | | | | 1A/ITPR1/GNG13/GNB5/GNG2/GNB3/ | |
| | | | | | | | | | | CALM3/ADCY10/PLCB4/PER2/ADCY6/ | |
| | | | | | | | | | | ADCY7/RYR3/PLCB3/GNG12/GNAS/P | |
| | | | | | | | | | | RKCG/RYR2/GNB4/GNB1/CALML3/CA | |
| | | | | | | | | | | MK2B/ADCY4/ADCY8/KCNJ5/PRKG1/ | |
| | | | | | | | | | | CAMK2G/CALM1/ADCYAP1R1/CAMK2 | |
| | | | | | | | | | | A/CACNA1D/GRIA2/PRKACA | |
| hsa04072 | Phospholipase | 140 | 0.3499 | 1.952 | 3.12157 | 0.00147 | 0.00112 | 7204 | tags = 64%, | PIP5K1C/TSC1/GRM6/ADCY5/AGPAT5 | |
| | D signaling | | 15762 | 67452 | E−05 | 6058 | 1889 | | list = 37%, | /DGKD/LPAR3/PLCB1/GRM4/GRM7/A | |
| | pathway | | | 9 | | | | | signal = 41% | DCY9/INSR/MAP2K1/JMJD7- | |
| | | | | | | | | | | PLA2G4B/DGKG/PDGFRA/SHC2/RAL | |
| | | | | | | | | | | GDS/PIK3CD/FYN/PDGFA/AGPAT1/G | |
| | | | | | | | | | | RM3/DGKH/EGFR/DGKI/PLCG2/PTGF | |
| | | | | | | | | | | R/SHC4/PLCB4/DGKZ/ADCY6/RAPGE | |
| | | | | | | | | | | F3/ADCY7/AGPAT3/PLCB3/GNAS/RH | |
| | | | | | | | | | | OA/NRAS/PLD1/AVP/GRM5/PDGFD/P | |
| | | | | | | | | | | DGFC/GNA12/ADCY4/ADCY8/SHC1/R | |
| | | | | | | | | | | HEB/SYK/AKT3/RAF1/AGPAT4/GRM2/ | |
| | | | | | | | | | | PIK3R6/PLCG1/SPHK2/GRB2/AVPR1A | |
| | | | | | | | | | | /MRAS/RRAS2/PIP5K1B/LPAR2/SPHK | |
| | | | | | | | | | | 1/AKT2/LPAR1/DNM3/GRM1/RALA/PL | |
| | | | | | | | | | | A2G4D/SOS1/CYTH2/CYTH3/FCER1A/ | |
| | | | | | | | | | | PTK2B/MAP2K2/CXCR2/ADCY2/CYTH | |
| | | | | | | | | | | 1/ADCY1/ARF1/PIK3CB/PDGFRB/ADC | |
| | | | | | | | | | | Y3/PLCB2/PLA2G4A/EGF/AVPR1B/MA | |
| | | | | | | | | | | PK1/PIK3CG | |
| hsa04015 | Rap1 | 205 | 0.3084 | 1.818 | 3.6836 | 0.00152 | 0.00115 | 7620 | tags = 62%, | RAP1GAP/ITGB2/CSF1/PARD6G/RAP | |
| | signaling | | 1663 | 21465 | E−05 | 4091 | 8396 | | list = 40%, | GEF5/FGF19/ADCY5/PRKCZ/FGF3/GN | |
| | pathway | | | 4 | | | | | signal = 38% | AQ/GNAI1/FGFR2/LPAR3/CSF1R/PLC | |
| | | | | | | | | | | B1/GRIN2A/ANGPT1/ADCY9/INSR/MA | |
| | | | | | | | | | | P2K1/ID1/CNR1/PDGFRA/SIPA1/FLT4/ | |
| | | | | | | | | | | MAPK14/FGFR4/RALGDS/MAP2K3/SIP | |
| | | | | | | | | | | A1L1/FGF8/ITGB1/PIK3CD/EFNA1/MA | |
| | | | | | | | | | | GI1/ADORA2B/PDGFA/PRKCI/TIAM1/E | |
| | | | | | | | | | | GFR/ANGPT2/PLCE1/ITGB3/PRKD3/C | |
| | | | | | | | | | | ALM3/NGF/VEGFC/PLCB4/CDH1/RAS | |
| | | | | | | | | | | GRP2/ADCY6/RAPGEF3/CTNND1/ADC | |
| | | | | | | | | | | Y7/PLCB3/DOCK4/RAP1A/GNAS/RHO | |
| | | | | | | | | | | A/PRKCG/NRAS/FGFR1/F2RL3/PDGF | |
| | | | | | | | | | | D/MAGI2/EFNA3/CALML3/PDGFC/HGF | |
| | | | | | | | | | | /ADCY4/ADCY8/SRC/RASSF5/AKT3/R | |
| | | | | | | | | | | AF1/KRIT1/MAPK12/SIPA1L2/PRKD2/T | |
| | | | | | | | | | | EK/CALM1/LCP2/PLCG1/VASP/MRAS/ | |
| | | | | | | | | | | LPAR2/ENAH/FGF18/AKT2/LPAR1/KD | |
| | | | | | | | | | | R/FGFR3/RALA/VEGFA/LAT/P2RY1/M | |
| | | | | | | | | | | AGI3/NGFR/MAP2K2/ADCY2/RAPGEF | |
| | | | | | | | | | | 2/ADCY1/GRIN1/CALM2/EPHA2/PGF/A | |
| | | | | | | | | | | RAP3/RAP1B/FGF5/PIK3CB/FGF2/PD | |
| | | | | | | | | | | GFRB/GNAI3/ADCY3/PARD3/PLCB2/E | |
| | | | | | | | | | | GF/PRKD1/MAPK1/VAV3/RAC1/PDGF | |
| | | | | | | | | | | B/ACTG1/CRK/ADORA2A/EFNA5/PIK3 | |
| | | | | | | | | | | R1/FLT1 | |
| hsa04218 | Cellular | 152 | 0.3281 | 1.861 | 7.54045 | 0.00277 | 0.00210 | 5421 | tags = 51%, | TSC1/NFATC1/GADD45A/E2F3/SMAD | |
| | senescence | | 23676 | 81754 | E−05 | 3209 | 7798 | | list = 28%, | 3/IGFBP3/MAP2K1/RAD1/MCU/LIN37/T | |
| | | | | | | | | | signal = 37% | GFB2/RB1/MAPK14/MAP2K3/NBN/TGF | |
| | | | | | | | | | | B3/PPP3CB/CCND3/PIK3CD/ITPR1/CD | |
| | | | | | | | | | | K6/CDC25A/ATM/RBBP4/NFATC4/CCN | |
| | | | | | | | | | | D2/FOXO1/BTRC/TGFBR1/CDK4/TRP | |
| | | | | | | | | | | V4/CALM3/CDKN2A/ETS1/CAPN2/CDK | |
| | | | | | | | | | | N1A/HIPK2/GATA4/NRAS/CCNA1/MYC | |
| | | | | | | | | | | /RELA/CDK2/CCNB1/RBL1/CALML3/C | |
| | | | | | | | | | | DKN2B/NFATC2/RASSF5/HIPK4/RHEB | |
| | | | | | | | | | | /CCNE1/AKT3/RAF1/FBXW11/HLA- | |
| | | | | | | | | | | E/TGFB1/MAPK12/LIN9/CALM1/GADD | |
| | | | | | | | | | | 45B/PPP3CC/PPP3R1/NFKB1/RAD50/ | |
| | | | | | | | | | | CACNA1D/HLA- | |
| | | | | | | | | | | B/MRAS/RRAS2/VDAC1/ITPR3/IL1A/F | |
| | | | | | | | | | | OXO3/PPP3CA/AKT2/SMAD2/FOXM1 | |
| hsa04022 | CGMP-PKG | 155 | 0.3193 | 1.812 | 0.00015 | 0.00426 | 0.00324 | 6998 | tags = 60%, | MYL9/CACNA1C/MYLK/NFATC1/ADCY | |
| | signaling | | 98675 | 62961 | 1721 | 6766 | 2986 | | list = 36%, | 5/CREB1/GNAQ/GNAI1/IRS1/ADRA2B/ | |
| | pathway | | | 1 | | | | | signal = 38% | KCNJ8/PDE2A/ADRA1D/PLCB1/ADCY | |
| | | | | | | | | | | 9/INSR/MAP2K1/ATP1B3/CREB5/NPP | |
| | | | | | | | | | | B/SLC8A3/GUCY1A2/ATP1A2/CNGB1/ | |
| | | | | | | | | | | PPP3CB/PRKG2/ITPR1/PRKCE/ATP2A | |
| | | | | | | | | | | 3/SLC8A1/NFATC4/ADRB3/CALM3/PL | |
| | | | | | | | | | | CB4/MYLK2/MEF2D/ADCY6/ADCY7/C | |
| | | | | | | | | | | REB3L2/GNA11/PLCB3/BAD/RHOA/GA | |
| | | | | | | | | | | TA4/ATP1B1/CALML3/ATP2B4/GNA12/ | |
| | | | | | | | | | | FXYD2/ADCY4/PDE3A/NFATC2/ADCY | |
| | | | | | | | | | | 8/KCNU1/MEF2C/PRKG1/EDNRB/ATF | |
| | | | | | | | | | | 6B/AKT3/RAF1/CALM1/PIK3R6/PPP3C | |
| | | | | | | | | | | C/PPP3R1/VASP/CACNA1D/MYH7/VD | |
| | | | | | | | | | | AC1/SRF/ITPR3/ATP2B1/SLC8A2/PPP | |
| | | | | | | | | | | 3CA/AKT2/GTF2IRD1/NPPC/ATF4/TRP | |
| | | | | | | | | | | C6/KCNMB3/ADORA3/KCNMB2/MAP2 | |
| | | | | | | | | | | K2/PPP1R12A/ADCY2/ADCY1/CALM2/ | |
| | | | | | | | | | | CREB3L3/ADRA1A/ADRA2C/GNAI3/AD | |
| | | | | | | | | | | CY3/PLCB2/SLC25A4 | |
| hsa04728 | Dopaminergic | 127 | 0.3450 | 1.888 | 0.00015 | 0.00426 | 0.00324 | 5381 | tags = 53%, | CACNA1C/ADCY5/CREB1/GRIA4/GNA | |
| | synapse | | 84503 | 18537 | 9153 | 6766 | 2986 | | list = 28%, | Q/GNAI1/PPP2R1B/PLCB1/GRIN2A/SL | |
| | | | | 8 | | | | | signal = 38% | C18A2/GNG4/DRD5/CREB5/PPP2R2C/ | |
| | | | | | | | | | | GSK3B/ARNTL/PPP2R2B/CAMK2D/GS | |
| | | | | | | | | | | K3A/FOS/MAPK14/PPP3CB/ITPR1/GN | |
| | | | | | | | | | | AL/GNG13/GNB5/ARRB1/GNG2/MAPK | |
| | | | | | | | | | | 10/GNB3/CALM3/PPP2R5C/PPP2R3A/ | |
| | | | | | | | | | | PLCB4/KIF5C/CREB3L2/PLCB3/GNG1 | |
| | | | | | | | | | | 2/CACNA1B/GNAS/PRKCG/TH/GNB4/ | |
| | | | | | | | | | | SLC18A1/GNB1/CLOCK/CALML3/CAM | |
| | | | | | | | | | | K2B/PPP2R2A/KCNJ5/ATF6B/AKT3/AR | |
| | | | | | | | | | | RB2/MAPK12/CAMK2G/CALY/CALM1/ | |
| | | | | | | | | | | PPP3CC/CAMK2A/PPP2R5E/CACNA1 | |
| | | | | | | | | | | D/GRIA2/PRKACA/DRD4/ITPR3/PPP3 | |
| | | | | | | | | | | CA/AKT2 | |
| hsa04024 | cAMP | 213 | 0.2963 | 1.755 | 0.00016 | 0.00426 | 0.00324 | 7473 | tags = 59%, | MYL9/CACNA1C/EP300/GRIN2C/NFAT | |
| | signaling | | 02405 | 29293 | 2626 | 6766 | 2986 | | list = 39%, | C1/PTGER3/ADCYAP1/ADCY5/CREB1/ | |
| | pathway | | | 3 | | | | | signal = 37% | PPARA/GRIA4/GNAI1/PDE4A/GRIN2A/ | |
| | | | | | | | | | | GIPR/DRD5/GLI3/ADCY9/CHRM1/PDE | |
| | | | | | | | | | | 4C/MAP2K1/HCN2/ATP1B3/CREB5/GR | |
| | | | | | | | | | | IN2D/POMC/CAMK2D/EDN3/CRHR2/A | |
| | | | | | | | | | | TP1A2/FOS/CREBBP/CNGB1/CHRM2/ | |
| | | | | | | | | | | GRIN3A/PIK3CD/ABCC4/ATP2A3/TIAM | |
| | | | | | | | | | | 1/MAPK10/PLCE1/GABBR2/CFTR/CAL | |
| | | | | | | | | | | M3/ADCY10/ADCY6/RAPGEF3/ADCY7/ | |
| | | | | | | | | | | CREB3L2/BAD/GLI1/RAP1A/GNAS/RH | |
| | | | | | | | | | | OA/GLP1R/PLD1/HCN4/RYR2/NPY/ED | |
| | | | | | | | | | | N2/CRH/RELA/GHSR/ATP1B1/CALML3 | |
| | | | | | | | | | | /ATP2B4/CAMK2B/FXYD2/ADCY4/PDE | |
| | | | | | | | | | | 3A/ADCY8/LHCGR/SLC9A1/PDE4D/GI | |
| | | | | | | | | | | P/OXTR/AKT3/RAF1/GABBR1/SST/CA | |
| | | | | | | | | | | MK2G/HTR1E/CALM1/ADCYAP1R1/CA | |
| | | | | | | | | | | MK2A/NFKB1/CACNA1D/GRIA2/PRKA | |
| | | | | | | | | | | CA/CRHR1/RRAS2/SSTR1/HTR1B/HHI | |
| | | | | | | | | | | P/ATP2B1/AKT2/HTR1D/ACOX1/PPP1 | |
| | | | | | | | | | | R1B/GPHA2/JUN/BDNF/MAP2K2/PPP1 | |
| | | | | | | | | | | R12A/ADCY2/SOX9/ADCY1/GRIN1/CA | |
| | | | | | | | | | | LM2/LIPE/ARAP3/RAP1B/CREB3L3/HT | |
| | | | | | | | | | | R4/PIK3CB/GNAI3/ADCY3/MAPK1/PTC | |
| | | | | | | | | | | H1/VAV3/PRKACB/RAC1/VIP/NPY1R/E | |
| | | | | | | | | | | DNRA/ADORA2A | |
| hsa04350 | TGF-beta | 92 | 0.3714 | 1.931 | 0.00016 | 0.00426 | 0.00324 | 8508 | tags = 75%, | NBL1/EP300/TFDP1/PITX2/RGMA/TGI | |
| | signaling | | 45141 | 28433 | 7577 | 6766 | 2986 | | list = 44%, | F1/TNF/SMAD3/PPP2R1B/BMP6/ID1/G | |
| | pathway | | | 5 | | | | | signal = 42% | REM1/TGFB2/ID3/CREBBP/TGFB3/BM | |
| | | | | | | | | | | P7/GDF6/ACVR1/SMAD7/TGFBR1/INH | |
| | | | | | | | | | | BC/SMURF2/ID2/LTBP1/INHBE/NODAL | |
| | | | | | | | | | | /RHOA/BMP8B/GDF7/MYC/RBL1/FBN1 | |
| | | | | | | | | | | /GREM2/SMAD9/CDKN2B/RGMB/BMP | |
| | | | | | | | | | | R1B/ACVR2A/TGFB1/INHBB/SMAD6/S | |
| | | | | | | | | | | MURF1/SMAD2/GDF5/TGIF2/E2F5/BM | |
| | | | | | | | | | | P4/BMP2/SMAD1/CUL1/BMP5/MAPK1/ | |
| | | | | | | | | | | BMP8A/ZFYVE16/ACVR2B/BMPR1A/A | |
| | | | | | | | | | | CVR1B/NEO1/ZFYVE9/E2F4/FST/MAP | |
| | | | | | | | | | | K3/AMH/THBS1/INHBA/SMAD4/ACVR1 | |
| | | | | | | | | | | C/RBX1 | |
| hsa04935 | Growth | 117 | 0.3416 | 1.842 | 0.00018 | 0.00438 | 0.00333 | 7214 | tags = 63%, | CACNA1C/EP300/ADCY5/CREB1/GNA | |
| | hormone | | 15767 | 65053 | 5666 | 9676 | 6405 | | list = 37%, | Q/GNAI1/IRS1/PLCB1/IGFBP3/ADCY9/ | |
| | synthesis, | | | 8 | | | | | signal = 40% | MAP2K1/CREB5/GHRHR/GSK3B/SHC | |
| | secretion, | | | | | | | | | 2/FOS/CREBBP/MAPK14/MAP2K3/PTK | |
| | and action | | | | | | | | | 2/PIK3CD/ITPR1/MAPK10/PLCG2/SHC | |
| | | | | | | | | | | 4/ADCY10/PLCB4/SOCS2/STAT3/ADC | |
| | | | | | | | | | | Y6/ADCY7/CREB3L2/GNA11/PLCB3/G | |
| | | | | | | | | | | NAS/PRKCG/NRAS/GHSR/ADCY4/AD | |
| | | | | | | | | | | CY8/SHC1/ATF6B/AKT3/RAF1/SST/MA | |
| | | | | | | | | | | PK12/GHR/GH2/IGFALS/PLCG1/CACN | |
| | | | | | | | | | | A1D/GRB2/PRKACA/ITPR3/SSTR1/AK | |
| | | | | | | | | | | T2/MAP3K1/STAT5B/SOS1/ATF4/SST | |
| | | | | | | | | | | R3/JAK2/MAP2K2/ADCY2/ADCY1/CRE | |
| | | | | | | | | | | B3L3/PIK3CB/GNAI3/ADCY3/PLCB2/M | |
| | | | | | | | | | | AP2K4/JUNB/MAPK1/PRKACB | |
| hsa04720 | Long-term | 64 | 0.4007 | 1.943 | 0.00025 | 0.00570 | 0.00433 | 7214 | tags = 72%, | CACNA1C/EP300/GRIN2C/GNA1/PLC | |
| | potentiation | | 63617 | 97474 | 8467 | 3502 | 4988 | | list = 37%, | B1/GRIN2A/MAP2K1/GRIN2D/CMAK2D | |
| | | | | 4 | | | | | signal = 45% | /CREBBP/PPP3CB/ITPR1/CALM3/PLC | |
| | | | | | | | | | | B4/RAPGEF3/PLCB3/RAP1A/RPS6KA2 | |
| | | | | | | | | | | /PRKCG/NRAS/GRM5/CALML3/CAMK2 | |
| | | | | | | | | | | B/ADCY8/RAF1/CAMK2G/CALM1/PPP | |
| | | | | | | | | | | 3CC/PPP3R1/CAMK2A/GRIA2/PRKAC | |
| | | | | | | | | | | A/ITPR3/PPP3CA/RPS6KA1/GRM1/AT | |
| | | | | | | | | | | F4/MAP2K2/ADCY1/GRIN1/CALM2/RA | |
| | | | | | | | | | | P1B/PPP1R1A/PLCB2/MAPK1/PRKAC | |
| | | | | | | | | | | B | |
| hsa04934 | Cushing | 154 | 0.3090 | 1.755 | 0.00030 | 0.00628 | 0.00477 | 5130 | tags = 47%, | CACNA1C/FZD2/WNT5A/WNT1/WNT8 | |
| | syndrome | | 04449 | 0.1298 | 3599 | 0701 | 3693 | | list = 27%, | B/WNT2/E2F3/ADCY5/CREB1/GNAQ/G | |
| | | | | 5 | | | | | signal = 35% | NAI1/PLCB1/CYP11A1/ARMC5/WNT10 | |
| | | | | | | | | | | B/TCF7L2/ADCY9/MAP2K1/CREB5/GS | |
| | | | | | | | | | | K3B/POMC/CAMK2D/RB1/CRHR2/ITP | |
| | | | | | | | | | | R1/CDK6/FZD8/EGFR/PBX1/WNT2B/C | |
| | | | | | | | | | | DK4/CDKN2A/PLCB4/ASH2L/ADCY6/L | |
| | | | | | | | | | | EF1/ADCY7/CREB3L2/GNA11/NCEH1/ | |
| | | | | | | | | | | PLCB3/CDKN1A/NR4A1/RAP1A/GNAS/ | |
| | | | | | | | | | | WNT9A/CRH/PDE11A/CDK2/WNT10A/ | |
| | | | | | | | | | | AIP/WNT6/KCNA4/CAMK2B/APC/CYP2 | |
| | | | | | | | | | | 1A2/ADCY4/WNT5B/CDKN2B/ADCY8/F | |
| | | | | | | | | | | ZD7/ATF6B/WNT11/CCNE1/ARNT/CA | |
| | | | | | | | | | | MK2G/CAMK2A/CACNA1D/PRKACA/C | |
| | | | | | | | | | | RHR1/KCNK2/DVL1/ITPR3 | |
| hsa05320 | Autoimmune | 46 | −0.3679 | −2.070 | 0.00032 | 0.00628 | 0.00478 | 4449 | tags = 47%, | HLA-C/HLA-DMA/CD86/CD80/HLA- | |
| | thyroid | | 95657 | 71073 | 3003 | 9057 | 0044 | | list = 23%, | DQB1/HLA-G/IL10/IL4/HLA- | |
| | disease | | | 2 | | | | | signal = 36% | DPB1/CD28/HLA-DOB/IFNA8/HLA- | |
| | | | | | | | | | | DRB5/PRF1/FASLG/HLA- | |
| | | | | | | | | | | DOA/IFNA10/HLA- | |
| | | | | | | | | | | F/TG/TSHR/TPO/HLA-A | |
| hsa04550 | Signaling | 138 | 0.3271 | 1818 | 0.00042 | 0.00789 | 0.00600 | 4840 | tags = 46%, | ISL1/FZD2/WNT5A/WNT1/WNT8B/WN | |
| | pathways | | 66435 | 60711 | 9407 | 6318 | 1654 | | list = 25%, | T2/FGFR2/SMAD3/TOX1/JAK1/WNT10 | |
| | regulating | | | 2 | | | | | signal = 34% | B/POU5F1/MAP2K1/GSK3B/ID1/ID3/NE | |
| | pluripotency | | | | | | | | | UROG1/MAPK14/FGFR4/ZFHX3/PCGF | |
| | of stem cells | | | | | | | | | 6/PIK3CD/REST/DLX5/FZD8/ACVR1/K | |
| | | | | | | | | | | AT6A/TCF3/HAND1/INHBC/ID2/WNT2B | |
| | | | | | | | | | | /STAT3/INHBE/NODAL/SOX2/PAX6/NR | |
| | | | | | | | | | | AS/FGFR1/MYC/WNT9A/LHX5/ONECU | |
| | | | | | | | | | | T1/WNT10A/WNT6/APC/SMAD9/WNT5 | |
| | | | | | | | | | | B/BMPR1B/RIF1/IL6ST/FZD7/ACVR2A/ | |
| | | | | | | | | | | WNT11/ESRRB/AKT3/RAF1/MAPK12/H | |
| | | | | | | | | | | OXD1/INHBB/JAK3/GRB2/SMARCAD1 | |
| hsa04921 | Oxytocin | 149 | 0.3077 | 1.742 | 0.00051 | 0.00857 | 0.00651 | 7465 | tags = 64%, | MYL9/CACNA1C/MYLK/NFATC1/ADCY | |
| | signaling | | 16772 | 50867 | 2157 | 477 | 7316 | | list = 39%, | 5/PRKAG1/GNA1/GNAI1/PLCV1/RYR1 | |
| | pathway | | | 7 | | | | | signal = 39% | /ADCY9/MYL6/MAP2K1/JMJD7- | |
| | | | | | | | | | | PLA2G4B/GUCY1A2/CAMK2D/FOS/CA | |
| | | | | | | | | | | MK1/CAMK1D/PPP3CB/ITPR1/CD38/C | |
| | | | | | | | | | | ACNG8/EGFR/NFATC4/CACNA2D2/CA | |
| | | | | | | | | | | LM3/PLCB4/MYLK2/ADCY6/ADCY7/CA | |
| | | | | | | | | | | CNG4/RYR3/PLCB3/EEF2K/CDKN1A/G | |
| | | | | | | | | | | NAS/RHOA/PRKCG/NRAS/RYR2/KCNJ | |
| | | | | | | | | | | 4/CALML3/CAMK2B/PPP1R12C/ADCY | |
| | | | | | | | | | | 4/NFATC2/ADCY8/SRC/KCNJ5/MEF2C | |
| | | | | | | | | | | /PTGS2/OXTR/RAF1/CAMK2G/MAPK7/ | |
| | | | | | | | | | | CALM1/PIK3R6/CACNA2D3/KCNJ14/P | |
| | | | | | | | | | | PP3CC/PPP3R1/CAMK2A/CACNA1D/P | |
| | | | | | | | | | | RKACA/ITPR3/MAP2K5/PPP3CA/CAC | |
| | | | | | | | | | | NG6/CACNB1/RCAN1/PLA2G4D/KCNJ | |
| | | | | | | | | | | 3/CACNA2D1/JUN/PRKAA1/MAP2K2/P | |
| | | | | | | | | | | PP1R12A/ADCY2/ADCY1/CACNG7/CA | |
| | | | | | | | | | | LM2/CAMKK2/GNAI3/ADCY3/PLCB2/P | |
| | | | | | | | | | | LA2G4A/CACNG1/MAPK1/CACNG3/PI | |
| | | | | | | | | | | K3CG/PRKACB/ACTG1/CACNG5/MYL | |
| | | | | | | | | | | K3 | |
| hsa04926 | Relaxin | 125 | 0.3252 | 1.775 | 0.00051 | 0.00857 | 0.00651 | 7124 | tags = 61%, | RLN1/ADCY5/CREB1/PRKCZ/GNAI1/P | |
| | signaling | | 89391 | 06485 | 8113 | 477 | 7316 | | list = 37%, | LCB1/COL4A3/GNG4/ADCY9/MAP2K1/ | |
| | pathway | | | 5 | | | | | signal = 38% | CREB5/NOS2/SHC2/FOS/MAPK14/PIK | |
| | | | | | | | | | | 3CD/GNG13/GNB5/ARRB1/GNG2/EGF | |
| | | | | | | | | | | R/MAPK10/GNB3/TGFBR1/COL4A1/SH | |
| | | | | | | | | | | C4/VEGFC/PLCB4/ADCY6/ADCY7/CR | |
| | | | | | | | | | | EB3L2/PLCB3/GNG12/MMP2/GNAS/IN | |
| | | | | | | | | | | SL3/NRAS/GNB4/RELA/GNB1/ADCY4/ | |
| | | | | | | | | | | ADCY8/SRC/SHC1/EDNRB/ATF6B/AK | |
| | | | | | | | | | | T3/RAF1/ARRB2/TGFB1/MAPK12/NFK | |
| | | | | | | | | | | B1/GRB2/PRKACA/AKT2/SMAD2/VEG | |
| | | | | | | | | | | FA/SOS1/ATF4/RXFP3/ACTA2/JUN/CO | |
| | | | | | | | | | | L3A1/MMP9/MAP2K2/ADCY2/ADCY1/C | |
| | | | | | | | | | | REB3L3/RXFP4/PIK3CB/GNAI3/ADCY3 | |
| | | | | | | | | | | /PLCB2/MAP2K4/MAPK1/PRKACB | |

AI prediction of AD. A total of 262,046 intragenic (CpGs within gene region) and 94,750 extragenic (CpGs outside of gene region) CpG sites were used for unbiased AI analysis. Training algorithms were developed using 15 AD cases and 13 controls and the performance of these algorithms was independently validated in a separate test group (10 AD cases and 10 controls).

The performance of the 20 intragenic CpG algorithms in the test group, when a bootstrapping approach was used, achieved excellent diagnostic performance in the test group AUC for the AI platforms (0.949-0.999). For example, in the test group, DL achieved an AUC (95% CI)=0.998 (0.950-1.0), with 94.5% sensitivity and specificity respectively. The performance was close to that of the training data used to develop the algorithms. Similarly, excellent diagnostic performance was achieved in the independent test group using a 20 CpG intragenic algorithm-based 10-fold cross-validation. The AUCs=0.939-0.984 for the test group. For example, DL achieved an AUC (95% CI)=0.984 (0.92-1.0), with 92f.5% sensitivity and 93.5% specificity.

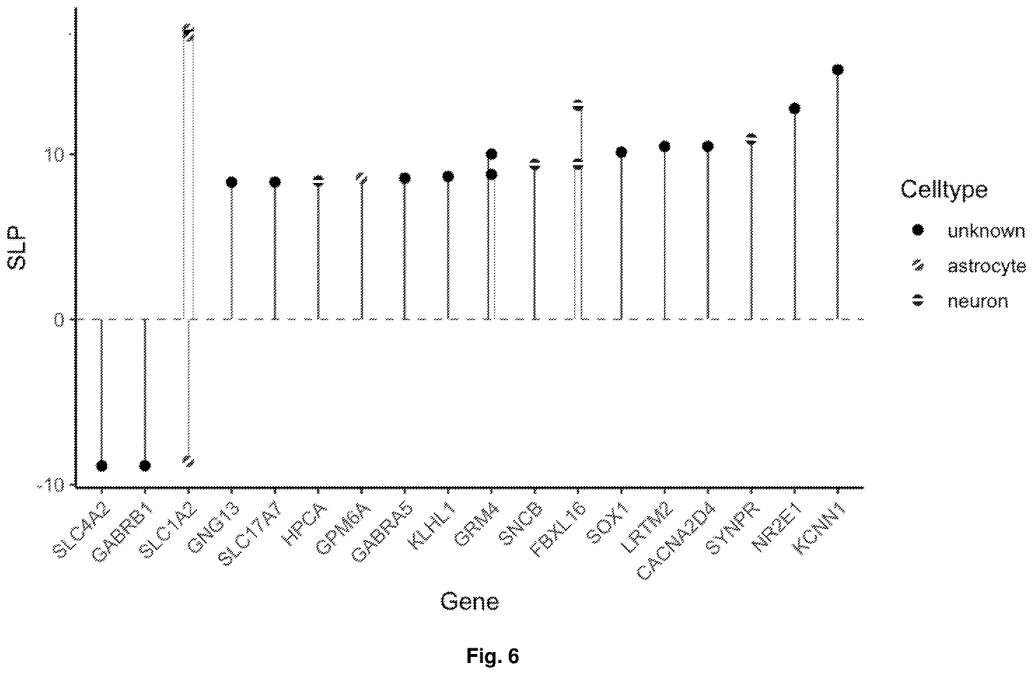

The study was focused on circulating cf DNA and therefore gene expression was not evaluated. However, the possibility of a correlation between circulating cf DNA methylation analysis and previously published brain transcriptomic studies was investigated. O'Connell et al. (2020)²⁵ collated and performed bioinformatic analysis of published studies that evaluated mRNA expression data. A total of 12,000 human specimens evaluated 17,000 protein-coding genes and their feasibility as blood biomarkers for neurological damage. Genes were considered and ranked as possible biomarkers for brain injury based on the following criteria: (i) enrichment in brain tissue compared to non-neuronal tissue, (ii) abundantly expressed in the brain, and (iii) low expression variability across various brain regions. Of the top 100 “brain biomarker” genes identified by O'Connell et al. (2020)²⁵, the study reports 16 genes that were differentially methylated (adjusted p<0.05). They include, C11orf87, FBXL16, GABRA5, GNG13, GPM6A, GRM4, HPCA, KCNN1, KLHL1, LRTM2, NR2E1, SLC17A7, SLC1A2, SNCB, SOX1 and SYNPR. The primary neurological cell type of preferential expression of these is shown in FIG. 6.

Discussion Circulating cf DNA is classically released into the bloodstream from damaged or dead tissues into the brain 26. Using DNA-methylation analysis of circulating cf DNA, extensive epigenetic modification in cytosine nucleotides in genes from people suffering from AD as compared to cognitively healthy control subjects was found. Multiple different algorithms were evaluated using different AI platforms and different analytic approaches. Using AI analysis with DNA methylation from data to include both intra- and extra-genic CpG markers, diagnose AD was diagnosed with excellent accuracy. The observed diagnostic accuracy was sustained using different analytic approaches (e.g., cross-validation and bootstrapping) An important objective of our study was to use cf DNA to further elucidate the molecular mechanisms of AD. Epigenetic changes in molecular pathways previously linked to neurological disease were identified, and thus are readily reconcilable with our current understanding of AD.

Increased hypermethylation of CpGs in cf DNA from AD sufferers across the genome as compared to controls was found (FIG. 2C). The gene promoter and 5′UTR regions were increasingly hypermethylated as opposed to hypomethylated in AD. Hypermethylation classically regulates the genome by silencing gene promoters, silencing or at least downregulates (partial activity) the enhancers, and controlling non-coding RNA genes.²⁷ Overall, these results suggest the possible downregulation of gene expression in association with AD.

Some of the genes that were found to be significantly differentially methylated and their known or putative roles in neuronal function and AD were reviewed. KDM2A was the significantly differentially methylated (hyper-methylated) gene at the Transcription Start Site 1500 (TSS1500; adjusted p=7.45×10⁻⁰⁵) and is involved in histone demethylase activity. Essentially, it recruits HP1 and establishes H3K9 and CpG methylation to form mature heterochromatin and regulates complex nucleosome binding mechanisms. Disrupted nucleosome binding results in transcriptional deregulation and genomic instability.²⁸ This mechanism was reported to be disrupted in synaptic genes of ADaffected brains.^{29, 30} The second most significantly differentially methylated gene was ZNF529 which was hyper-methylated at TSS1500 and 5′ UTR (adjusted p=7.45×10⁻⁰⁵). While this gene has not previously been reported to be associated with neurodegenerative disorders, blocking its activity resulted in increased low-density lipoprotein (LDL) receptor expression and increased cholesterol (LDL-c) uptake by cells in association with cardiovascular diseases (CVD)³¹. It is notable that, CVD and LDL-c both are significant AD risk modifiers³². The next gene found to undergo significant methylation change was HOXD13. This gene was hyper-methylated on exon 1 and is involved in regulating neuronal stemness.³³ The role of this gene in AD pathogenesis is yet to be explored.

AI algorithms are increasingly being utilized to build accurate disease predictors based on big data from omics experiments³⁴. Excellent AD diagnostic models using multiple platforms (DL, SVM, GLM, PAM, and RF) that were validated in an independent test group were developed. The AI algorithms rank the contribution of markers. Based on AI ranking, CpG markers that appeared to be the best individual AD predictors across the different platforms were identified. These CpGs are: cg19760734 (TACC1), cg05876416 (FAM173B), cg00234736 (ELMO1), cg21243612 (C9orf6), cg24040188 (RBBP8). They consistently appeared among the four AI algorithms (SVM, PAM, RF, and DL) for AD diagnosis. The literature was reviewed to determine the potential biological relevance of these genes to AD. TACC1, FAM173B, C9orf6, and RBBP8 are expressed in various regions of the brain according to “The Genotype-Tissue Expression (GTEx)” portal³⁵. ELMO1 has been linked to AD. Knock-down of ELMO1 inhibits neurite outgrowth and deactivates Rac1 and Rac1-mediated neurite outgrowth leading to age-dependent neurodegeneration and AD development.^{36, 37}

Disease and functional enrichment: Beyond the possible role of individual genes, gene networks were evaluated to further our understanding of AD. Significant over-representation of gene pathways linked to neurological disease was found, for example, the Calcium signaling pathway, Glutamatergic synapse, Hedgehog signaling pathway, Axon guidance, and Olfactory transduction.

Calcium signaling pathway: Calcium is an important signaling ion that regulates important deficits in AD. Calcium signaling is linked to Calcium/calmodulin-dependent kinases, MAPK/ERKs, and the CREB cycle which regulates homeostasis in AD³⁸⁻⁴⁰. In AD, the amyloidogenic pathway remodels neuronal Ca²⁺ signaling leading to enhanced cellular entry of Ca²⁺ through ryanodine receptors⁴¹. Disrupted cellular calcium can induce synaptic deficits that promote the accumulation of amyloid plaques (Aβ) and neurofibrillary tangles,⁴² marquee pathological features of AD. The gene CACNA1C displayed altered methylation in 5 CpG loci (3 hyper- and 2 hypo-methylated). The interaction between RYR3 and CACNA1C is crucial in terms of AD pathogenesis. Both genes are involved in modulating Aβ load and increasing intracellular calcium levels.⁴³ MYLK (hypermethylated CpGs in AD as reported herein) codes for myosin light chain kinase (MLCK). MLCK is involved in hippocampal neuronal microfilament damage in hyperglycemia. Chronic hyperglycemia induces irregularities in nuclear shape, induces shrinking of synapses, and thus damages the neuronal microfilament.⁴⁴ Hyperglycemia is an established risk factor for AD development⁴⁴.

Glutamatergic synapse: Excitatory glutamatergic neurotransmission is essential for synaptic plasticity and neuronal survival. This type of neurotransmission occurs via the N-methyl-d-aspartate receptor (NMDAR).⁴⁵ Synaptic NMDAR supports plasticity and promotes cell survival while extrasynaptic NMDAR promotes excitotoxicity which leads to cell death and neurodegeneration, a hallmark of AD.⁴⁵ Differentially methylated genes involved in Glutamatergic synapse include the PPP3CB gene. PPP3CB codes for protein phosphatases that reverse the activity of protein kinases which are important in the process of tau and amyloid-β accumulation.⁴⁶ PPP3CB was previously reported to be linked to long-term memory potentiation in AD.⁴⁷ Epigenetic changes in genes from the solute carrier (SLC) superfamily of solute carrier transporters were identified. The SLC superfamily participates in the uptake of small molecules into cells⁴⁸. 86 differentially methylated SLC superfamily genes in the study were identified; 5 of which (SLC8A3, SLC1A2, SLC1A6, SLC17A7, and SLC24A4) were identified to be enriched in significant signaling pathways in this study. SLC8A3 is involved in calcium signaling, and along with SLC1A2, SLC1A6, and SLC17A7 are known to participate in glutamatergic synapse, while SLC24A4 is involved in Olfactory transduction. In the brain, SLC family transporters are important for returning synaptic neurotransmitters to the presynaptic neurons.^{48, 49} Altered expression of these genes can lead to synaptic dysfunction, an important feature of AD pathogenesis.⁵⁰

Hedgehog signaling pathway: The Sonic hedgehog (SHH) signaling pathway is involved in neurogenesis, neural patterning, and cell survival during nervous system development^{51, 52}. SHH signaling requires intact primary cilia in brain cells and fails with structurally disrupted cilia. Elevated Aβ peptide levels that result in plaque formation disrupt the cilial structure and thus inhibit SHH signaling. Human ciliary disease results in cognitive impairment, a feature of AD.⁵² Epigenetic changes in genes involved in the SHH signaling pathway were found. The CDON gene may participate in the generation of neurons and in nervous system development.⁵³ The CUL3 gene is one of the ubiquitin ligase genes and it was found to be downregulated in various brain regions in AD subjects.⁵⁴ Hypermethylation of this gene is reported, which is consistent with the downregulation of gene expression. GLI3 is a gene that was found to be hypermethylated and has previously been linked to language dysfunction in AD.⁵⁵

Axon guidance: Axonal guidance is a neurodevelopmental process in which the axons are directed to their target neurons. The molecules involved in axon guidance have also been found to play a key role in immune and inflammatory responses in the nervous system⁵⁶. Several of the genes involved in axon guidance were also found to be differentially methylated in the study. BMP7 is involved in Axon guidance⁵⁷ and in the recovery of cardiac function after myocardial infarction⁵⁸. Hypomethylation of this gene in AD was found. BMP7 is a candidate gene for vascular diseases⁵⁹. The gene variants of BMP7 stimulate inflammation and are associated with acute myocardial infarction and AD⁵⁰. The other gene identified in axon guidance is MYL9, which codes for the myosin light chain. Biologically, it interacts with NMDAR which regulates synaptic plasticity and thereby regulates neurons in the hippocampus.^{61, 62} SEMA6D is a cardiac-expressed gene that codes for semaphorins. SEMA6D interacts with TREM2, which is a gene that is involved in axonal growth in AD and has been linked to AD pathogenesis.⁶³

Olfactory transduction: The olfactory neurons are thought to provide an entry portal into the brain for external substances believed to be involved in the pathophysiology of major neurodegenerative disorders such as AD and Parkinson's disease. Diminution of the sense of smell is a common feature of early-stage Parkinson's disease.⁶⁴ NCALD codes for Neurocalcin delta, which is a neuronal calcium sensor.⁶⁵ Complete loss of function of the gene is believed to impair neurogenesis, and reduced expression in the brains of AD subjects has been reported.^{66, 67}

As brain cells also contribute to the circulating cf DNA pool, the possible correlation between the findings of methylation of this study and published brain transcriptomic studies was investigated. Of the top 100 ‘biomarker’ genes indicating neurological damage identified by O'Connell et al. (2020)²⁵; 16 of these damage genes which are known to be differentially expressed in the brain are also differentially methylated (adjusted p<0.05) in cf DNA from AD sufferers. Further, based on specific biomarker enrichment analysis, astrocytes, and neuronal coding genes were found to be significantly differentially methylated along with other genes in which the cell type and gene is preferentially expressed are unknown (Supplemental FIG. 3). The differentially methylated astrocyte coding genes found to be enriched in AD cases were, SLC1A2 (one CpG hypomethylated and two hypermethylated) and GPM6A (1 CpG hypermethylated). The differentially methylated neuron enriched genes were, FBXL16, HPCA, SNCB, and SYNPR. All of these neuronal-associated CpGs were hypermethylated in this study. For the remaining 12 differentially methylated genes, the origin of the brain cells in which they are differentially expressed is listed as “currently unknown”.²⁵ Overall, these findings suggest a possible correlation between gene expression in the brain and the circulating cf DNA methylation markers.

Although in this study a relatively modest sample size was used, the power of using cf DNA epigenetics markers as a diagnostic tool for AD was demonstrated.

Conclusion. Significant genome-wide methylation changes in circulating cf DNA from AD subjects are reported. Using multiple AI techniques and either intragenic or extragenic CpG markers in an independent test and validation group, an excellent diagnostic accuracy (AUCs of ≥0.9) for AD is found using CpG methylation analysis based on circulating cf DNA. Intriguing and plausible pathogenic information on AD development was also generated. Multiple genes that were epigenetically altered in AD in the study were previously known or linked to the control of synaptic activity, neuronal stemness, and age-dependent neurodegeneration. A substantial number of genes that are highly ranked as plausible markers for brain damage based on their differential expression in the brain were found to be differentially methylated in circulating cf DNA. Finally, using pathway analysis, epigenetic dysregulation of gene networks involved in neurotransmission, synaptic plasticity, cell survival, learning, and function of memory was found.

### Example 2

AI is a powerful tool for discrimination and group classification. It is able to combine a large number of features or predictors to achieve this classification which when combined improves the ability to distinguish one group from another. This capability to a large degree explains the superiority of AI over conventional statistical analysis. The latter employs a small number of features in an attempt to achieve prediction and group discrimination. Using AI, it was observed that as the number of features and predictors simultaneously employed increased, the accuracy of discrimination (represented commonly by the area under the ROC curve, sensitivity, and specificity) also increased. As a consequence, 100 CpG marker prediction algorithms were developed for each AI platform for the prediction of Alzheimer's Disease. Starting from >200,000 intragenic CpGs and >200,000 extragenic CpGs that met quality standards for methylation assays, a group of 6 separate AI algorithms for the prediction of AD based on intragenic or extragenic CpGs was developed.

Each set of AI predictive algorithms was first developed in a group of cases and unaffected controls called the ‘training’ group. Once the algorithm (100 CpG markers per AI platform) was developed in the training group it was subsequently tested in the independent group of AD cases and controls call the ‘test” group. This maneuver was used to confirm the performance of the algorithm and provide independent validation of its accuracy in a separate population.

Table 1A lists the performances of intragenic markers (algorithms) for AD detection for each of the panel of 6 AI platforms in the training data set used to develop the predictive algorithms. The performance of these same CpG markers that were then deployed in the independent test group is shown in Table 1B. Tables 1A and 1B use the cross-validation (CV) statistical approach for AD prediction using the intragenic CpG markers.

Tables 2A and 2B use the Bootstrapping approach for AD prediction using the extragenic CpG markers. Table 2A shows the performance of the algorithms in the development or training group. Table 2B shows the performance of the same algorithms (same extragenic CpGs) in an independent or test group.

Tables 3A and 3B evaluate the extragenic CpG markers using the cross-validation (CV) statistical technique. Table 3A shows the performance of the algorithms in the development or training group. Table 3B shows the performance of the same AI algorithms (same extragenic CpG markers) in an independent test group.

Tables 4A and 4B evaluate the performance of extragenic markers using the Bootstrapping statistical approach. Table 4A shows the performance of the 6 different AI algorithms (each using 100 CpGs) for the detection of AD in a training or development group. Table 4B shows the performance of the same algorithms (same CpG markers) in the independent test group.

For each of the AI platforms using intragenic CpG markers, there is extensive overlap between CpGs used in the different AI algorithms. The same applies to the extragenic CpGs. Table 5 (Intragenic markers and genes-consolidated list) is a consolidated list of all the separate intragenic CpGs (and associated genes) that have been used in the different AI algorithms.

Similarly, Table 6 (Extragenic markers-consolidated list) lists all the independent extragenic CpG markers used in the 6 different AI algorithms for AD prediction and for which we are laying claims.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

All publications, patents, and patent applications cited in this specification are incorporated herein by reference in their entirety as if each individual publication, patent, or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

1. Hampel H, Toschi N, Baldacci F, Zetterberg H, Blennow K, Kilimann I, et al. Alzheimer's disease biomarker-guided diagnostic workflow using the added value of six combined cerebrospinal fluid candidates: Abeta1-42, total-tau, phosphorylated-tau, NFL, neurogranin, and YKL-40. Alzheimers Dement. 2018; 14 (4): 492-501.
2. Winblad B, Amouyel P, Andrieu S, Ballard C, Brayne C, Brodaty H, et al. Defeating Alzheimer's disease and other dementias: a priority for European science and society. Lancet Neurol. 2016: 15 (5): 455-532.
3. Handy D E, Castro R, Loscalzo J. Epigenetic modifications: basic mechanisms and role in cardiovascular disease. Circulation. 2011; 123 (19): 2145-56.
4. Kurdyukov S, Bullock M. DNA Methylation Analysis: Choosing the Right Method. Biology (Basel). 2016; 5 (1).
5. Esposito M, Sherr G L. Epigenetic Modifications in Alzheimer's Neuropathology and Therapeutics. Front Neurosci. 2019; 13:476.
6. Finotti A, Allegretti M, Gasparello J, Giacomini P, Spandidos D A, Spoto G, et al. Liquid biopsy and PCR-free ultrasensitive detection systems in oncology (Review). Int J Oncol. 2018; 53 (4): 1395-434.
7. Tadimety A, Closson A, Li C, Yi S, Shen T, Zhang J X J. Advances in liquid biopsy on-chip for cancer management: Technologies, biomarkers, and clinical analysis. Crit Rev Clin Lab Sci. 2018; 55 (3): 140-62.
8. Liu Q, Ma J, Deng H, Huang S J, Rao J, Xu W B, et al. Cardiac-specific methylation patterns of circulating DNA for identification of cardiomyocyte death. BMC cardiovascular disorders. 2020; 20 (1): 310.
9. Bronkhorst A J, Ungerer V, Diehl F, Anker P, Dor Y, Fleischhacker M, et al. Towards systematic nomenclature for cell-free DNA. Human Genetics. 2021; 140 (4): 565-78.
10. Garg N, Hidalgo L G, Aziz F, Parajuli S, Mohamed M, Mandelbrot D A, et al., editors. Use of Donor-Derived Cell-Free DNA for Assessment of Allograft Injury in Kidney Transplant Recipients During the Time of the Coronavirus Disease 2019 Pandemic. Transplantation Proceedings; 2020: Elsevier.
11. Knight S R, Thorne A, Faro M L L. Donor-specific cell-free DNA as a biomarker in solid organ transplantation. A systematic review. Transplantation. 2019; 103 (2): 273-83.
12. Pai M C, Kuo Y M, Wang I F, Chiang P M, Tsai K J. The Role of Methylated Circulating Nucleic Acids as a Potential Biomarker in Alzheimer's Disease. Mol Neurobiol. 2019; 56 (4): 2440-9.
13. Weinstein G, Seshadri S. Circulating biomarkers that predict incident dementia. Alzheimers Res Ther. 2014; 6 (1): 6.
14. Hampel H, Goetzl E J, Kapogiannis D, Lista S, Vergallo A. Biomarker-Drug and Liquid Biopsy Co-development for Disease Staging and Targeted Therapy: Cornerstones for Alzheimer's Precision Medicine and Pharmacology. Front Pharmacol. 2019; 10:310.
15. Bahado-Singh R O, Sonek J, Mckenna D, Cool D, Aydas B, Turkoglu O, et al. Artificial Intelligence and amniotic fluid multiomics analysis: The prediction of perinatal outcome in asymptomatic short cervix. Ultrasound Obstet Gynecol. 2018.
16. Bahado-Singh R O, Yilmaz A, Bisgin H, Turkoglu O, Kumar P, Sherman E, et al. Artificial intelligence and the analysis of multi-platform metabolomics data for the detection of intrauterine growth restriction. PLOS One. 2019; 14 (4):e0214121.
17. Alpay Savasan Z, Yilmaz A, Ugur Z, Aydas B, Bahado-Singh R O, Graham S F. Metabolomic Profiling of Cerebral Palsy Brain Tissue Reveals Novel Central Biomarkers and Biochemical Pathways Associated with the Disease: A Pilot Study. 2019; 9 (2).
18. Bahado-Singh R O, Vishweswaraiah S, Aydas B, Mishra N K, Guda C, Radhakrishna U. Deep Learning/Artificial Intelligence and Blood-Based DNA Epigenomic Prediction of Cerebral Palsy. International Journal of Molecular Sciences. 2019; 20 (9): 2075.
19. McKhann G M, Knopman D S, Chertkow H, Hyman B T, Jack C R, Jr., Kawas C H, et al. The diagnosis of dementia due to Alzheimer's disease: recommendations from the National Institute on Aging-Alzheimer's Association workgroups on diagnostic guidelines for Alzheimer's disease. Alzheimers Dement. 2011; 7 (3): 263-9.
20. Bartak B K, Kalmar A, Galamb O, Wichmann B, Nagy Z B, Tulassay Z, et al. Blood Collection and Cell-Free DNA Isolation Methods Influence the Sensitivity of Liquid Biopsy Analysis for Colorectal Cancer Detection. Pathol Oncol Res. 2019; 25 (3): 915-23.
21. Sheinerman K S, Toledo J B, Tsivinsky V G, Irwin D, Grossman M, Weintraub D, et al. Circulating brain-enriched microRNAs as novel biomarkers for detection and differentiation of neurodegenerative diseases. Alzheimers Res Ther. 2017; 9 (1): 89.
22. Hardy T, Zeybel M, Day C P, Dipper C, Masson S, McPherson S, et al. Plasma DNA methylation: a potential biomarker for stratification of liver fibrosis in non-alcoholic fatty liver disease. Gut. 2017; 66 (7): 1321-8.
23. Ramirez K, Fernández R, Collet S, Kiyar M, Delgado-Zayas E, Gómez-Gil E, et al. Epigenetics Is Implicated in the Basis of Gender Incongruence: An Epigenome-Wide Association Analysis. Front Neurosci. 2021; 15.
24. Alakwaa F M, Chaudhary K, Garmire L X. Deep Learning Accurately Predicts Estrogen Receptor Status in Breast Cancer Metabolomics Data. J Proteome Res. 2018; 17 (1): 337-47.
25. O'Connell G C, Alder M L. Large-scale informatic analysis to algorithmically identify blood biomarkers of neurological damage. 2020; 117 (34): 20764-75.
26. Kustanovich A, Schwartz R, Peretz T, Grinshpun A. Life and death of circulating cell-free DNA. Cancer Biol Ther. 2019; 20 (8): 1057-67.
27. Ehrlich M. DNA hypermethylation in disease: mechanisms and clinical relevance. Epigenetics. 2019; 14 (12): 1141-63.
28. Borgel J, Tyl M, Schiller K, Pusztai Z, Dooley C M, Deng W, et al. KDM2A integrates DNA and histone modification signals through a CXXC/PHD module and direct interaction with HP1. Nucleic acids research. 2017; 45 (3): 1114-29.
29. Mastroeni D, Delvaux E, Nolz J, Tan Y, Grover A, Oddo S, et al. Aberrant intracellular localization of H3k4me3 demonstrates an early epigenetic phenomenon in Alzheimer's disease. Neurobiol Aging. 2015; 36 (12): 3121-9.
30. Park S Y, Seo J, Chun Y S. Targeted Downregulation of kdm4a Ameliorates Tau-engendered Defects in Drosophila melanogaster. J Korean Med Sci. 2019; 34 (33): e225-e.
31. Nielsen J B, Rom O, Surakka I, Graham S E. Loss-of-function genomic variants highlight potential therapeutic targets for cardiovascular disease. 2020; 11 (1): 6417.
32. Zhou Z, Liang Y, Zhang X, Xu J, Lin J, Zhang R, et al. Low-Density Lipoprotein Cholesterol and Alzheimer's Disease: A Systematic Review and Meta-Analysis. Frontiers in Aging Neuroscience. 2020; 12.
33. Konar A, Kalra R S, Chaudhary A, Nayak A, Guruprasad K P, Satyamoorthy K, et al. Identification of Caffeic Acid Phenethyl Ester (CAPE) as a Potent Neurodifferentiating Natural Compound That Improves Cognitive and Physiological Functions in Animal Models of Neurodegenerative Diseases. Frontiers in aging neuroscience. 2020; 12:561925-.
34. Asada K, Kaneko S, Takasawa K, Machino H, Takahashi S, Shinkai N, et al. Integrated Analysis of Whole Genome and Epigenome Data Using Machine Learning Technology: Toward the Establishment of Precision Oncology. Frontiers in Oncology. 2021; 11.
35. Consortium G T. The Genotype-Tissue Expression (GTEx) project. Nature genetics. 2013; 45 (6): 580-5.
36. Li W, Tam K M V, Chan W W R, Koon A C, Ngo J C K, Chan H Y E, et al. Neuronal adaptor FE65 stimulates Rac1-mediated neurite outgrowth by recruiting and activating ELMO1. J Biol Chem. 2018; 293 (20): 7674-88.
37. Kikuchi M, Sekiya M, Hara N, Miyashita A, Kuwano R, Ikeuchi T, et al. Disruption of a RAC1-centred network is associated with Alzheimer's disease pathology and causes age-dependent neurodegeneration. Hum Mol Genet. 2020; 29 (5): 817-33.
38. Ghosh A, Giese K P. Calcium/calmodulin-dependent kinase Il and Alzheimer's disease. Molecular Brain. 2015; 8 (1): 78.
39. Zhu X, Lee H G, Raina A K, Perry G, Smith M A. The role of mitogen-activated protein kinase pathways in Alzheimer's disease. Neuro-Signals. 2002; 11 (5): 270-81.
40. Saura C A, Valero J. The role of CREB signaling in Alzheimer's disease and other cognitive disorders. Reviews in the neurosciences. 2011; 22 (2): 153-69.
41. Berridge M J. Calcium signalling and Alzheimer's disease. Neurochemical research. 2011; 36 (7): 1149-56.
42. Tong B C-K, Wu A J, Li M, Cheung K-H. Calcium signaling in Alzheimer's disease & therapies. Biochimica et Biophysica Acta (BBA)-Molecular Cell Research. 2018; 1865 (11, Part B): 1745-60.
43. Koran M E I, Hohman T J, Thornton-Wells T A. Genetic interactions found between calcium channel genes modulate amyloid load measured by positron emission tomography. Hum Genet. 2014; 133 (1): 85-93.
44. Zhu L, Li C, Du G, Pan M, Liu G, Pan W, et al. High glucose upregulates myosin light chain kinase to induce microfilament cytoskeleton rearrangement in hippocampal neurons. Molecular medicine reports. 2018; 18 (1): 216-22.
45. Wang R, Reddy P H. Role of Glutamate and NMDA Receptors in Alzheimer's Disease. J Alzheimers Dis. 2017; 57 (4): 1041-8.
46. Braithwaite S P, Stock J B, Lombroso P J, Nairn A C. Protein phosphatases and Alzheimer's disease. Prog Mol Biol Transl Sci. 2012; 106:343-79.
47. Henriques A G, Müller T, Oliveira J M, Cova M, da Cruz e Silva C B, da Cruz e Silva O A B. Altered protein phosphorylation as a resource for potential AD biomarkers. Scientific Reports. 2016; 6 (1): 30319.
48. Lin L, Yee S W, Kim R B, Giacomini K M. SLC transporters as therapeutic targets: emerging opportunities. Nat Rev Drug Discov. 2015; 14 (8): 543-60.
49. Ayka A, Sehirli A O. The Role of the SLC Transporters Protein in the Neurodegenerative Disorders. Clin Psychopharmacol Neurosci. 2020; 18 (2): 174-87.
50. Li Y, Sun H, Chen Z, Xu H, Bu G, Zheng H. Implications of GABAergic Neurotransmission in Alzheimer's Disease. Front Aging Neurosci. 2016; 8:31.
51. Yang C, Qi Y, Sun Z. The Role of Sonic Hedgehog Pathway in the Development of the Central Nervous System and Aging-Related Neurodegenerative Diseases. Front Mol Biosci. 2021; 8:711710-.
52. Vorobyeva A G, Saunders A J. Amyloid-β interrupts canonical Sonic hedgehog signaling by distorting primary cilia structure. Cilia. 2018; 7:5-.
53. Bocharova A, Vagaitseva K, Marusin A, Zhukova N, Zhukova I, Minaycheva L, et al. Association and Gene-Gene Interactions Study of Late-Onset Alzheimer's Disease in the Russian Population. Genes (Basel). 2021; 12 (10): 1647.
54. Liu D, Dai S X, He K, Li G H, Liu J, Liu L G, et al. Identification of hub ubiquitin ligase genes affecting Alzheimer's disease by analyzing transcriptome data from multiple brain regions. 2021; 104 (1): 368504211001146.
55. Deters K D, Nho K, Risacher S L, Kim S, Ramanan V K, Crane P K, et al. Genome-wide association study of language performance in Alzheimer's disease. Brain Lang. 2017; 172:22-9.
56. Lee W S, Lee W-H, Bae Y C, Suk K. Axon Guidance Molecules Guiding Neuroinflammation. Exp Neurobiol. 2019; 28 (3): 311-9.
57. Liu F, Placzek M, Xu H. Axon guidance effect of classical morphogens Shh and BMP7 in the hypothalamicuitary system. Neuroscience letters. 2013; 553:104-9.
58. Jin Y, Cheng X, Lu J, Li X. Exogenous BMP-7 Facilitates the Recovery of Cardiac Function after Acute Myocardial Infarction through Counteracting TGF-beta1 Signaling Pathway. Tohoku J Exp Med. 2018; 244 (1): 1-6.
59. Lowery J W, de Caestecker M P. BMP signaling in vascular development and disease. Cytokine Growth Factor Rev. 2010; 21 (4): 287-98.
60. Licastro F, Chiappelli M, Caldarera C M, Porcellini E, Carbone I, Caruso C, et al. Sharing pathogenetic mechanisms between acute myocardial infarction and Alzheimer's disease as shown by partially overlapping of gene variant profiles. J Alzheimers Dis. 2011; 23 (3): 421-31.
61. Akila Parvathy Dharshini S, Taguchi Yh, Michael Gromiha M. Exploring the selective vulnerability in Alzheimer disease using tissue specific variant analysis. Genomics. 2019; 111 (4): 936-49.
62. Amparan D, Avram D, Thomas C G, Lindahl M G, Yang J, Bajaj G, et al. Direct interaction of myosin regulatory light chain with the NMDA receptor. Journal of neurochemistry. 2005; 92 (2): 349-61.
63. Balabanski L, Serbezov D, Atanasoska M, Karachanak-Yankova S, Hadjidekova S, Nikolova D, et al. Rare genetic variants prioritize molecular pathways for semaphorin interactions in Alzheimer's disease patients. Biotechnology & Biotechnological Equipment. 2021; 35 (1): 1256-62.
64. Dibattista M, Pifferi S, Menini A, Reisert J. Alzheimer's Disease: What Can We Learn From the Peripheral Olfactory System? Front Neurosci. 2020:14:440-.
65. Upadhyay A, Hosseinibarkooie S, Schneider S, Kaczmarek A, Torres-Benito L, Mendoza-Ferreira N, et al. Neurocalcin Delta Knockout Impairs Adult Neurogenesis Whereas Half Reduction Is Not Pathological. Front Mol Neurosci. 2019; 12.
66. Miller J A, Woltjer R L, Goodenbour J M, Horvath S, Geschwind D H. Genes and pathways underlying regional and cell type changes in Alzheimer's disease. Genome medicine. 2013; 5 (5): 48.
67. Upadhyay A, Hosseinibarkooie S, Schneider S, Kaczmarek A, Torres-Benito L, Mendoza-Ferreira N, et al. Neurocalcin Delta Knockout Impairs Adult Neurogenesis Whereas Half Reduction Is Not Pathological. Front Mol Neurosci. 2019; 12:19-.
68. Moss J, Magenheim J, Neiman D, Zemmour H, Loyfer N, Korach A, et al. Comprehensive human cell-type methylation atlas reveals origins of circulating cell-free DNA in health and disease. Nat Commun. 2018; 9 (1): 5068.
69. BAHADO-SINGH RO, VISHWESWARAIAH S, AYDAS B, MISHRA NK, GUDA C, RADHAKRISHNA U. Deep Learning/Artificial Intelligence and Blood-Based DNA Epigenomic Prediction of Cerebral Palsy. International Journal of Molecular Sciences 2019; 20:2075.
70. ALAKWAA F M, CHAUDHARY K, GARMIRE LX. Deep Learning Accurately Predicts Estrogen Receptor Status in Breast Cancer Metabolomics Data. J Proteome Res 2018; 17:337-47.
71. BAHADO-SINGH RO, VISHWESWARAIAH S, ER A, et al. Artificial Intelligence and the detection of pediatric concussion using epigenomic analysis. Brain research 2020; 1726:146510.
72. BAHADO-SINGH RO, VISHWESWARAIAH S, AYDAS B, et al. Artificial intelligence and leukocyte epigenomics: Evaluation and prediction of late-onset Alzheimer's disease. 2021; 16:e0248375.
73. HUANG JH, XIE HL, YAN J, LU HM, XU QS, LIANG YZ. Using random forest to classify T-cell epitopes based on amino acid properties and molecular features. Anal Chim Acta 2013; 804:70-5.
74. MAHADEVAN S, SHAH SL, MARRIE TJ, SLUPSKY CM. Analysis of metabolomic data using support vector machines. Anal Chem 2008; 80:7562-70.
75. LILAND KH. Multivariate methods in metabolomics—from pre-processing to dimension reduction and statistical analysis. TrAC Trends in Analytical Chemistry 2011; 30:827-41.
76. CANDELA, PARMAR V, LEDELL E, ARORA A. Deep Learning with H2O. Number of pages.

What is claimed is:

1. A method of diagnosing or determining the susceptibility to Alzheimer's disease (AD) in a subject in need thereof, wherein the method comprises assaying a biological sample obtained from the subject, comprising cell-free (cf) DNA to determine frequency or percentage of cytosine methylation at one or more loci throughout a genome; and comparing the cytosine methylation level of the sample to the cytosine methylation of a control sample.

2. The method of claim 1, wherein the method further comprises using artificial intelligence (AI) techniques.

3. The method of claim 1 or 2, wherein the method further comprises using (AI) techniques comprising one or more of the following machine learning algorithms: Random Forest (RF), Support Vector Machine (SVM), Linear Discriminant Analysis (LDA), Prediction of Analysis for Microarrays (PAM), Generalized Linear Model (GLM), or deep learning (DL); and optionally wherein the machine learning algorithm is DL.

4. The method of any one of claims 1-3, wherein the method further comprises calculating the subject's risk of developing AD.

5. The method of any one of claims 1-4, wherein the control sample is from one or more normal (healthy) patients or from one or more patients diagnosed with AD.

6. The method of any one of claims 1-5, wherein the biological sample comprises body fluid.

7. The method of any one of claims 1-6, wherein the biological sample comprises blood, plasma, serum, urine, saliva, sputum, sweat, or tears.

8. The method of any one of claims 1-7, wherein the biological sample comprises blood.

9. The method of any one of claims 1-8, wherein the subject is an adult or an elderly adult.

10. The method of any one of claims 1-9, wherein the subject is at least 50 years old, at least 55 years old, at least 60 years old, at least 65 years old, at least 70 years old, or at least 85 years old.

11. The method of any one of claims 1-10, wherein the one or more loci comprise one or more loci from Table 1B, 2B, 3B, or 4B and one of the machine learning algorithms.

12. The method of any one of 1-11, wherein the one or more loci comprise at least two, at least three, at least four, at least five, at least 10, at least 20, at least 30, at least 40, at least 50, at least 60, at least 70, at least 80, or at least 90, or 100 loci from Table 1B, 2B, 3B, or 4B and one of the machine learning algorithms.

13. The method of any one of claims 1-12, wherein the one or more loci comprise an AUC (with 95% CI) of greater than 0.80, 0.85, 0.90, 0.91, 0.92, 0.93, 0.94, 0.95, 0.96, 0.97, 0.98, or 0.99.

14. The method of any one of claims 1-13, wherein the assay is a bisulfite-based methylation assay or a whole-genome methylation assay.

15. The method of any one of claims 1-14, wherein the one or more loci comprise one or more loci or genes from Table 5 or one or more loci from Table 6.

16. The method of any one of claims 1-15, wherein the one or more loci comprise at least two, at least three, at least four, at least five, at least 10, at least 20, at least 30, at least 40, at least 50, at least 60, at least 70, at least 80, or at least 90, or 100 loci from Table 5 or Table 6.

17. The method of any one of claims 1-15, wherein the method further comprises treating the subject.

18. The method of any one of claims 1-16, wherein the method further comprises treating the subject by administering medication.
